(19) United States
(12) Patent Application Publication
(10) Pub. No.: US 2019/0282580 A1
(43) Pub. Date: Sep. 19, 2019

(54) PRO-INFLAMMATORY ROLE FOR ADENOSINE UPTAKE AND METABOLISM

(71) Applicant: La Jolla Institute for Allergy and Immunology, La Jolla, CA (US)

(72) Inventors: Sonia SHARMA, San Diego, CA (US); Rekha DHANWANI, San Diego, CA (US); Mariko TAKAHASHI, San Diego, CA (US)

(21) Appl. No.: 16/349,184

(22) PCT Filed: Nov. 10, 2017
(86) PCT No.: PCT/US2017/061160

§ 371 Date: May 10, 2019

Related U.S. Application Data
(60) Provisional application No. 62/420,443, filed on Nov. 10, 2016.
(60) Provisional application No. 62/563,578, filed on Sep. 26, 2017.

(51) Int. Cl. A61K 31/519

(52) U.S. Cl. CPC A61K 31/519

(57) ABSTRACT

Disclosed herein, in certain embodiments, are methods of modulating an immune response via the modulation of the adenosine reuptake, adenosine salvage, or adenosine degradation pathways of a cell. In certain embodiments, the invention relates to adenosine reuptake inhibitors, adenosine salvage pathway inhibitor or adenosine degradation pathway inhibitors, adenosine, deoxyadenosine, and variants or derivatives thereof, and uses thereof, for example the treatment of autoimmune conditions, pro-inflammatory conditions, neoplasia, neoplastic disorder, or cancer. In certain embodiments, the invention relates to a compound or composition that modulates the adenosine uptake, adenosine salvage, or adenosine degradation pathway and uses thereof.

### RELATED APPLICATIONS

This application claims priority to U.S. Provisional Patent Application No. 62/563,578, filed Sep. 26, 2017; and U.S. Provisional Patent Application No. 62/420,443, filed Nov. 10, 2016. The entire contents of the foregoing applications are incorporated herein by reference, including all text, tables, sequence listing and drawings.

### GOVERNMENT SUPPORT

This invention was made with government support under grant number R01 CA199376-03 awarded by the National Cancer Institute, a division of The National Institutes of Health. The Government has certain rights in the invention.

### FIELD OF THE INVENTION

Embodiments of the invention relate to modulating an immune response via the modulation of the adenosine reuptake, adenosine salvage, or adenosine degradation pathways of a cell. In certain embodiments, the invention relates to adenosine reuptake inhibitors, adenosine salvage pathway inhibitors or adenosine degradation pathway inhibitors, adenosine, deoxyadenosine, and variants or derivatives thereof, and uses thereof, for example the treatment of autoimmune conditions, pro-inflammatory conditions, neoplasia, neoplastic disorder, or cancer. In certain embodiments, the invention relates to a compound or composition that modulates the adenosine uptake, adenosine salvage, or adenosine degradation pathway and uses thereof.

### INTRODUCTION

Vasculitis syndromes are often auto-immune diseases characterized by inflammation of blood vessels, which promotes vessel weakening, thickening and scarring, ultimately restricting blood flow and precipitating organ and tissue damage. Polyarteritis nodosa (PAN) vasculitis is a systemic, necrotizing vasculitis affecting medium-sized vessels of skin, kidney, heart, gastrointestinal tract, joints and nerves. Left untreated, most PAN cases are fatal due to kidney failure, myocardial infarction or stroke.

### SUMMARY

Disclosed herein is a method for modulating an immune response, the method comprising inhibiting adenosine reuptake, adenosine salvage or adenosine degradation pathways of a cell. In some embodiments, a cell is an endothelial cell. In still other embodiments, an immune response is an innate immune response.

In some embodiments, a method comprises modulating Adenosine Deaminase 2 expression or activity or Adenosine Deaminase 1 expression or activity. In certain other embodiments, a method comprises inhibiting Adenosine Deaminase 1 expression or activity. In certain alternative embodiments, a method comprises contacting the cell with an agent that inhibits Adenosine Deaminase 1 expression or activity.

In certain embodiments, a method comprises stimulating expression or activity of one or more of AHCY, DNMT1, DNMT3a and DNMT3b. In still other embodiments, a method comprises contacting the cell with an agent that stimulates expression or activity of one or more of AHCY, DNMT1, DNMT3a and DNMT3b.

In some embodiments, a method comprises contacting the cell with an adenosine reuptake inhibitor. In certain embodiments, an adenosine reuptake inhibitor is an acadesine, acetate, barbiturates, benzodiazepines, calcium channel blockers, carbamazepine, carisoprodol, cilostazol, cyclobenzaprine, dilazep, dipyridamole, estradiol, ethanol, flumazenil, hexobendine, hydroxyzine, indomethacin, inosine, KF24345, meprobamate, nitrobenzylthioguanosine, nitrobenzylthioinosine, papaverine, pentoxifylline, phenothiazines, phenytoin, progesterone, propentofylline, propofol, puromycin, r75231, soluflazine, toyocamycin, tracazolate, or tricyclic antidepressant. In still other embodiments, an adenosine reuptake inhibitor is dipyridamole.

In certain embodiments, a method comprises inhibiting, blocking or decreasing an immune response. In other embodiments, a method comprises modulating an immune response in a subject having an autoimmune disorder or proinflammatory condition. In certain embodiments, an autoimmune disorder or pro-inflammatory condition is selected from the group consisting of polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-esophageal reflux disease, erythroderma, Behcet's disease, amyotrophic lateral sclerosis, transplantation, rheumatoid arthritis, juvenile rheumatoid arthritis, malignant rheumatoid arthritis, Drug-Resistant Rheumatoid Arthritis, Neuromyelitis optica, Kawasaki disease, polyarticular or systemic juvenile idiopathic arthritis, psoriasis, chronic obstructive pulmonary disease (COPD), Castleman's disease, asthma, allergic asthma, allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenic purpura, pernicious anemia, sensorineural hearing loss, idiopathic bilateral progressive sensorineural hearing loss, autoimmune polyglandular syndrome type I or type II, immune infertility and immune-mediated infertility.

In some embodiments, an autoimmune disorder or pro-inflammatory condition is a vasculitis syndrome. In certain other embodiments, a vasculitis syndrome is polyarteritis nodosa (PAN). In still other embodiments, a vasculitis syndrome comprises an adenosine deaminase 2 deficiency (ADA2). In certain embodiments, a vasculitis syndrome comprises Aicardi-Goutières Syndrome (AGS), TREX1 (AGS1 vasculitis), or STING (SAVI vasculitis).

In certain embodiments, an autoimmune disorder or pro-inflammatory condition is a symptom or side-effect of cancer. In alternative embodiments, a pro-inflammatory condition comprises an adenosine deaminase 2 deficiency.

In some embodiments, the subject has, or is suspected of having a viral, bacterial or fungal infection. In certain embodiments, a viral infection comprises an infection of Hepatitis B virus (HBV), Hepatitis C virus (HCV), Epstein-Barr virus (EBV) or cytomegalovirus (hCMV).

In certain embodiments, an autoimmune disorder or pro-inflammatory condition is not asthma.

In some embodiments, an inhibitor is a protein, peptide, small molecules, antibody, bispecific antibody, antibody derivative, ligand mimetic, nucleic acid or pharmaceutical composition.

Disclosed herein is a method of treating a subject having or suspected of having an autoimmune disorder, or pro-inflammatory condition comprising: a) providing a subject having, or suspected of having, an autoimmune disorder, or pro-inflammatory condition; and b) administering a therapeutically effective amount of an adenosine reuptake inhibitor, adenosine salvage pathway inhibitor or adenosine degradation pathway inhibitor to the subject. In certain embodiments, an adenosine reuptake inhibitor is selected from the group consisting of acadesine, acetate, barbiturates, benzodiazepines, calcium channel blockers, carbamazepine, carisoprodol, cilostazol, cyclobenzaprine, dilazep, dipyridamole, estradiol, ethanol, flumazenil, hexobendine, hydroxyzine, indomethacin, inosine, KF24345, meprobamate, nitrobenzylthioguanosine, nitrobenzylthioinosine, papaverine, pentoxifylline, phenothiazines, phenytoin, progesterone, propentofylline, propofol, puromycin, r75231, soluflazine, toyocamycin, tracazolate, and tricyclic antidepressants. In still other embodiments, the adenosine reuptake inhibitor is dipyridamole.

In certain embodiments, a method comprises administering an inhibitor of Adenosine Deaminase 1. In certain alternative embodiments, a method comprises administering a stimulator of AHCY, DNMT1, DNMT3a and DNMT3b. In some embodiments, an adenosine reuptake inhibitor, adenosine, deoxyadenosine, or variant or derivative thereof, is administered by intravenous (i.v.) administration to the subject. In still other embodiments, the adenosine reuptake inhibitor, the adenosine, the deoxyadenosine, or the variant or derivative thereof, is administered at a dose of 0.01 mg to 50 mg, 0.01 mg to 12 mg, 0.01 to 6 mg, 0.01 mg to 5 mg or 0.01 mg to 1 mg.

In some embodiments, an autoimmune disorder or pro-inflammatory condition is selected from the group consisting of polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-esophageal reflux disease, erythroderma, Behcet's disease, amyotrophic lateral sclerosis, transplantation, Neuromyelitis Optica, rheumatoid arthritis, juvenile rheumatoid arthritis, malignant rheumatoid arthritis, Drug-Resistant Rheumatoid Arthritis, Kawasaki disease, polyarticular or systemic juvenile idiopathic arthritis, psoriasis, chronic obstructive pulmonary disease (COPD), Castleman's disease, asthma, allergic asthma, allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenic purpura, pernicious anemia, sensorineural hearing loss, idiopathic bilateral progressive sensorineural hearing loss, autoimmune polyglandular syndrome type I or type II, immune infertility and immune-mediated infertility.

In certain embodiments, an autoimmune disorder or pro-inflammatory condition is a vasculitis syndrome. In other embodiments, a vasculitis syndrome is polyarteritis nodosa (PAN). In certain specific embodiments, a vasculitis syndrome comprises an adenosine deaminase 2 deficiency (ADA2). In alternative embodiments, a vasculitis syndrome comprises Aicardi-Goutières Syndrome (AGS), TREX1 (AGS1 vasculitis), or STING (SAVI vasculitis). In some embodiments, a pro-inflammatory condition comprises an adenosine deaminase 2 deficiency.

In some embodiments, an autoimmune disorder or pro-inflammatory condition is a symptom or side-effect of cancer. In still other embodiments, a subject has, or is suspected of having a viral, bacterial or fungal infection. In certain embodiments, a viral infection comprises an infection of Hepatitis B virus (HBV), Hepatitis C virus (HCV), Epstein-Barr virus (EBV) or cytomegalovirus (hCMV). In alternative embodiments, an autoimmune disorder or pro-inflammatory condition is not asthma.

Disclosed herein is a method of modulating an immune response, the method comprising stimulating the adenosine reuptake, adenosine salvage or adenosine degradation pathways of a cell. In certain embodiments, a cell is an endothelial cell. In alternative embodiments, an immune response is an innate immune response.

In some embodiments, a method comprises modulating Adenosine Deaminase 2 expression or activity or Adenosine Deaminase 1 expression or activity. In still other embodiments, a method comprises stimulating Adenosine Deaminase 1 expression or activity. In certain specific embodiments, a method comprises contacting the cell with an agent that stimulates Adenosine Deaminase 1 expression or activity.

In certain embodiments, a method comprises inhibiting expression or activity of one or more of AHCY, DNMT1, DNMT3a and DNMT3b. In other embodiments, a method comprises contacting the cell with an agent that inhibits expression or activity of one or more of AHCY, DNMT1, DNMT3a and DNMT3b. In some embodiments, a method comprises contacting the cell with an adenosine reuptake stimulator.

In some embodiments, a method comprises increasing, stimulating, eliciting or promoting an immune response. In certain alternative embodiments, a method comprises modulating an immune response in a subject having a neoplasia, neoplastic disorder or cancer. In still other embodiments, a neoplasia, neoplastic disorder or cancer comprises a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma. In other embodiments, a neoplasia, neoplastic disorder or cancer comprises hematopoietic cells. In alternative embodiments, a sarcoma comprises a lymphosarcoma, liposarcoma, osteosarcoma, chondrosarcoma, leiomyosarcoma, rhabdomyosarcoma or fibrosarcoma. In still other embodiments, a neoplasia, neoplastic disorder or cancer comprises a myeloma, lymphoma or leukemia. In certain embodiments, a neoplasia, neoplastic disorder or cancer comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer.

In certain embodiments, a lung neoplasia, neoplastic disorder or cancer comprises small cell lung or non-small cell lung cancer. In alternative embodiments, a neoplasia, neoplastic disorder or cancer comprises a stem cell neoplasia, neoplastic disorder or cancer. In alternative embodiments, a method inhibits, or reduces relapse or progression of the neoplasia, neoplastic disorder or cancer.

Disclosed herein is a method of treating a subject having a neoplasia, neoplastic disorder or cancer comprising: a) providing a subject having, or suspected of having, a neoplastic disorder; and b) administering a therapeutically effective amount of a compound or composition that stimulates adenosine uptake or the adenosine salvage or degradation pathways.

Also disclosed herein is a method of reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy, comprising administering to the subject an amount of a compound or composition that stimulates adenosine uptake or the adenosine salvage or degradation pathway sufficient to reduce or inhibit metastasis of the neoplasia, tumor, cancer or malignancy to other sites, or formation or establishment of metastatic neoplasia, tumor, cancer or malignancy at other sites distal from the primary neoplasia, tumor, cancer or malignancy.

In certain embodiments, a neoplasia, neoplastic disorder, tumor, cancer or malignancy comprises a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma. In certain other embodiments, a neoplasia, neoplastic disorder, tumor, cancer or malignancy comprises hematopoietic cells. In some embodiments, sarcoma comprises a lymphosarcoma, liposarcoma, osteosarcoma, chondrosarcoma, leiomyosarcoma, rhabdomyosarcoma or fibrosarcoma. In still other embodiments, a neoplasia, neoplastic disorder, tumor, cancer or malignancy comprises a myeloma, lymphoma or leukemia.

In some embodiments, a neoplasia, neoplastic disorder, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer. In certain embodiments, a lung neoplasia, neoplastic disorder, tumor, cancer or malignancy comprises small cell lung or non-small cell lung cancer. In alternative embodiments, a neoplasia, neoplastic disorder, tumor, cancer or malignancy comprises a stem cell neoplasia, tumor, cancer or malignancy. In certain embodiments, a method inhibits, or reduces relapse or progression of the neoplasia, neoplastic disorder, tumor, cancer or malignancy.

In certain embodiments, the method further comprising administering an anti-cell proliferative, anti-neoplastic, anti-tumor, anti-cancer or immune-enhancing treatment or therapy. In some embodiments, a treatment results in partial or complete destruction of the neoplastic, tumor, cancer or malignant cell mass; a reduction in volume, size or numbers of cells of the neoplastic, tumor, cancer or malignant cell mass; stimulating, inducing or increasing neoplastic, tumor, cancer or malignant cell necrosis, lysis or apoptosis; reducing neoplasia, tumor, cancer or malignancy cell mass; inhibiting or preventing progression or an increase in neoplasia, tumor, cancer or malignancy volume, mass, size or cell numbers; or prolonging lifespan. In still other embodiments, a treatment results in reducing or decreasing severity, duration or frequency of an adverse symptom or complication associated with or caused by the neoplasia, tumor, cancer or malignancy. In still other embodiments, a treatment results in reducing or decreasing pain, discomfort, nausea, weakness or lethargy. In certain other embodiments, a treatment results in increased energy, appetite, improved mobility or psychological well-being.

In some embodiments, a method comprises modulating Adenosine Deaminase 2 expression or activity or Adenosine Deaminase 1 expression or activity. In certain other embodiments, a method comprises stimulating Adenosine Deaminase 1 expression or activity. In certain embodiments, a method comprises contacting the cell with an agent that stimulates Adenosine Deaminase 1 expression or activity. In still other embodiments, a method comprises inhibiting expression or activity of one or more of AHCY, DNMT1, DNMT3a and DNMT3b. In particular embodiments, a method comprises contacting the cell with an agent that inhibits expression or activity of one or more of AHCY, DNMT1, DNMT3a and DNMT3b. In certain embodiments, wherein the method comprises contacting the cell with an adenosine reuptake stimulator.

Disclosed herein is a method of treating a subject having or suspected of having an autoimmune disorder, or pro-inflammatory condition comprising: a) providing a subject having, or suspected of having, an autoimmune disorder, or pro-inflammatory condition; and b) stimulating expression or activity of one or more of AHCY, DNMT1, DNMT3a and DNMT3b in the subject. In some embodiments, a method comprises administering to the subject a therapeutically effective amount of an agent that stimulates expression or activity of one or more of AHCY, DNMT1, DNMT3a and DNMT3b.

Disclosed herein is a method of treating a subject having a neoplasia, neoplastic disorder or cancer comprising: a) providing a subject having, or suspected of having, a neoplastic disorder; and b) administering a therapeutically effective amount of an inhibitor of expression or activity of one or more of AHCY, DNMT1, DNMT3a and DNMT3b. In certain embodiments, a method comprises administering to the subject a therapeutically effective amount of an agent that inhibits expression or activity of one or more of AHCY, DNMT1, DNMT3a and DNMT3b.

Disclosed herein is a method of treating a subject having or suspected of having an autoimmune disorder, or pro-inflammatory condition comprising: a) providing a subject having, or suspected of having, an autoimmune disorder, or pro-inflammatory condition; and b) administering a therapeutically effective amount of an analogue of adenosine, deoxyadenosine, a variant or derivative thereof, to the subject. In some embodiments, the analogue inhibits the activity of adenosine, or deoxyadenosine.

Disclosed herein is a method of treating a subject having a neoplasia, neoplastic disorder or cancer comprising: a) providing a subject having, or suspected of having a neoplasia, neoplastic disorder or cancer; and b) administering a therapeutically effective amount of adenosine, deoxyadenosine, or a variant or derivative thereof, or an analogue of adenosine or deoxyadenosine to the subject. In some embodiments, an analogue of adenosine or deoxy adenosine mimics or agonizes the biological effects of adenosine, or deoxyadenosine.

In some aspects, disclosed herein is a method of treating a subject having or suspected of having an autoimmune disorder, or pro-inflammatory condition. In some embodiments, the method comprises providing a subject having, or suspected of having, an autoimmune disorder, or pro-inflammatory condition, and administering a therapeutically effective amount of adenosine reuptake inhibitor to the subject.

In some aspects, disclosed herein is a method of treating a subject having or suspected of having an autoimmune disorder, or pro-inflammatory condition comprising providing a subject having, or suspected of having, an autoimmune disorder, or pro-inflammatory condition and administering a therapeutically effective amount of adenosine, deoxyadenosine, a variant or derivative thereof, to the subject.

In some aspects, disclosed herein is a method of treating a subject having a neoplasia, neoplastic disorder or cancer comprising providing a subject having, or suspected of having, a neoplastic disorder, and administering a therapeutically effective amount of a compound or composition that stimulates adenosine uptake or the adenosine salvage pathway.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows cell-intrinsic innate immune IFN-I response through the STING pathway.

FIG. 2 shows that the cGAS-STING-TBK1 axis regulates DNA-induced IRF3 and IFNβ responses to DNA. HUVEC:umbilical vein endothelial cells; HAEC: aorticendothelial cells; MO: macrophages; mo: monocytes; BEC: bronchial epithelial cells.

FIG. 3 shows that kidney, brain, skin and murine aortic endothelial cells mount robust IRF3 (a) and IFNβ (b) responses to DNA. Cells were verified for expression of endothelial markers (CD31, CD34 and ICAM1) by flow cytometry.

FIG. 4 shows that depletion of ADA2 using siRNA (a, b), shRNA (c) or CRISPR/Cas9 (d) in primary endothelial cells enhances IRF3 activation (a, c, d) and pro-inflammatory cytokine production (b) after DNA stimulation (a) or hCMV infection.

FIG. 5 shows that kidney, brain, skin and HUVEC endothelial cells express robust levels of ADA2 mRNA. FIG. 5a shows ADA2 mRNA measured by TaqMan qRT-PCR in primary human endothelial cells and U937 monocytes. FIG. 5b shows percent knock down of ADA2 mRNA by individual siRNA. FIG. 5c shows ADA2 protein measured by ELISA.

FIG. 6 shows that ADA1 depletion by siRNAs in HUVEC (a) does not enhance IFNβ induction by hCMV (b). ADA2 depletion was used as a positive control (b).

FIG. 7 shows that supplementation with extracellular Ado enhances IRF3 activation (a) and IFNβ induction (b) in primary human endothelial cells.

FIG. 8 shows quantification of 2'S′-cGAMP in HUVEC using LC-MS/MS-based multiple reaction monitoring of the 344.0 Da ionization fragment of cGAMP.

FIG. 9 shows two distinct consequences of extracellular Adenosine (Ado). (1) Signaling through purinergenic Adenosine Receptors (right). (2) Uptake into cells via ENT transporters and intracellular catabolism (left).

FIG. 10 shows mRNA levels of ENT1-4 analyzed by TaqMan qRT-PCR in primary human endothelial cells.

FIG. 11 shows three metabolic consequences of Ado/dAdo that can be analyzed by monitoring levels of each metabolite in control and ADA2-deficient endothelial cells, either left unstimulated or stimulated with DNA/hCMV.

FIG. 12 shows dipyridamole causes dose-dependent decrease in hCMV induced IFNβ in siControl and siADA2-treated endothelial cells isolated from umbilical cord (HUVEC), brain (HBMVEC) and skin (HDMVEC).

FIGS. 13A and 13B shows RNAi depletion of ADA2/CECR1 enhances IRF3 translocation and pro-inflammatory cytokine production in HUVEC after viral infection.

FIGS. 14A and 14B show that supplementation with extracellular adenosine (Ado) and deoxyadenosine (dAdo) enhances IRF3 translocation and pro-inflammatory cytokine production after viral infection.

FIGS. 15A, 15B and 15C show that NECA, a non-hydrolyzable analogue of adenosine that signals through adenosine receptors, has no effect on IRF3.

FIG. 16 shows that the adenosine reuptake inhibitor dipyramidole reduces cytokine production in ADA2/CECR1-depleted cells.

FIG. 17 shows that ADA2 is a negative regulator of cellular the innate immune response. FIG. 17a shows a non-limiting example of inducible nuclear translocation of IRF3 and IFNβ production in primary human endothelial cells (HUVEC, HAEC), macrophages (MO), monocytes (mo) and bronchial epithelial cells (NHBE) upon dsDNA treatment. FIG. 17b shows a non-limiting example of nuclear IRF3 translocation and IFNβ production in kidney, brain and skin-derived endothelial cells upon dsDNA treatment. FIG. 17c shows a non-limiting example of nuclear IRF3 translocation in siControl, siADA2 and siTREX1-treated HUVEC upon dsDNA treatment or hCMV infection. FIG. 17d shows a non-limiting example of nuclear IRF3 translocation in HUVEC treated with multiple ADA2-specific siRNAs. FIG. 17e shows a non-limiting example of induction of IFNβ mRNA and protein in siControl, siADA2 and siTREX1-treated HUVEC. FIG. 17f shows a non-limiting example of induction of pro-inflammatory and anti-viral ISG in siControl, siADA2 and siTREX1-treated HUVEC. FIG. 17g shows a non-limiting example of volcano plots representing whole-transcriptome RNAseq analysis in siControl, siADA2 and siTREX1-treated HUVEC upon infection with HCMV. FIG. 17h shows a non-limiting example of a heatmap representation of gene subsets up-regulated in ADA2-depleted cells upon infection with HCMV. Data shown are representative of results from at least three independent experiments. Values are shown as mean +/− standard deviation. ND, not detectable. *, P≤0.05; **, P≤0.01; ***P≤0.001; NS, not significant.

FIG. 18 shows that cellular uptake and metabolism of purine nucleosides drives innate immune gene expression. FIG. 18a shows a non-limiting example of ADA2 mRNA and protein expression in primary human endothelial cells and U937 monocytes. FIG. 18b shows a non-limiting example of immunohistochemical staining of ADA2 in U937 monocytes and ADA2 and CD31 in normal human skin tissue. FIG. 18c shows a non-limiting example of IRF3 translocation and IFNβ mRNA induction in cells supplemented with adenosine (Ado), deoxyadenosine (d-Ado) and deoxyadenosine and pentostatin (d-Ado/pento). FIG. 18d shows a non-limiting example of a schematic representation of adenosine receptor signaling and cellular uptake and metabolism. FIG. 18e shows via a non-limiting example that supplementation with 5′-N-ethylcarboxamidoadenosine (NECA) does not significantly affect nuclear IRF3 translocation or IFNβ mRNA induction. FIG. 18f shows a non-limiting example of IFNβ mRNA induction in siControl and siADA2-treated cells after pre-treatment with the adenosine reuptake inhibitor dipyridamole (DPM) upon infection with HCMV. FIG. 18g shows a non-limiting example of a heatmap representation of gene subsets up-regulated in d-Ado/pento and DPM-treated cells, as determined by whole-transcriptome RNAseq analysis. Data shown are representative of results from at least three independent experiments. Values are shown as mean +/− standard deviation. *, P≤0.05; **, P≤0.01; ***P≤0.001; NS, not significant.

FIG. 19 shows Dyregulated SAM pathway metabolism in ADA2-deficient cells FIG. 19a shows a non-limiting example of metabolite profiling in siControl and siADA2-treated HUVEC using untargeted LC-MS/MS. FIG. 19b shows a non-limiting example of intracellular purine metabolism via the salvage pathway (ADA1) and degradation pathway (ADK, DCK). FIG. 19c shows a non-limiting example of fold change in purine salvage and degradation metabolites, as measured in siControl and siADA2-treated HUVEC lysates and supernatants using untargeted LC-MS/MS. FIG. 19d shows a non-limiting example of functional mapping of metabolic enzymes in the purine salvage and degradation pathways using IFNβ mRNA induction upon dsDNA treatment. FIG. 19e shows a non-limiting example of IFNβ mRNA induction HUVEC supplemented with d-no upon dsDNA treatment. FIG. 19f shows a non-limiting example of IFNβ mRNA induction in siControl, siSAMHD1 and siAHCY-treated cells upon dsDNA treatment. FIG. 19g shows a non-limiting example of fold change of SAM pathway metabolites, as measured in siControl and siADA2-treated HUVEC lysates using untargeted LC-MS/MS. FIG. 19h shows a non-limiting example of genomic DNA methylation. Data shown are representative of results from at least three independent experiments. Values are shown as mean +/− standard deviation. *, P≤0.05; **, P≤0.01; ***P≤0.001; NS, not significant.

FIG. 20 shows via a non-limiting example that DNA hypomethylation drives over-expression of STING, IFNβ and other innate genes/FIG. 20a (left) provides via Venn diagram a non-limiting example demonstrating overlap between hCMV-induced genes altered in siControl, siADA2 and siTREX1-treated cells; while FIG. 20a (right) shows a non-limiting example of RNAseq reads mapping to the STING gene in siControl, siADA2 and siTREX1-treated cells. FIGS. 20b and 20c show expression of STING mRNA, measured by TaqMan qRT-PCR, in control, siADA2, pento-/dAdo and dIno-treated cells. 20c shows expression of STING and cGAS protein, detected by western blotting, in siControl, siADA2 and siTREX1-transfected cells upon dsDNA treatment (200 ng/mL) for 3 h. 20d shows IRF3 phosphorylation at S396, detected by western blotting, in siControl, siADA2 and siTREX1-transfected HUVEC upon dsDNA treatment (200 ng/mL) for 3 h. 20e shows TBK1 auto-phosphorylation at 5172, detected by western blotting, in siControl, siADA2 and siTREX1-transfected cells upon dsDNA treatment (200 ng/mL) for 3 h. 20f shows measurements of endogenous 2′5′-cGAMP levels in siControl, siADA2, siTREX1 and sicGAS-treated HUVEC lysates using targeted LC-MS/MS. Data are representative of results from at least three independent experiments. WGBS data are representative of one experiment using three biological replicates for each sample. Values are presented as mean +/− standard deviation. ND, not detectable. *, P≤0.05; **, P≤0.01; ***P≤0.001.

FIG. 21a shows a non-limiting example of the degree of IRF3 co-localization with nuclear DAPI being uniformly higher at the single-cell level. FIG. 20b shows a non-limiting example of IRF3 and IFNβ responses to dsDNA being abolished by depletion of STING. FIGS. 21c-21f shows a non-limiting example demonstrating that depletion of ADA2 significantly enhanced nuclear IRF3 accumulation upon transfection of immuno-stimulatory dsDNAs or infection with human cytomegalovirus (hCMV). FIGS. 21g-21i shows a non-limiting example of whole-transcriptome RNAseq confirming higher induction of ISG in ADA2-depleted cells, and showing that more genes were significantly altered.

FIGS. 22a and 22b show non-limiting examples of how disruption of ADA2 and dAdo supplementation enhanced IFNβ induction. FIG. 22c shows a non-limiting example of dAdo/Pento-supplemented HUVEC confirming profound changes in many immuno-modulatory genes and ISG. FIG. 22d shows a non-limiting example of AdoR signaling, wherein cells were supplemented with 5′-N-ethylcarboxamidoadenosine (NECA), a non-hydrolyzable analogue of Ado, to stimulate AdoR expressed by HUVEC. FIGS. 22e-g show non-limiting examples of rapid uptake of circulating Ado/d-Ado by the vascular endothelium occurring within a few seconds²²⁻²⁴, consistent with the relatively high levels of Equilibrative Nucleoside Transporters (ENT) expressed by these cells.

FIGS. 23a-c show non-limiting example of augmented IFNβ induction.

FIG. 24 shows, via a non-limiting example, that production of the STING agonist cGAMP, which is produced upon activation of the dsDNA sensor/enzyme cGAS, was unchanged in siADA2-treated cells. FIG. 24a shows the chemical structure of cGAMP. FIG. 24b shows LC-MS/MS peaks for cGAMP. FIG. 24c shows linear relation between peak area and cGAMP concentration. FIG. 24d shows measurements of endogenous 2′5′-cGAMP levels in siControl, siADA2, siTREX1 and sicGAS-treated HUVEC lysates using targeted LC-MS/MS.

FIG. 25a shows that ADA2 is highly expressed in immune cells. FIG. 25b shows 39 human disease-linked genes screened in an arrayed format using gene-specific siRNAs to transfect HUVEC 72 h prior to dsDNA stimulation and analysis of IRF3 nuclear translocation. FIG. 25c shows IFNβ mRNA induction, measured by TaqMan qRT-PCR, in siControl, siDNMT1, siDNMT3a, and siDNT3b-transfected HUVEC upon dsDNA treatment (200 ng/mL) for 3 h.

FIG. 26a shows siRNA knockdown of additional metabolic enzymes in the purine salvage and degradation pathways in ADA2-depleted cells followed by analysis of IFNβ induction, measured by TaqMan qRT-PCR, upon dsDNA treatment (200 ng/mL) for 3 h. FIG. 26b shows the fold change in the downstream SAM cycle metabolite L-cytstathionine, measured in siControl and siADA2-treated HUVEC lysates or Vehicle and dAdo/Pento-supplemented supernatants using untargeted LC-MS/MS. FIG. 26c shows a flux analysis of SAM cycle using ¹³C-labelled methionine followed by quantification of intracellular pools of labeled methionine, SAM, and SAH, measured in siControl and siADA2-treated HUVEC lysates using LC-MS/MS. FIG. 26d shows IFNβ mRNA induction, measured by TaqMan qRT-PCR, in siControl, siDNMT1, siDNMT3a, and siDNT3b-transfected HUVEC upon dsDNA treatment (200 ng/mL) for 3 h. FIG. 26f shows data representative of results from at least three independent experiments. WGBS data are representative of one experiment using three biological replicates for each sample. Values are presented as mean +/− standard deviation. *, P≤0.05; **, P≤0.01; ***P≤0.001.

### DETAILED DESCRIPTION

The current inventors have developed methods for modulating an immune response by modulating the adenosine reuptake, adenosine salvage or adenosine degradation pathways of a cell. In certain embodiments, the present invention comprises modulating the adenosine reuptake, adenosine salvage or adenosine degradation pathways in an endothelial cell.

As used herein an immune response may refer to an innate immune response, an adaptive immune response or a recall immune response and in certain embodiments, may include expression, stimulation, activation, proliferation, number or activity of B cells, T cells, macrophages, monocytes, dendritic cells, cytokines or other enzyme or proteins involved in promoting or inhibiting an immune response.

In some embodiments, disclosed herein is a method of treating a subject having or suspected of having, an autoimmune conditions and pro-inflammatory conditions. In certain embodiments, a method of treating a subject comprises administering a therapeutically effective amount of an adenosine reuptake inhibitor to a subject. In certain embodiments, a method of treating a subject comprises administering adenosine, deoxyadenosine or a variant or derivative thereof to a subject. In certain embodiments, a pro-inflammatory condition comprises an adenosine deaminase 2 deficiency or is induced by an infectious agent (e.g., a virus, bacteria or parasite).

The genetic basis for PAN vasculitis was identified as a deficiency of Adenosine Deaminase 2 (ADA2), the enzyme that metabolizes circulating adenosine, a potently immune-modulatory nucleoside. Also, there is a clear link between PAN vasculitis and virus infection. Accordingly, data herein shows that depleting ADA2 in primary human vascular endothelial cells using RNA interference (RNAi) provides significant enhancement of innate immune responses to virus infection, specifically through enhanced secretion of pro-inflammatory cytokines. Therefore, ADA2 functions to dampen anti-viral innate immune responses in endothelial cells. Upon viral infection, ADA2 deficiency results in over-production of cytokines that drive endothelial activation, immune cell recruitment, and vessel inflammation.

In addition to ADA2, the inventors have discovered that modulation of other molecules including ADA1, AHCY, DNMT1, DNMT3a and DNMT3b can modulate immune response, including for treatment of disease.

Vasculitis refers to any auto-immune disease that causes inflammation and damage to blood vessels, which can precipitate multi-organ damage due to restricted blood flow to the affected tissues. Left untreated, severe vasculitis is a debilitating and often fatal disease. Many vasculitis diseases are rare genetic syndromes, and unraveling the biological basis of these diseases is absolutely critical for developing effective therapeutic treatments for patients. Polyarteritis Nodosa (PAN) vasculitis is caused by immune responses originating directly from the vascular endothelium itself. Accordingly, vascular endothelial cells represent an important source of inflammation in vasculitis. Novel treatments are disclosed herein to specifically dampen pro-immune and pro-inflammatory responses of the vasculature.

Polyarteritis nodosa (PAN) vasculitis is a systemic, necrotizing vasculitis affecting medium-sized arteries in skin, kidney, heart, gastrointestinal tract, joints and nerves. Patients suffering from this multi-system auto-immune disease experience recurrent fevers, sweats, weight loss, fatigue, pain in the abdomen, muscles or joints and seizures. Left untreated, most patients succumb to kidney failure, myocardial infarction or stroke. Whole-exome sequencing revealed the genetic basis of PAN to be a loss-of-function mutations in the gene encoding Adenosine Deaminase 2 (ADA2). In immune cells of PAN patients, deficiency of ADA2 strongly skews immature monocyte differentiation towards a pro-inflammatory M1 phenotype, providing some mechanistic basis for auto-inflammation.

There is a clear but poorly understood link between PAN symptoms and infection with viral pathogens. As shown herein RNAi depletion of ADA2 in endothelial cell results in significantly enhanced production of innate immune, pro-inflammatory cytokines induced by virus infection. Based upon these exciting new results, it was determined herein that ADA2 functions by dampening anti-viral innate immune responses in endothelium. Thus, in virally-infected endothelial cells, deficiency of ADA2 results in over-production of pro-inflammatory cytokines that precipitate inflammation via endothelial activation and leukocyte recruitment.

Infection is a well-recognized trigger of vasculitis, with many viral, bacterial and fungal pathogens associated with symptoms of vascular inflammation. PAN vasculitis is linked to infection with Hepatitis B virus (HBV), Hepatitis C virus (HCV), Epstein-Barr virus (EBV), Cytomegalovirus (CMV) and Parvovirus. In fact, >30% of PAN cases occur secondary to HBV infection, which used to be the major criteria for confirming diagnosis. Interestingly, many cases of HBV-PAN involve direct infection of endothelial cells at vascular lesions, implicating viral replication in blood vessels as a direct cause of injury or inflammation. Accordingly, HBV-PAN is responsive to anti-viral therapies that limit virus replication in cells. While most cases of PAN vasculitis are idiopathic, it is notable that EBV, CMV and Parvovirus—which can all infect vascular endothelial cells—are often asymptomatic, raising the possibility that these viral infections play a common role in eliciting PAN vasculitis.

Disclosed herein are methods and uses of treating a subject having or suspected of having an autoimmune disorder, or pro-inflammatory condition. Non-limiting examples of an autoimmune disorder or pro-inflammatory condition include polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-esophageal reflux disease, erythroderma, Behcet's disease, amyotrophic lateral sclerosis, transplantation, Neuromyelitis Optica, rheumatoid arthritis, juvenile rheumatoid arthritis, malignant rheumatoid arthritis, Drug-Resistant Rheumatoid Arthritis, Neuromyelitis optica, Kawasaki disease, polyarticular or systemic juvenile idiopathic arthritis, psoriasis, chronic obstructive pulmonary disease (COPD), Castleman's disease, asthma, allergic asthma, allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenic purpura, pernicious anemia, sensorineural hearing loss, idiopathic bilateral progressive sensorineural hearing loss, autoimmune polyglandular syndrome type I or type II, immune infertility and immune-mediated infertility.

In certain embodiments, an autoimmune disorder or pro-inflammatory condition is a vasculitis syndrome. Non-limiting examples of a vasculitis syndrome include polyarteritis nodosa (PAN), an adenosine deaminase 2 deficiency (ADA2), Aicardi-Goutières Syndrome (AGS), TREX1 (AGS1 vasculitis), and STING (SAVI vasculitis). In some embodiments, a pro-inflammatory condition comprises an adenosine deaminase 2 deficiency.

Some pro-inflammatory conditions or autoimmune conditions are caused by, initiated by, or exacerbated by an underlying infection (e.g., a viral infection, bacterial infection, parasitic infection or fungal infection). For example, vasculitis is associated with acute, chronic or transient infections from viral, bacterial and fungal pathogens associated with symptoms of vascular inflammation. Accordingly, in certain embodiments, a method of treating a subject comprises a method of treating a latent, acute, chronic, or transient infection by a pathogen. Non-limiting examples of a pathogen includes a virus, a bacteria, a fungus and a parasite. Non-limiting examples of a virus include Hepatitis B virus (HBV), Hepatitis C virus (HCV), Epstein-Barr virus (EBV) and cytomegalovirus (hCMV).

In some embodiments, an autoimmune disorder or pro-inflammatory condition is not asthma, allergic asthma or an asthmatic condition. In some embodiments, an autoimmune disorder or pro-inflammatory condition is not a disorder or disease that includes asthma, allergic asthma or an asthmatic condition. In some embodiments, a method of treatment does not include treating a subject who has asthma, allergic asthma, an asthmatic condition or asthmatic bronchitis. In some embodiments, a method of treatment herein excludes treatment of a subject having or experiencing a chronic or acute condition selected from one or more of wheezing, shortness of breath, chest tightness, bronchoconstriction, coughing, pulmonary inflammation, atopic eczema, allergic rhinitis, asthma, allergic asthma, an asthmatic condition, asthmatic bronchitis and hay fever. In some embodiments, a method of treatment herein excludes treatment of a subject who has asthma, allergic asthma, an asthmatic condition or asthmatic bronchitis.

The term “subject” refers to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. A mammal can be a pregnant female. In certain embodiments, a mammal can be an animal disease model, for example, animal models used for the study of viral infections. In some embodiments, a subject is a human.

In some embodiments, a subject has or is suspected of having an autoimmune disorder, or pro-inflammatory condition. A diagnosis of an autoimmune disorder or pro-inflammatory condition can be made by a medical professional using any suitable method or criteria. In some embodiments, a subject is at risk of having or developing an autoimmune disorder or pro-inflammatory condition. For example, a subject may have a viral, bacterial, fungal or parasitic infection that is associated with, or suspected of causing a certain autoimmune disorder or pro-inflammatory condition. In some embodiments, a subject has, or is suspected of having a viral, bacterial, fungal or parasitic infection. In certain embodiments, a subject may have been exposed to a virus, bacteria, fungus or parasite that is associated with, or suspected of causing an autoimmune disorder or pro-inflammatory condition. Accordingly, a subject may be “at risk” of a viral infection, bacterial infection, fungal infection or parasite infection. Further, a subject may be at risk of having or developing an autoimmune disorder or pro-inflammatory condition.

In certain embodiments, a method of treatment comprises administering a therapeutically effective amount of an agent to the subject. The term “therapeutic agent” as used herein, collectively refers to an adenosine reuptake inhibitor, adenosine salvage pathway inhibitor, adenosine degradation pathway inhibitor, adenosine, deoxyadenosine, a variant or derivative thereof, a compound or composition that stimulates adenosine uptake, or a compound or composition that stimulates the adenosine salvage, reuptake, or degradation pathway.

In certain embodiments, a method of treatment comprises administering a therapeutically effective amount of adenosine reuptake inhibitor, adenosine salvage pathway inhibitor, adenosine degradation pathway inhibitor to the subject. Non-limiting examples of an adenosine reuptake inhibitor include acadesine, acetate, barbiturates, benzodiazepines, calcium channel blockers, carbamazepine, carisoprodol, cilostazol, cyclobenzaprine, dilazep, dipyridamole, estradiol, ethanol, flumazenil, hexobendine, hydroxyzine, indomethacin, inosine, KF24345, meprobamate, nitrobenzylthioguanosine, nitrobenzylthioinosine, papaverine, pentoxifylline, phenothiazines, phenytoin, progesterone, propentofylline, propofol, puromycin, r75231, soluflazine, toyocamycin, tracazolate, and tricyclic antidepressants. In some embodiments, an adenosine reuptake inhibitor is dipyridamole.

In certain embodiments, a method of treatment comprises administering a therapeutically effective amount of adenosine, deoxyadenosine, a variant or a derivative thereof to a subject. Non-limiting examples of derivatives of adenosine include N—[B-(5-chloro indolyl-3)-ethyl] adenosine, N—[B-(5-bromo indolyl-3)-ethyl] adenosine, N-[B—(S-methyl indolyl-3)-ethyl] adenosine, N—[B-(2-methyl indolyl-3)-ethyl] adenosine, N—[B—(S-phenoxy indolyl-3)-ethyl] adenosine, N-[fi-(5-acetyl indolyl-3)-ethyl] adenosine, N—[B-(5-mercapto indolyl-3)-ethyl] adenosine, N-[fi(5-methylmercapto indolyl-3)-ethyl] adenosine, N—[B—(S-nitrO indolyl-3)-ethyl] adenosine, N-[fi-(5-carboxy indolyl-3)-ethyl] adenosine, N-[13-(5-carboxyethyl indolyl-3)-ethyl] adenosine, N-[/3-(S-methylsulfonylamino indolyl-3)-ethyl] adenosine, N-[fi-(5-chloro imidazolyl-4)-ethyl] adenosine, N″—[B-(1-ethyl imidazolyl-4)-ethyl] adenosine, N—[B-(5-methyl imidazolyl-4)-ethyl] adenosine, N—[,6-(S-ethyl imidazolyl-4)-ethyl] adenosine, N—[B-(1-benzyl imidazolyl-4)-ethyl] adenosine, N—[B-(1-benzoyl imidazolyl-4)-ethyl] adenosine, N—[B-2-hydroxy-2(imidazolyl-4)-ethyl] adenosine, N-[fi-(2-methylmercapto imidazolyl-4)-ethyl] adenosine, N-[fi-(2-ethylmercapto imidazolyl-4)-ethyl] adenosine, N—IB—(Z-nitrO imidazolyl-4)-ethyl] adenosine, N—[B—(S-carboxy imidazolyl-4)-ethyl] adenosine, N—[B—(S-CarbOXymethyl imidazolyl-4)-ethyl]adenosine, imidazolyl-4)-ethyl1 N—[B—(Z-methyl-S-hydroxy-indolyl-3)-ethyl1 adenosine N—[[HZ-methyl-5-methoxy-indolyl-3)-ethyl] adenosine, N— B-(2-ethyl-indolyl-3)-ethyl] adenosine, N—[B-(2-ethyl-5-hydroxy-indolyl)3-ethyl1 adenosine, and N—[B-(2-ethyl-5-methoxy-indolyl-3)-ethyl] adenosine.

Also, presently provided are methods of treating a subject having or suspected of having a disease or disorder by administering an analogue of adenosine, deoxyadenosine, a variant or derivative thereof, to the subject. In certain embodiments, the method comprises treating an autoimmune disorder, or pro-inflammatory condition comprising administering a therapeutically effective amount of an analogue of adenosine, deoxyadenosine, a variant or derivative thereof, to the subject. In such embodiments, the analogue inhibits the activity of adenosine, or deoxyadenosine.

In other embodiments, the method comprises treating a subject having a neoplasia, neoplastic disorder or cancer comprising administering a therapeutically effective amount of adenosine, deoxyadenosine, or a variant or derivative thereof, or an analogue of adenosine or deoxyadenosine to the subject. In such embodiments, the analogue of adenosine or deoxy adenosine mimics or agonizes the biological effects of adenosine, or deoxyadenosine.

Disclosed herein are methods and uses of treating a subject having or suspected of having a neoplasia, neoplastic disorder, tumor, cancer or malignancy. A method of treating a neoplasia, neoplastic disorder, tumor, cancer or malignancy, in some embodiments, comprises administering a compound or composition that stimulates adenosine uptake or the adenosine salvage or degradation pathway(s). In certain embodiments, a method herein comprises contacting a cell of a subject with a compound or composition that stimulates adenosine uptake or the adenosine salvage or degradation pathway(s) disclosed herein. In some embodiments, a method comprises reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy. In some embodiments, a method comprising inhibiting or reducing relapse or progression of a neoplasia, neoplastic disorder, tumor, cancer or malignancy. Non-limiting examples of a neoplasia, neoplastic disorder, tumor, cancer or malignancy include: a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma. A neoplasia, neoplastic disorder, tumor, cancer or malignancy may comprise or involve hematopoietic cells. Non-limiting examples of a sarcoma include a lymphosarcoma, liposarcoma, osteosarcoma, chondrosarcoma, leiomyosarcoma, rhabdomyosarcoma or fibrosarcoma. In some embodiments, a neoplasia, neoplastic disorder, tumor, cancer or malignancy is a myeloma, lymphoma or leukemia. In some embodiments, a neoplasia, neoplastic disorder, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer. In some embodiments, a neoplasia, neoplastic disorder, tumor, cancer or malignancy comprises a small cell lung or non-small cell lung cancer. In some embodiments, a neoplasia, neoplastic disorder, tumor, cancer or malignancy comprises a stem cell neoplasia, tumor, cancer or malignancy.

In certain embodiments a neoplasia, neoplastic disorder, tumor, cancer or malignancy comprises a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma. In some embodiments, a neoplasia, neoplastic disorder, tumor, cancer or malignancy comprises hematopoietic cells. In some embodiments, a sarcoma comprises a lymphosarcoma, liposarcoma, osteosarcoma, chondrosarcoma, leiomyosarcoma, rhabdomyosarcoma or fibrosarcoma. In some embodiments, a neoplasia, neoplastic disorder, tumor, cancer or malignancy comprises a myeloma, lymphoma or leukemia. In some embodiments, a neoplasia, neoplastic disorder, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer. In some embodiments, a lung neoplasia, neoplastic disorder, tumor, cancer or malignancy comprises small cell lung or non-small cell lung cancer.

In some embodiments, a method of treatment results in partial or complete destruction of the neoplastic, tumor, cancer or malignant cell mass; a reduction in volume, size or numbers of cells of the neoplastic, tumor, cancer or malignant cell mass; stimulating, inducing or increasing neoplastic, tumor, cancer or malignant cell necrosis, lysis or apoptosis; reducing neoplasia, tumor, cancer or malignancy cell mass; inhibiting or preventing progression or an increase in neoplasia, tumor, cancer or malignancy volume, mass, size or cell numbers; or prolonging lifespan. In some embodiments, a method of treatment results in reducing or decreasing severity, duration or frequency of an adverse symptom or complication associated with or caused by the neoplasia, tumor, cancer or malignancy. In some embodiments, a method of treatment results in reducing or decreasing pain, discomfort, nausea, weakness or lethargy. In some embodiments, a method of treatment results in increased energy, appetite, improved mobility or psychological well-being.

In some embodiments, a method comprises administering an anti-cell proliferative, anti-neoplastic, anti-tumor, anti-cancer or immune-enhancing treatment or therapy in combination with a compound or composition that stimulates adenosine uptake or the adenosine salvage pathway.

In certain embodiments, a pharmaceutical composition comprises therapeutic agent. In certain embodiments, a pharmaceutical composition comprises an adenosine reuptake inhibitor, adenosine salvage pathway inhibitor or adenosine degradation pathway inhibitor, adenosine, deoxyadenosine, a variant or derivative thereof, or a compound or composition that stimulates adenosine uptake or a compound or composition that stimulates the adenosine salvage or degradation pathway(s). A pharmaceutical composition can be formulated for a suitable route of administration. In some embodiments, a pharmaceutical composition is formulated for subcutaneous (s.c.), intradermal, intramuscular, intraperitoneal and/or intravenous (i.v.) administration. In certain embodiments, a pharmaceutical composition can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants (Remington's Pharmaceutical Sciences, 18th Ed., A. R. Gennaro, ed., Mack Publishing Company (1995)).

In certain embodiments, a pharmaceutical composition comprises a suitable excipient, non-limiting example of which include anti-adherents (e.g., magnesium stearate), a binder, fillers, monosaccharides, disaccharides, other carbohydrates (e.g., glucose, mannose or dextrins), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweeteners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington's Pharmaceutical Sciences, 18th Ed., A. R. Gennaro, ed., Mack Publishing Company (1995). The term “binder” as used herein refers to a compound or ingredient that helps keeps a pharmaceutical mixture combined. Suitable binders for making pharmaceutical formulations and are often used in the preparation of pharmaceutical tablets, capsules and granules are known to those skilled in the art. In some embodiments, a pharmaceutical composition comprises a binder.

In some embodiments, a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agent includes those having a sulfhydryl group such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments, a pharmaceutical composition comprises one or more suitable preservatives. Non-limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments, a pharmaceutical composition is free of preservatives.

In some embodiments a composition, pharmaceutical composition, adenosine reuptake inhibitor, adenosine salvage pathway inhibitor or adenosine degradation pathway inhibitor, adenosine, deoxyadenosine, a variant or derivative thereof, or a compound or composition that stimulates adenosine uptake or a compound or composition that stimulates the adenosine salvage or degradation pathway(s) is substantially free of blood, or a blood product contaminant (e.g., blood cells, platelets, polypeptides, minerals, blood borne compounds or chemicals, and the like). In some embodiments a composition, pharmaceutical composition or therapeutic agent is substantially free of serum and serum contaminants (e.g., serum proteins, serum lipids, serum carbohydrates, serum antigens and the like). In some embodiments a composition, pharmaceutical composition or therapeutic agent is substantially free a pathogen (e.g., a virus, parasite or bacteria). In some embodiments a composition, pharmaceutical composition or therapeutic agent is substantially free of endotoxin. In some embodiments a composition, pharmaceutical composition or therapeutic agent is sterile. In certain embodiments, a composition or pharmaceutical composition comprises a therapeutic agent and a diluent (e.g., phosphate buffered saline). In certain embodiments, a composition or pharmaceutical composition comprises a therapeutic agent and an excipient, (e.g., sodium citrate dehydrate, or polyoxyethylene-sorbitan-20 mono-oleate (polysorbate 80)).

The pharmaceutical compositions described herein may be configured for administration to a subject in any suitable form and/or amount according to the therapy in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents. In some embodiments, a pharmaceutical composition suitable for parental administration may contain one or more excipients. In some embodiments, a pharmaceutical composition is lyophilized to a dry powder form. In some embodiments, a pharmaceutical composition is lyophilized to a dry powder form, which is suitable for reconstitution with a suitable pharmaceutical solvent (e.g., water, saline, an isotonic buffer solution (e.g., PBS), and the like). In certain embodiments, reconstituted forms of a lyophilized pharmaceutical composition are suitable for parental administration (e.g., intravenous administration) to a mammal.

In some embodiments, pharmaceutical compositions described herein may be configured for topical administration and may include one or more of a binding and/or lubricating agent, polymeric glycols, gelatins, cocoa-butter or other suitable waxes or fats. In some embodiments, a pharmaceutical composition described herein is incorporated into a topical formulation containing a topical carrier that is generally suited to topical drug administration and comprising any suitable material known to those skilled in the art. In certain embodiments, a topical formulation of a pharmaceutical composition is formulated for administration of a therapeutic agent from a topical patch.

In certain embodiments, an optimal pharmaceutical composition will be determined by one skilled in the art depending upon, for example, the intended route of administration, delivery format and desired dosage (see e.g., Remington's Pharmaceutical Sciences, supra). In certain embodiments, such compositions may influence the physical state, stability, rate of in vivo release and rate of in vivo clearance of the antibodies of the invention.

In some embodiments a composition, pharmaceutical composition or therapeutic agent described herein is used to treat a subject having or suspected of having an autoimmune disease, a pro-inflammatory condition, a pathogen infection, a neoplastic disorder or cancer. In certain embodiments, a therapeutic agent or pharmaceutical composition described herein is used in treating a neoplastic disorder or cancer in a subject. In some embodiments, disclosed herein is a method of treating a subject having or suspected of having a neoplastic disorder or cancer. In certain embodiments, a method of treating a subject having or suspected of having a neoplastic disorder or cancer comprises administering a therapeutically effective amount of a composition, pharmaceutical composition or therapeutic agent described herein to the subject. In certain embodiments, a method of treatment comprises contacting a cell (e.g., one or more cells) of a subject with a therapeutically effective amount of a composition, pharmaceutical composition or therapeutic agent described herein. A cell of a subject may be found inside a subject (e.g., in vivo) or outside the subject (e.g., in vitro or ex vivo).

A composition, pharmaceutical composition or therapeutic agent disclosed herein can be used to treat a suitable disease or condition disclosed herein. Any suitable method of administering a composition, pharmaceutical composition or therapeutic agent to a subject can be used. The exact formulation and route of administration for a composition for use according to the methods of the invention described herein can be chosen by the individual physician in view of the patient's condition. See, e.g., Fingl et al. 1975, in “The Pharmacological Basis of Therapeutics,” Ch. 1, p. 1; which is incorporated herein by reference in its entirety. Any suitable route of administration can be used for administration of a pharmaceutical composition or a therapeutic agent described herein. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments, a composition herein is provided to a subject. A composition that is provided to a subject is sometimes provided to a subject for self-administration or for administration to a subject by another (e.g., a non-medical professional). For example, a composition described herein can be provided as an instruction written by a medical practitioner that authorizes a patient to be provided a composition or treatment described herein (e.g., a prescription). In another example, a composition can be provided to a subject where the subject self-administers a composition orally, intravenously or by way of an inhaler, for example.

Alternately, one can administer compositions for use according to the methods of the invention in a local rather than systemic manner, for example, via direct application to the skin, mucous membrane or region of interest for treating, including using a depot or sustained release formulation.

In some embodiments, a pharmaceutical composition comprising a therapeutic agent can be administered alone (e.g., as a single active ingredient (AI or e.g., as a single active pharmaceutical ingredient (API)). In other embodiments, a pharmaceutical composition comprising a therapeutic agent can be administered in combination with one or more additional AIs/APIs, for example, as two separate compositions or as a single composition where the one or more additional AIs/APIs are mixed or formulated together with the therapeutic agent in a pharmaceutical composition.

A pharmaceutical composition can be manufactured by any suitable manner, including, e.g., by means of conventional mixing, dissolving, granulating, dragee-making, levigating, emulsifying, encapsulating, entrapping or tableting processes.

In some embodiments, a pharmaceutical composition comprising a therapeutic agent is administered at a suitable frequency or interval as needed to obtain an effective therapeutic outcome. An effective therapeutic outcome can be determined by monitoring the number, viability, growth, mitosis, or metastasis of neoplastic or cancerous cells in a subject affected with a neoplastic disorder or cancer. Accordingly, in certain embodiments, a decrease in the number, viability, growth, mitosis, or metastasis of neoplastic or cancerous cells in a subject is considered an effective therapeutic outcome. In some embodiments, a pharmaceutical composition comprising a therapeutic agent can be administered hourly, once a day, twice a day, three times a day, four times a day, five times a day, and/or at regular intervals, for example, every day, every other day, three times a week, weekly, every other week, once a month and/or simply at a frequency or interval as needed or recommended by a medical professional.

In some embodiments, an amount of a therapeutic agent in a composition is an amount needed to obtain an effective therapeutic outcome. In certain embodiments, the amount of a therapeutic agent in a composition (e.g., a pharmaceutical composition) is an amount sufficient to prevent, treat, reduce the severity of, delay the onset of, and/or alleviate a symptom of a neoplastic disorder or cancer, as contemplated herein.

A “therapeutically effective amount” means an amount sufficient to obtain an effective therapeutic outcome and/or an amount necessary sufficient to prevent, treat, reduce the severity of, delay the onset of, and/or alleviate a symptom of a neoplastic disorder or cancer. In certain embodiments, a “therapeutically effective amount” means an amount sufficient to terminate the grow of, and/or slow the growth of a neoplasm or cancer. In certain embodiments, a “therapeutically effective amount” means an amount sufficient to inhibit the replication of, and/or induce the death of one or more neoplastic cells. Determination of a therapeutically effective amount is well within the capability of those skilled in the art, especially in light of the detailed disclosure provided herein.

In some embodiments, an amount of a therapeutic agent in a composition is an amount that is at least a therapeutically effective amount and an amount low enough to minimize unwanted adverse reactions. The exact amount of a therapeutic agent or combinations of active agents required will vary from subject to subject, depending on age, weight, and general condition of a subject, the severity of the condition being treated, and the particular combination of drugs administered. Thus, it is not always possible to specify an exact therapeutically effective amount to treat a neoplastic disorder in a diverse group of subjects. As is well known, the specific dosage for a given patient under specific conditions and for a specific disease will routinely vary, but determination of the optimum amount in each case can readily be accomplished by simple routine procedures. Thus, a therapeutically effective amount of a therapeutic agent used to treat a neoplastic disorder may be determined by one of ordinary skill in the art using routine experimentation.

In certain embodiments, an amount of a therapeutic agent in a composition is administered at a suitable therapeutically effective amount or a dose (e.g., at a suitable volume and concentration, which sometimes depends, in part, on a particular route of administration). Within certain embodiments, a therapeutic agent (e.g., a therapeutic agent in a composition) can be administered at a dose from about 0.0001 mg/kg (e.g., per kg body weight of a subject) to 500 mg/kg, 0.001 mg/kg to 500 mg/kg, 0.001 mg/kg to 500 mg/kg, 0.01 mg/kg to 500 mg/kg, 0.1 mg/kg to 500 mg/kg, 0.1 mg/kg to 400 mg/kg, 0.1 mg/kg to 300 mg/kg, 0.1 mg/kg to 200 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg or 0.1 mg/kg to 1 mg/kg. In some aspects the amount of a therapeutic agent can be about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg. In some embodiments, a therapeutically effective amount of a therapeutic agent is between about 0.1 mg/kg to 500 mg/kg, or between about 1 mg/kg and about 300 mg/kg. Volumes suitable for intravenous administration are well known.

Within certain embodiments, a therapeutic agent (e.g., a therapeutic agent in a composition) is administered at a dose from about 0.000001 mg to 500 mg, 0.001 mg to 500 mg, 0.001 mg to 500 mg, 0.01 mg to 500 mg, 0.1 mg to 500 mg, 0.1 mg to 400 mg, 0.1 mg to 300 mg, 0.1 mg to 200 mg, 0.1 mg to 150 mg, 0.1 mg to 100 mg, 0.1 mg to 75 mg, 0.1 mg to 50 mg, 0.1 mg to 25 mg, 0.1 mg to 10 mg, 0.1 mg to 5 mg or 0.1 mg to 1 mg. In some aspects the amount of a therapeutic agent can be about 10 mg, 9 mg, 8 mg, 7 mg, 6 mg, 5 mg, 4 mg, 3 mg, 2 mg, 1 mg, 0.9 mg, 0.8 mg, 0.7 mg, 0.6 mg, 0.5 mg, 0.4 mg, 0.3 mg, 0.2 mg, or 0.1 mg. In some embodiments, a therapeutically effective amount of a therapeutic agent is between about 0.1 μg and 10,000 μg, between about 0.1 μg and 1000 μg, between about 0.1 μg and 500 μg, or between about 0.1 μg and 100 μg.

In certain embodiments of the present invention, a method or pharmaceutical composition disclosed herein is specifically directed, applied or targeted to endothelial cells or specifically modulates the innate immune response.

A pharmaceutical composition comprising an amount or dose of a therapeutic agent can, if desired, be provided in a kit, pack or dispensing device, which can contain one or more doses of a therapeutic agent. The pack can for example comprise metal or plastic foil, such as a blister pack. The pack or dispenser device can be accompanied by instructions for administration. The pack or dispenser can also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert.

In some embodiments, a kit or pack comprises an amount of a therapeutic agent sufficient to treat a patient for 1 day to 1 year, 1 day to 180 days, 1 day to 120 days, 1 day to 90 days, 1 day to 60 days, 1 day to 30 days, or any day or number of days there between, 1-4 hours, 1-12 hours, or 1-24 hours.

A kit optionally includes a product label or packaging inserts including a description of the components or instructions for use in vitro, in vivo, or ex vivo, of the components therein. Exemplary instructions include instructions for a diagnostic method, treatment protocol or therapeutic regimen. In certain embodiments, a kit comprises packaging material, which refers to a physical structure housing components of the kit. The packaging material can maintain the components sterilely, and can be made of material commonly used for such purposes (e.g., paper, corrugated fiber, glass, plastic, foil, ampules, vials, tubes, etc.). Product labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards. Product labels or inserts can include identifying information of one or more components therein, dose amounts, clinical pharmacology of the active ingredient(s) including mechanism of action, pharmacokinetics (PK) and pharmacodynamics (PD). Product labels or inserts can include information identifying manufacturer information, lot numbers, manufacturer location, date, information on an indicated condition, disorder, disease or symptom for which a kit component may be used. Product labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include labels or instructions for practicing any of the methods and uses of the invention described herein. Product labels or inserts can include information on potential adverse side effects and/or warnings.

In some embodiments, a kit is a diagnostic kit comprising a therapeutic agent. A therapeutic agent comprised in a diagnostic kit can take any suitable form. In some embodiments, a diagnostic comprises a therapeutic agent and a detectable label. In certain embodiments, for example, a diagnostic kit comprises or consists of a stick test, including necessary reagents to perform the method of the invention and to produce, for example, a colorimetric result which can be compared against a color chart or standard curve.

### Example 1

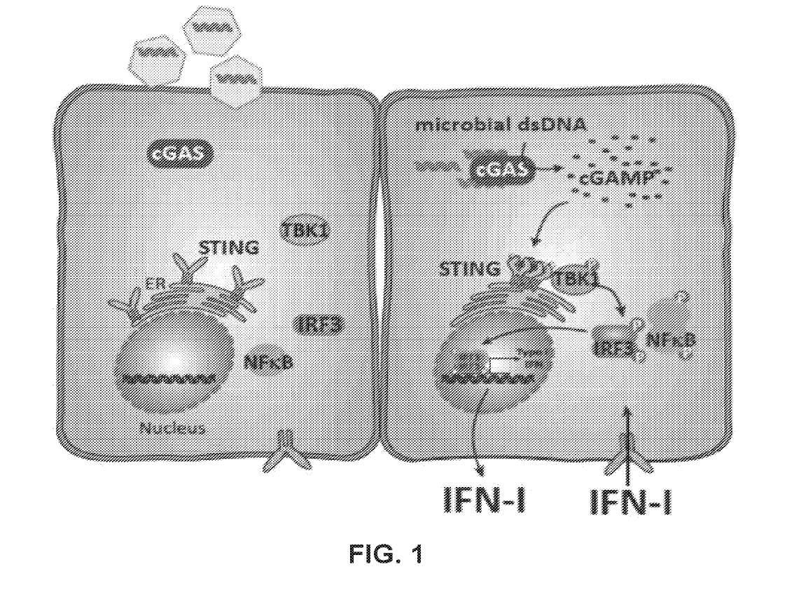

A primary part of innate immunity involves induction of pro-inflammatory cytokines, including type I Interferons (IFN-I, IFN—α, and IFN—β), which recruit and activate immune cells. High levels of IFN-I are produced via direct sensing of an array of viral, bacterial or neoplastic antigens, which are innate ligands. For example, viral double stranded DNA genomes are sensed in the cytoplasm of infected cells (see FIG. 1). The DNA binding protein cGAS catalyzes the formation of the cyclic di-nucleotide second messenger cGAMP, which binds and activates the innate effector protein STING. STING recruits a kinase TBK1 to phosphorylate the transcription factor IRF3, a crucial component of the IFN—β transcriptional enhanceosome in the nucleus.

Cells harbor many pathways for inducing IFN-I, and the highly conserved cGAS-STING-IFN-I axis has emerged as a universal trigger of the response to DNA-encoding viruses, including HBV, CMV, EBV and Parvovirus. Activation of this pro-inflammatory cascade is also linked to auto-immune diseases such as systemic lupus erythematosus, Aicard-Goutières Syndrome and Psoriasis, which are characterized by vasculitis. Furthermore, gain-of-function mutations in STING cause STING-associated vasculopathy with onset in infancy (SAVI), characterized by high circulating levels of IFN-I, chronically activated immune cells, and inflamed blood vessels. These data strongly suggest that cell-intrinsic cGAS-STING-IFN-I signaling plays an important role in vasculitis.

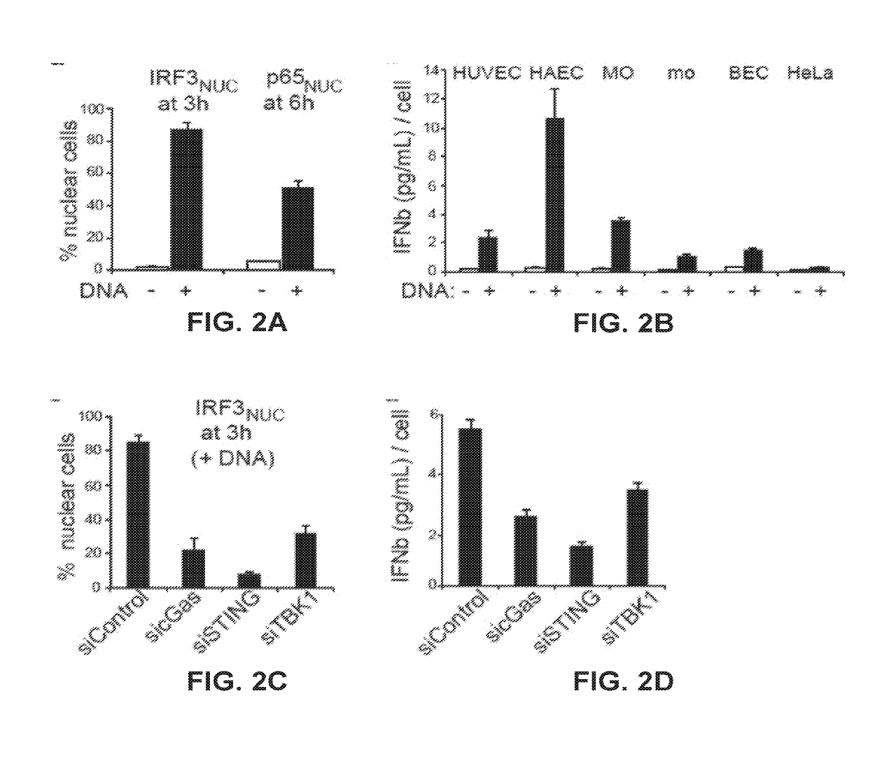

Primary human endothelial cells display robust cGAS-STING-IFN-I signaling. Primary human vascular endothelial cells mount remarkably robust innate immune responses to DNA and DNA viruses (i.e. CMV) through the cGAS-STING-IFN-I pathway. To quantify IFN-I pathway activation in single cells, the inventors measured inducible nuclear translocation of endogenous IRF3 in cells, using an automated, micro-scaled in situ immunofluorescence assay. Transfection of 1 ug/mL double stranded poly dA:dT DNA into HUVEC induced robust IRF3 translocation after 3 h (FIG. 2a) and IFN-b production at 16 h (FIG. 2b). Notably, transfection of siRNAs targeting cGAS, STING or TBK1 into HUVEC, which typically depletes>80% of target protein levels, significantly diminished IRF3 activation and IFN—β mRNA induction (FIGS. 2c and 2d). Similar results were obtained after infection with DNA-containing viruses such as CMV.

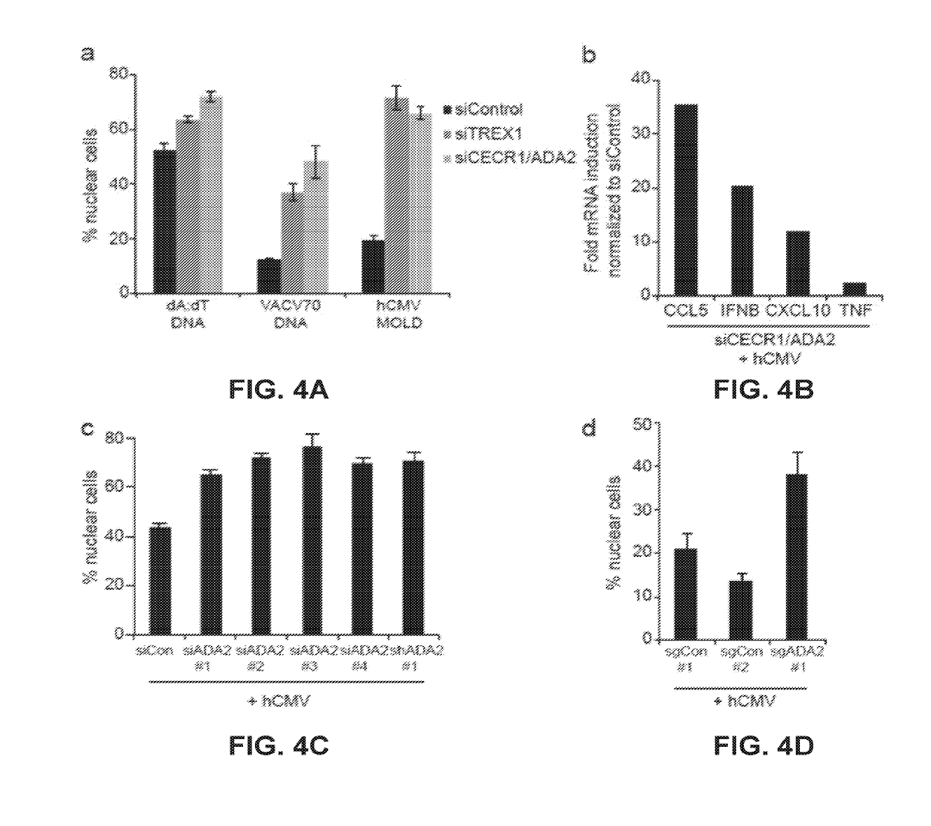

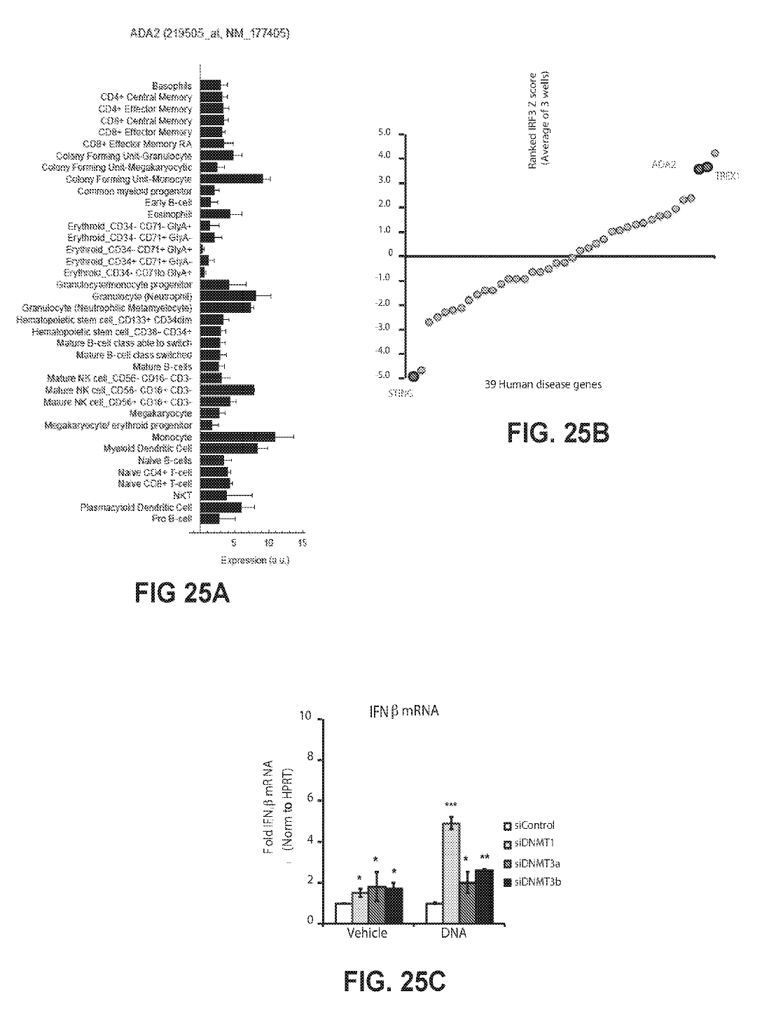

Genome-scale RNAi in vascular endothelial cells identifies ADA2 as a new negative regulator of cGAS-STING-IFN-I signaling: The inventors use unbiased loss-of-function approaches to uncover new regulatory targets in immune signaling pathways (9, 10, 20, 21). The inventors performed genome-scale RNAi experiments in HUVEC to monitor IRF3 translocation after stimulation with immuno-stimulatory DNA to activate the cGAS-STING-IFN-I pathway. The averaged IRF3 translocation value associated with each gene-specific siRNA was ranked using a normalized Z score, resulting in identification of 39 gene activators that significantly increased DNA-induced IRF3 translocation (FIG. 25b), without significantly affecting cell growth, viability or morphology. Among these candidates were expected genes whose depletion decreased IRF3 activation. The PAN vasculitis susceptibility gene ADA2 was identified as a new inhibitor of the pathway (FIG. 4). Several different ADA2-specific siRNAs enhanced IRF3 translocation and IFN—β induction, as assessed by Taqman qRT-PCR, after DNA transfection or CMV infection (FIGS. 4a and 4b). Similar results were observed for other innate immune cytokines such as IL6, CCL5 and TNF.

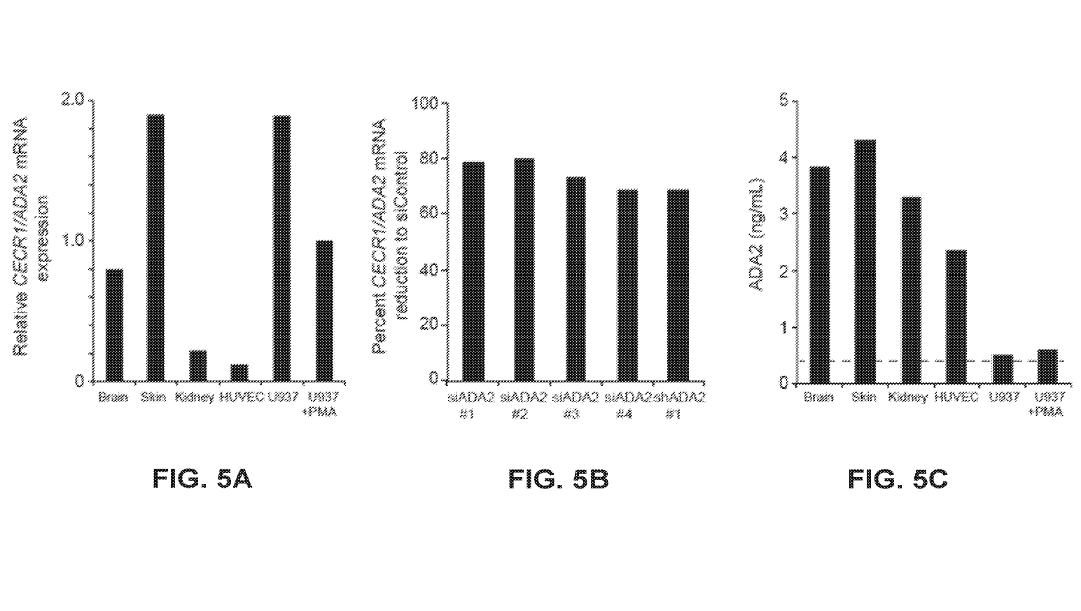

ADA2 is expressed in primary human vascular endothelial cells: ADA2 is a secreted enzyme highly expressed in monoyctes. It is thought in the art that ADA2 levels in HUVEC endothelial cells is low, however, the present invention provides that RNAi depletion of endothelial ADA2 elicits significant effects on the anti-viral innate immune responses. To confirm ADA2 expression in primary human endothelial cells, the inventors performed Taqman qRT-PCR and ELISA experiments. ADA2 mRNA expression was detected in umbilical vein, brain, kidney and skin endothelial cells, using U937 monocytes as a positive control (FIG. 5a). In HUVEC, transfection of ADA2 siRNAs induced>80% knockdown of the mRNA (data not shown). Similarly, ADA2 protein was positively quantified in supernatants from endothelial cells and monocytes by ELISA (FIG. 5b). These results clearly demonstrate that endothelial cells express ADA2.

The present invention provides that depletion of ADA2 in primary human vascular endothelial cells enhances cell-intrinsic innate immune responses to virus infection, through increased production of pro-inflammatory cytokines such as IFN—0, IL-6 and TNF.

### Example 2

### Research Design, Methods & Analysis

The inventors performed siRNA experiments in HUVEC which achieved>80 depletion of ADA2 mRNA, thereby precipitating a significant effect on induction of innate immune cytokines induced by virus infection. To better effectively model PAN vasculitis and ADA2 deficiency, knock-out endothelial cells were generated using the CRISPR/Cas9 targeted genome editing strategy which successfully generated STING-deficient endothelial cells. As most PAN mutations essentially cause loss of ADA2 protein expression, CRISPR/Cas9 was used to introduce premature termination within the first coding exon of the CECR1/ADA2 gene. In addition to empty vector targeted cells, IRF3-deficient cells were produced as a control for defective IFN-I induction. Briefly, hTERT-immortalized HUVEC were transduced with lentivirus encoding Cas9 and a gRNA targeting the first coding exon of the target gene. Next, cells were selected with puromycin and sorted into single-cell populations. Loss of ADA2 gene and protein expression was verified by Sanger sequencing and western blotting.

To characterize cell-intrinsic innate immune responses in control, ADA2 and IRF3 knock-out cells, a time course of IFN—β gene and protein induction (0-24 h) was performed using qRT-PCR and ELISA. Other highly inducible innate immune genes were also monitored by qRT-PCR and western blot/ELISA, including IP-10, IL6, RANTES, IL1b, IFIT2 and MX2. Cells were stimulated with an array of well-characterized innate immune stimulants/ligands: 1) Cytoplasmic nucleic acids (0.2-1.0 ug/mL of transfected poly dA:dT DNA, 2'S′-cGAMP, sonicated tumor DNA and poly I:C RNA, available from Invivogen); 2) Nod-like receptor ligands (0.2-1.0 ug/mL iE-DAP and MDP, from Invivogen); and 3) Toll-like receptor ligands (various effective concentrations of CpG DNA, LPS, flagellin, R848 and extracellular poly I:C, from Invivogen).

To further analyze innate immune responses to infection, ADA2-deficient cells were infected with CMV or EBC strain B958 (available from ATCC) at MOI=1, 2, 5 and 10, for early time points so as to minimize cell death (3-6 h). In addition to measuring IFN—β induction, the acute replication levels of each virus were assessed, using flow cytometry for UL44 antigen (CMV) and EBV-VCA antigen (EBV). Together, these experiments provide an in-depth analysis of the cell-intrinsic innate immune responses of ADA2-deficient cells to a panel of innate immune antigens as well as PAN-associated viruses such as CMV and EBV.

Secreted ADA2 acts as a growth factor for monocyte differentiation and as an enzyme that metabolizes extracellular adenosine, a potently immune-modulatory nucleoside. To explore the mechanism by which ADA2 dampens anti-viral innate immune responses in endothelial cells, each function of ADA2 (i.e. enzymatic vs non-enzymatic) was first examined. First, the importance of the catalytic activity of ADA2 was determined by performing a “rescue” experiment by adding back active recombinant ADA2 (commercially available from Sigma) or heat and pH-inactivated ADA2, and then anti-viral innate immune responses were assessed. Next, the effect of extracellular adenosine was examined by pre-treating umbilical cord, brain, skin and kidney endothelial cells with increasing concentrations of extracellular adenosine (in the uM range of concentration) for up to 72 h. Cells pre-treated with adenosine were stimulated with DNA or infected with CMV, and innate gene expression is analyzed by qRT-PCR and ELISA/western. Without being limited to any particular theory, as the enhanced innate responses in ADA2-deficient endothelial cells involve vascular catalytic activity of ADA2 and accumulation of extracellular adenosine, the present invention provides that extracellular adenosine supplementation enhances the expression of IFN—β, IL-6 and TNF. Other metabolites and pharmacological activators or inhibitors of the adenosine pathway were also examined: i) extracellular deoxyadenosine, another substrate for deamination by ADA2; ii) NECA, a non-hydrolyzable analogue of adenosine; iii) pentostatin, a specific inhibitor of ADA2; iv) EHNA, a specific inhibitor of ADA1, the intracellular adenosine deaminating enzyme. RNAi-mediated depletion of ADA1 and all adenosine receptor and transporter isoforms expressed in endothelial cells was also performed.

In order to examine methods aimed at suppressing pro-inflammatory responses in ADA2-deficient cells, the importance of the key signaling and effector molecules operating in the cGAS-STING-IFN-I pathway (FIG. 1) was first confirmed by depleting cGAS, STING, TBK1, IRF3 or NFkB p65 in control and ADA2-deficient cells and the effects on innate gene expression induced by DNA or DNA virus infection (CMV) was examined Knockdown of these genes either abolishes or significantly diminishes innate gene expression in both wild type and ADA2-deficient cells infected with CMV or treated with DNA antigens. Next, the utility of anti-viral drugs in curbing pro-inflammatory responses in virally-infected endothelium was examined. The ability of anti-CMV drugs gancyclovir, foscarnet (which also efficiently inhibits the replication of EBV) and cidofovir in limiting viral replication of CMV in wild type and ADA2-deficient cells was determined. Notably, the innate immune responses and pro-inflammatory cytokine expression in the absence and presence of anti-viral drugs was assessed. Since viral DNA replication is the main trigger of cGAS-STING-IFN-I pathway activation, the therapeutic inhibitor of viral replication serves two clinically beneficial purposes in ADA2-deficient endothelial cells: 1) limit cellular damage, and ii) limit inflammation.

### Example 3

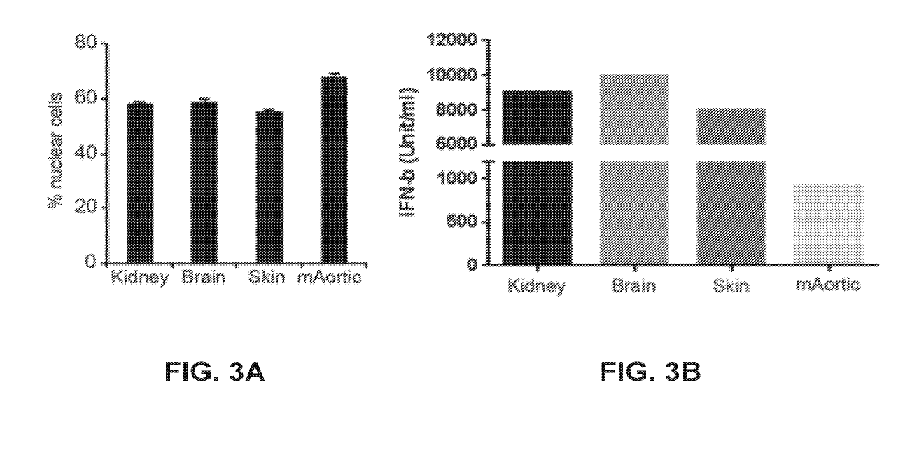

Primary human endothelial cells are a robust cellular model for studying anti-viral STING-IFN-I signaling: The inventors have previously shown that primary human vascular endothelial cells (i.e. HUVEC) mount remarkably robust cell-intrinsic innate immune responses through the STING-IFN-I pathway upon infection with hCMV clinical strain MOLD. To reliably quantify IFN-I pathway activation in single cells, inducible nuclear translocation of endogenous IRF3 was measured using an automated, micro-scaled immunofluorescence assay (3, 12, 18, 19,22). Transfection of 1 ug/mL of double stranded DNA(poly dA:dT, Invivogen) into HUVEC induces robust IRF3 and NFκB p65 translocation to the nucleus after 3 h (FIG. 2a), and IFNβ production after 16 h, as measured by protein ELISA (FIG. 2b). Transfection of siRNAs targeting cGAS, STING or TBK1, which deplete>80% of target protein levels, significantly diminishes IRF3 translocation and IFNβ induction, as measured by Taqman qRT-PCR after 4 h (FIGS. 2c and 2d). Similar results for IRF3 and IFNβ were obtained using other DNA ligands (data not shown): i) interferon stimulatory DNA, a non-CpG oligomer from the L. monocytogenes genome; ii) VACV70 from vaccinia virus; iii) HSV-60 from Herpes Simplex Virus 1 (HSV1), and iv) infection with HSV1 or hCMV. Robust cell-intrinsic IRF3 and IFNβ responses to DNA and hCMV were also detected in cultured primary vascular endothelial cells isolated from the brain, kidney, skin and aorta as well as mouse aortic endothelial cells (FIGS. 3a and 3b, DNA at 3 h post infection). These data show that primary vascular endothelial cells mount robust cell-intrinsic innate responses via STING.

The PAN/DADA2 gene CECR1/ADA2 is a negative regulator of STING-IFN-I signaling. To specifically examine the link between endothelial STING signaling and clinical vasculitis, the inventors performed RNAi and CRISPR/Cas9 experiments in HUVEC cells to assess the role of known human vasculitis disease genes in IRF3 activation and IFNβ production after DNA treatment or DNA virus infection (hCMV). By searching the Online Mendelian Inheritance in Man (OMIM) online database, 37 human vasculitis genes associated with 45 distinct syndromes associated were identified, including TREX1 (AGS1 vasculitis) and STING (SAVI vasculitis). The inventors depleted each vasculitis-associated gene using RNAi and/or CRISPR/Cas9, and then examined IRF3/IFNβ activation in response to DNA treatment or infection with hCMV clinical strain MOLD, which was shown to productively infect vascular endothelial cells with high efficiency (FIG. 3). siRNA depletion of the PAN/DADA2 vasculitis gene CECR1/ADA2 significantly enhanced IRF3 activation using a relatively low-dose of DNA treatment (e.g. poly dA:dT and VACV70, 100-200 ng/mL for 3 h) or after hCMV infection (FIG. 4a, MOI=5 for 4 h), similar in magnitude to increases observed after TREX1 depletion. Depletion of ADA2 also enhanced transcription of pro-inflammatory cytokine genes IFNβ, IL6, CCL5 and TNF in response to DNA treatment and hCMV infection, as detected by TaqMan qRT-PCR (FIG. 4b, hCMV shown). Effects of ADA2 depletion recapitulated using several different ADA2-specific siRNA and shRNA sequences (FIG. 4c). Furthermore, preliminary experiments using CRISPR/Cas9 in HUVEC showed that ADA2-specific sgRNA also enhanced IRF3 responses after hCMV infection (FIG. 4d). Together, these experiments demonstrate that deficiency of ADA2 in primary endothelial cells results in enhanced cell intrinsic innate immune responses to virus.

ADA2 mRNA and protein are expressed in primary human vascular endothelial cells: ADA2 is a secreted enzyme that is highly expressed by myeloid cells. Although a previous study reported relatively low ADA2 expression in HUVEC, the results described herein clearly show that multiple, distinct siRNA, shRNA and sgRNA sequences targeting ADA2 elicited effects on innate immune responses in HUVEC, strongly arguing against the likelihood of “off-target” effects of RNAi or CRISPR. To assess mRNA and protein expression of ADA2 in primary human endothelial cells, the inventors performed Taqman qRT-PCR and protein ELISA experiments. ADA2 mRNA was detected in human endothelial cells isolated from umbilical vein, brain, kidney and skin (commercially obtained from Lonza), using the U937 monocytic cell line as a positive control (FIG. 5a). Notably, all ADA2-specific siRNAs or shRNAs used in the functional assays for IRF3 and IFNβ activation (FIGS. 4a-c) induced robust mRNA depletion in HUVEC (˜75-80%) (FIG. 5b). ADA2 protein was also positively quantified in culture supernatants from endothelial cells or U937 monocytes using protein ELISA (FIG. 5c). Secreted ADA2 protein levels were higher in the endothelial cell supernatants compared to the monocyte supernatants, which, without being bound to any particular theory, is because monocytes are known to store intracellular pools of ADA2. Together, the data provided herein show that ADA2 is abundantly expressed in primary human vascular endothelial cells at the mRNA and protein levels.

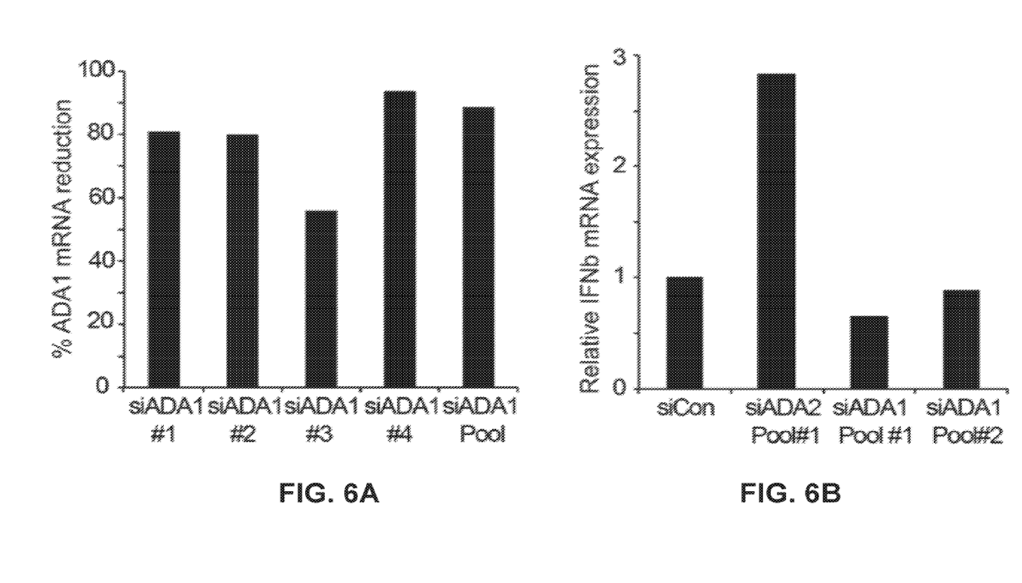

In humans, two enzymes deaminate Ado/dAdo, ADA1 (intracellular) and ADA2 (extracellular). Patients harboring loss of function mutations in ADA1 exhibit profound severe combined immune deficiency (SCID) in the absence of vascular complications, while patients deficient in ADA2 exhibit auto-inflammation with some defects in immune function. To study the role of ADA1 in cell-intrinsic innate immune responses in endothelial cells, the inventors used siRNAs to efficiently deplete ADA1 in HUVEC (FIG. 6a). In contrast to deficiency of ADA2, loss of ADA1 expression did not increase IRF3 nuclear translocation or IFNβ production in response to DNA stimulation (VACV70, poly dA:dT) or hCMV infection (FIG. 6b, IFNβ induction by hCMV shown). This data shows that deficiency of ADA2 but not ADA1 drives enhanced pro-inflammatory responses in endothelial cells, in accordance with clinical phenotypes observed in human patients.

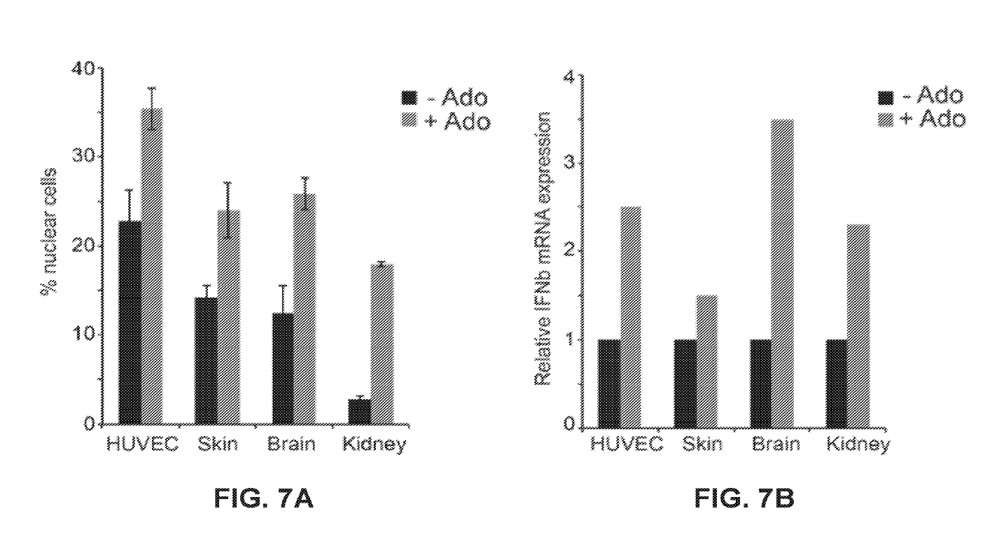

Extracellular Ado supplementation enhances anti-viral innate responses: To date, all loss-of function mutations in ADA2 that cause PAN/DADA2 vasculitis either reduce protein levels of ADA2 or dramatically impair enzymatic activity of ADA2, such that loss of enzymatic activity in serum is the defining characteristic of the disease. Loss of ADA2 activity in serum alters catabolism of Ado/dAdo released into circulation from cells and tissues, or produced by dephosphorylation of extracellular ATP/dATP. Ado and dAdo are profoundly immuno-modulatory nucleosides, exerting many effects on cells. Vascular endothelial cells metabolize Ado/dAdo in blood, yet excessive or dysregulated Ado/dAdo signaling has not been examined in endothelium. To examine the effects of Ado on vascular endothelial cells, HUVEC were supplemented with extracellular Ado 24 h prior to treatment with DNA or infection with hCMV. Analysis of IRF3 activation and IFNβ gene induction clearly showed that Ado supplementation significantly enhanced innate immune cytokine production in primary human endothelial cells, to a similar degree as ADA2 depletion (FIG. 7a). Importantly, similar results for IFNβ gene induction were observed in brain, kidney and skin endothelial cells (FIG. 7b-d). Notably, enhanced expression of IL-6 and TNF cytokines was also observed after Ado supplementation (data not shown). Together, these results provide that extracellular Ado enhances innate immune responses in endothelial cells, suggesting that catalytic activity of ADA2 is involved in dampening pro-inflammatory responses.

The inventors have shown that deficiency of ADA2 in vascular endothelial cells enhances anti-viral innate immune responses, through increased production of pro-inflammatory cytokines such as IFNβ, CCL5, IL-6 and TNF.

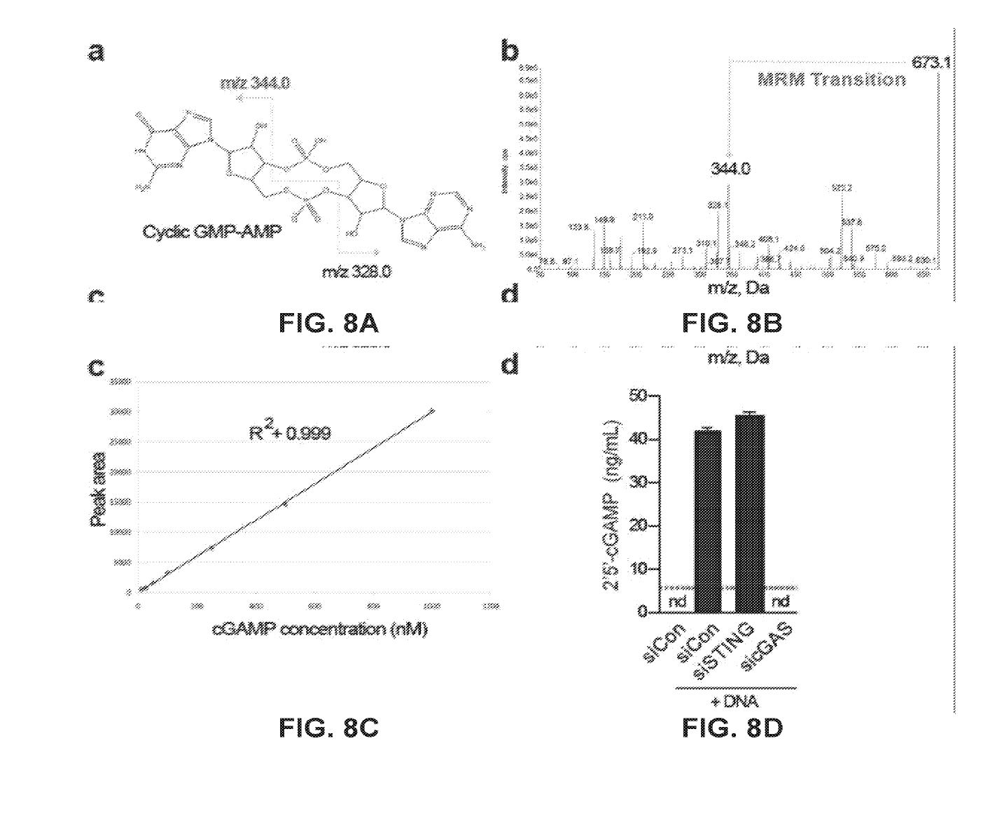

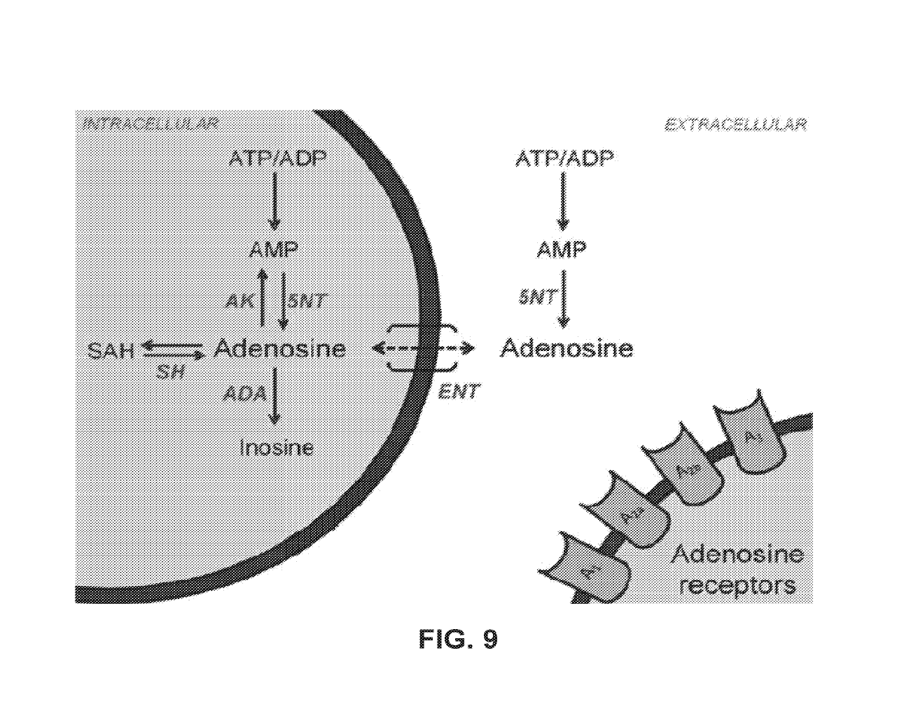

ADA2 functions to dampen cell-intrinsic innate immune responses through the STING axis (FIG. 4). To determine the precise mechanism by which ADA2 interfaces with the STING pathway (FIG. 1), a set of optimized read-out assays were used to analyze ADA2-deficient cells after challenge with DNA or infection with hCMV. Notably, each of these “check-point” assays, which quantify the major steps of the known STING cascade, were optimized. i) DNA uptake was measured by calculating the fluorescence intensity of a Cy5-labeled DNA substrate using flow cytometry, or by PCR amplification of genomic hCMV sequences. ii) cGAS activity was measured by quantifying cellular cGAMP levels using liquid chromatography-coupled tandem mass spectrometry (LC-MS/MS) (FIG. 8). Briefly, multiple reaction monitoring (MRM) to measure the most abundant ionization fragment of 2′-5′ cGAMP, the specific isoform produced by mammalian cells (m/z 344, FIGS. 8a-c), was used to detect cGAMP in the low nM range in cells (cutoff=5 ng/mL). Notably, cGAS siRNAs abolished cGAMP production (FIG. 8d). iii) STING activation was measured by quantifying the ratio of monomeric to dimerized STING by western blot. iv) TBK1 activation was measured using quantitative in situ immunofluorescence using TBK1 phospho-specific antibody. v) IRF3 nuclear translocation was quantified by in situ immunofluorescence (3). vi) DNA binding of IRF3 to target promoters in the nucleus was quantified by EMSA (24) and CHiP assay. vii) IRF3 association with the transcriptional co-activator CBP is measured by co-immunoprecipitation.

Specialized endothelial activation/effector functions in ADA2-deficient endothelial cells were assessed. For studies of endothelial activation, poly dA:dT DNA treatment or hCMV infection were used to activate STING. In addition to vehicle controls, treatment with thrombin, VEGF and LPS were used as endothelial cell activators, and BAPTA-AM as an inhibitor. The following was measured in control and ADA2-deficient endothelial cells: i) Permeability of the endothelial barrier by monitoring movement of CFSE-labeled monocytes and neutrophils across confluent endothelial monolayers. ii) Monocyte and neutrophil adhesion to endothelial monolayers is quantified by measuring CFSE. iii) Production of vascular ROS is measured in cell supernatants using the chemiluminescent probe L-012. iv) Up-regulation of immune cell adhesion molecules is monitored by flow cytometry for intracellular and surface VCAM, ICAM1 and E-selectin. v) Monocyte and neutrophil activation are assessed by induction of pro-inflammatory cytokine gene expression (e.g. IFNα/β, CCL5, MCP1, IL6, IL8 and TNF) using qRT-PCR and flow cytometry for surface activation markers. vi) Endothelial proliferation is quantified by uptake of BrdU by in situ immunofluorescence and quantitative fluorescence microscopy. vii) Apoptosis is measured by Annexin V surface expression by flow cytometry.

Circulating ADA2 is a polarization factor for anti-inflammatory M2 macrophages and an enzyme that catabolizes extracellular Ado or dAdo, biologically active purines that exert profound physiological effects on cells. The date herein shows that extracellular Ado supplementation enhances anti-viral innate immune responses in endothelial cells (FIG. 7), and the ADA2/Ado/dAdo axis plays an important role dampening pro-inflammatory responses.

ADA2/Ado axis drives enhanced innate immune pro-inflammatory responses to virus infection in vascular endothelial cells (FIGS. 4 and 7). In the absence of ADA2 enzymatic activity, there are two distinct fates for extracellular Ado directly released into the blood by cells and tissues, or produced in circulation via catabolism of secreted ATP, ADP and AMP (FIG. 9) First, Ado can act as an extracellular signaling molecule by binding to G-protein coupled purinergenic receptors (FIG. 9, Adenosine receptors A1, A2A, A2B and A3) on the surface of many different cell types, including endothelial cells, smooth muscle cells and immune cells (e.g. neutrophils, monocytes, macrophages, mast cells, dendritic cells). AdoR signaling produces a complex array of physiological outcomes, including suppressive effects on cells of the immune system. Second, Ado can be transported into red blood cells or vascular endothelial cells via plasma membrane ENT transporters (FIG. 9, ENT=ENT1, ENT2 and ENT4), where it can be rephosphorylated to AMP by adenosine kinase (i.e. purine salvage pathway), deaminated by ADA1 to produce inosine, hypoxanthine, xanthine and uric acid (i.e. excretory pathway), or converted into S-adenosyl-L-homocysteine (SAH) and shuttled into the S-adenosyl-Lmethionine (SAM) methylation pathway (i.e. transmethylation pathway) (FIG. 9).

To examine Ado/AdoR signaling, control and Ado-treated or STING, IRF3 and ADA2 knock-out endothelial cells were treated with 5′-N-Ethylcarboxamidoadenosine (NECA), a non-hydrolyzable analogue of Ado that acts as a potent AdoR agonist through AdoR isoforms A1 and A2A/B. Without being limited to any particular theory, NECA did not enhance IRF3 and IFNβ responses to DNA stimulation and hCMV infection. Single and double siRNA knockdowns of AdoR were performed in endothelial cells, which express relatively high levels of the A2A/B isoforms. Without being limited to any particular theory, if signaling through AdoR drive innate responses in endothelial cells, their depletion impairs enhanced IRF3 and IFNβ responses in ADA2-deficient cells, and have suppressive effects in control cells. Finally, the ability of AdoR antagonists, XAC (non-selective), ZM241385 (A2A-selective), enprofylline and IPDX (A2B-selective) and CPX (A1-selective, as a negative control) were examined to dampen enhanced IRF3 and IFNβ responses in ADA2-deficient cells. Without being limited to any particular theory, AdoR signaling is involved in regulating innate responses in endothelial cells, and Ado/AdoR antagonists can be applied to dampen pro-inflammatory responses in ADA2-deficient endothelial cells.

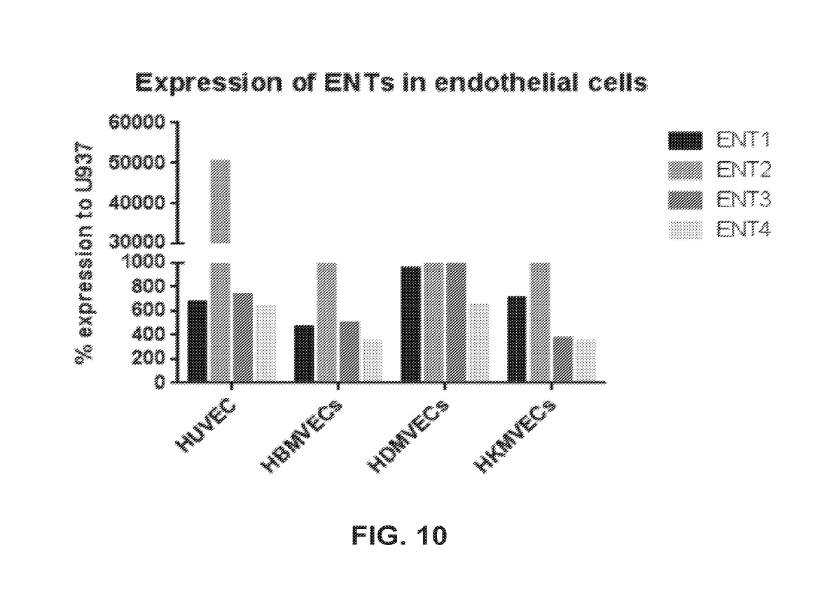

To examine the role of purine uptake and metabolism, the inventors supplemented cells with extracellular dAdo (i.e. 2′-deoxyadenosine). Like Ado, dAdo is produced in circulation from dATP and is taken up and metabolized by endothelial cells through the excretory, purine salvage or transmethylation pathways. Notably, dAdo does not signal through AdoR. Without being limited to any particular theory, the effects of ADA2 deficiency involve dysregulation of Ado and dAdo show that supplementation of extracellular dAdo significantly enhances IRF3 and IFNβ responses to DNA and hCMV, as shown for Ado (FIG. 7). In parallel, the inventors performed single and combinatorial siRNA knockdowns of the plasma membrane-localized ENT transporters ENT1, ENT2 and ENT4 in endothelial cells, which express relatively ENT expression (FIG. 10). Without being bound to any particular theory, purine uptake through ENT drives innate responses in endothelial cells, and their depletion reduces enhanced IRF3 and IFNβ responses to baseline levels or below in ADA2-deficient cells and Ado/dAdo-treated cells, and also in control cells. Finally, the inventors examined the ability of a class of drugs called Ado re-uptake inhibitors (AdoRI, which inhibit ENT transporters)—dipyridamole, dilazep hydrochloride and nitrobenzylthioinosine (NMBP)—to antagonize IRF3 and IFNβ responses to DNA and hCMV in ADA2-deficient cells and Ado/dAdo-treated cells to determine whether Ado/dAdo uptake regulates innate responses in endothelial cells and if AdoRI can be used to dampen pro-inflammatory responses.

The inventors determined the relative contribution of 1) extracellular Ado signaling through purinergenic AdoR receptors, and 2) extracellular Ado/dAdo uptake through ENT transporters in driving enhanced innate immune responses in endothelial cells. It was also examined whether well-established pharmacological modulators of Ado and dAdo signaling can be used to dampen inflammatory responses, which is directly applicable for pre-clinical studies of vasculitis (Aim 3).

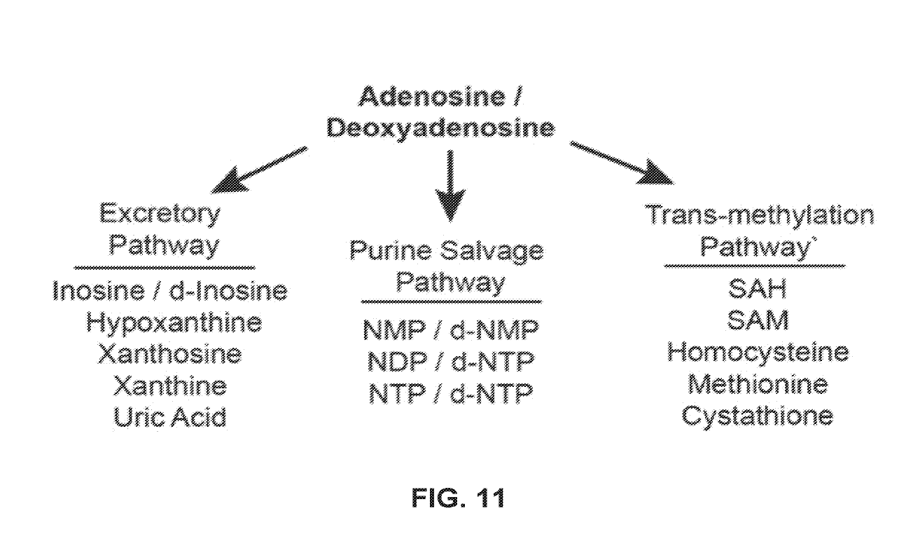

Ado and dAdo are biologically active purines that elicit profound effects on cells, and so altered Ado/dAdo catabolism/deamination is expected to deregulate cell physiology. Accordingly, disruption of ADA1 in humans causes accumulation in Ado/dAdo in cells, perturbing cellular homeostasis by disrupting nucleotide/deoxynucleotide pools and cellular transmethylation reactions. Although expression of ADA2 is more limited than ADA1, loss of ADA2 catalytic activity directly affects serum Ado/dAdo levels, which impacts many different types of cells expressing AdoR or ENT. Since vascular endothelial cells are known to rapidly clear extracellular Ado/dAdo from circulation via ENT transporters, the inventors have found that ADA2 disruption produces profound metabolic changes in endothelial cells by impacting at least three different intracellular pathways: (1) excretory pathway, (2) purine salvage pathway, and (3) transmethylation pathway (FIGS. 9 and 11), which are all influenced by Ado and dAdo via allosteric regulation or feedback inhibition.

To explore metabolic changes in ADA2-deficient cells, the inventors profiled cell lysates and culture supernatants prepared from control and ADA2-deficient endothelial cells, either left unstimulated or stimulated with DNA or hCMV infection. In parallel, the inventors analyzed metabolic changes in cells supplemented with extracellular Ado or dAdo, untreated or treated with the ENT inhibitors described above. Metabolomic profiling studies were performed using state-of-the-art liquid chromatography coupled to high sensitivity mass spectrometry (LCMS/MS), as previously described. Briefly, control or ADA2-deficient cells grown to confluent monolayers in standard EBM2 or EBM2-MV media supplemented with growth factors (Lonza) were left unstimulated, stimulated with DNA or infected with hCMV. LC-MS/MS analysis was performed. Cells or spent medium (100 ul) samples underwent metabolite extraction using 1 mL of pre-chilled 50:50 methanol:water followed by collection, vortexing and repeat freeze-thaw cycles. Samples were centrifuged and supernatant-containing extracted metabolites were collected for analysis. Samples were dried in vacuo using a vacuum concentrator, resuspended in 40 uL of 80:20 methanol:water, and transferred to LCMS vials at 4 C for analysis. LC-MS/MS based metabolite measures were performed using an Agilent 1290 UPLC coupled to an Agilent 6495 iFunnel Triple Quadrupole mass spectrometer, operating in high sensitivity sMRM mode. LC separation was performed using a Millipore (Sequant) Zic-pHILIC 2.1×150 mm 5 mm column maintained at 25° C., as described, with subsequent sMRM mass analysis. Importantly, MRM schedules (parent and daughter masses) were pre-optimized using MS grade pure standards for all target metabolites (FIG. 11), with confirmed measure in endothelial cell samples. Data was analyzed using MultiQuant software (Agilent). Significant changes were defined as greater than 1-fold change in the average log 2 transformed value of counts for each metabolite measured in ADA2-deficient cells or Ado/dAdo-treated cells compared to controls.

Validation of metabolomic profiling was done using functional genomics (RNAi and CRISPR). For each target metabolite significantly altered in ADA2-deficient cells, the regulatory enzymes in the pathway were identified using the KEGG database, apply RNAi, CRISPR or cDNA techniques to deplete or over-express each enzyme in endothelial cells, and assign a functional role. The cellular supplementation of each metabolite was also examined.

Development of vasculitis in TREX1-deficient mice: Both the STING and Ado/dAdo signaling pathways are highly conserved in mouse. In mice, a single Ada isoform is both intracellular and secreted, whose deletion results in perinatal lethality due to severe liver degeneration. Although Ada-deficient fetuses are rescued by transient re-introduction of Ada during development, the mice display dysregulated Ado/dAdo levels in cells and serum, along with lymphopenia (SCID) as well as lung inflammation. Rather than dissect the complex and contradictory phenotypes of Ada-deficient mice, the inventors examined TREX1-deficient mice obtained from Taconic Biosciences. These mice recapitulated many multi-systemic, inflammatory features of human AGS, an IFN-I-induced autoinflammatory disease that is clinically similar to human PAN/DADA2 vasculitis. A benefit of using TREX1-deficient mice is that infection is not needed to trigger STING, as “sterile inflammation” is spontaneously induced by accumulation of endogenous DNA elements in the cytosol (e.g. retrotransposable elements).

Endothelial STING vs myeloid STING: TREX1−/− mice display inflammatory vasculitis affecting the skin, brain, heart, gastrointestinal tract and skeletal muscle, and succumb at a median age of 10 weeks, primarily due to circulatory failure. In pathogen-free conditions, disease is entirely dependent on STING-IFN-I signaling (FIG. 1), as genetic ablation of STING, IRF3 or IFNAR (i.e. type I IFN receptor) in TREX1−/− mice completely prevents disease. Tissue-restricted expression of IFN-I mediates disease pathogenesis in TREX1−/− mice: initial induction of IFN-I in TREX1−/− mice originates from non-hematopoietic cells, such as the endocardial cells of the heart, which are endothelial in origin. Mixed bone marrow chimaeras showed that TREX−/− deficiency in non-hematopoietic cells was sufficient to drive disease even in the presence of a wild type hematopoietic system and that TREX1 deficiency in hematopoietic cells, specifically lymphocytes, was not required to initiate the auto-immune response. Without being bound to any particular theory, cell-intrinsic STING-IFN-I signaling in vascular endothelial cells is an important driver of IFN-I-driven vasculitis syndromes such as AGS. This is further supported by 1) high expression of STING in vascular endothelial cells; 2) clinical manifestations of vasculopathy in patients harboring gain-of-function mutations in STING; 3) robust STING-dependent endothelial responses to DNA or DNA virus infection; 4) Ado/dAdo-driven enhancement of STING responses in endothelial cells.

To study endothelial IFN-I responses and vasculitis in TREX1−/− mice, the inventors conditionally-targeted STINGflox/flox mice. STINGflox/flox mice were crossed with two different Cre-driver strains obtained from Jackson Laboratories, Tie2-Cre (endothelial cells) and Cd11b-Cre (myeloid cells). These strains and STINGgt/gt mice (whole body STING knock-out mice, generated by ENU mutagenesis were bred with TREX1−/− mice. Survival: Median survival of DKO mice (STINGgt/gt/TREX1−/−, STINGflox/flox-Tie2Cre/TREX1−/− and STINGflox/flox-Cd11bCre/TREX1−/−) was compared to TREX1−/− mice and control floxed littermates. TREX1−/− mice lacking STING expression in endothelium were significantly protected from auto-immune lethality, to a similar or even greater degree as myeloid STING-deficient mice. STINGgt/gt/TREX1−/− were completely protected. Histology: Histological analysis of paraffin-embedded tissue sections from heart, skeletal muscle, skin and brain from control and DKO mice was performed at 2-week intervals starting at 4 weeks of age, which signals the onset of disease in TREX1−/− mice, to monitor inflammatory infiltration and histological score. Endothelial activation/inflammation markers were assessed in CD31-expressing cells by immunohistochemical staining for ICAM1, VCAM1 and E-selectin. IFNβ were also probed in CD31 (endothelial) or CD45 (hematopoietic) positive cells. Cytokine levels: Innate cytokine levels were analyzed in serum of control and DKO mice over time. IFN-I was measured from serial bleeds via commercial ELISA (IFNβ, IFNα4 and polyclonal-IFNα) and bioassay (ISRE-luciferase L929 cells). Other STING-related cytokines/chemokines were also measured in organs and sera (e.g. IL-12, IL-18, IL-1β, MIP-1α, CCL5, CXCL1, CXCL10). As many locally produced cytokines do not efficiently access circulation, mRNA analysis by qRT-PCR and/or ELISA from organ homogenates was performed. Inflammatory cytokine levels were significantly reduced in endothelial STING-deficient cells and myeloid STING-deficient cells. Significant delay and amelioration of inflammatory vasculopathy and decreases in endothelial activation and inflammation markers was also observed.

Pharmacological manipulation of Ado/dAdo signaling and vasculitis: The data herein shows that ADA2/Ado/dAdo signaling regulates IFN-I responses in vascular endothelial cells (FIGS. 4, 5 and 7). Ado/dAdo are constitutively produced and released in circulation under resting and inflammatory conditions, and Ado/dAdo signaling regulates pro-inflammatory responses in vascular endothelial cells during initiation and progression of auto-immune vasculitis. Pharmacological modulators of Ado/dAdo signaling and metabolism were used to perform pre-clinical studies examining progression of vasculitis in vivo. Notably, all of these modulators are well tolerated and well characterized for use in mice and humans. Antagonists of AdoR signaling: To determine if modulators of Ado:AdoR signaling (ZM241385, IPDX) affect vasculitis, TREX1−/− mice were treated with a drug course beginning at 4 weeks of age, as previously described. Progression of disease was analyzed by survival, histology, and expression of pro-inflammatory cytokines. Inhibitors of Ado/dAdo:ENT signaling: To determine if antagonists of Ado/dAdo uptake and metabolism (dipyridamole, dilazep hydrochloride) affect development of vasculitis, TREX1−/− mice were treated with these agents at 4 weeks of age, as previously described, and analyzed as detailed above.

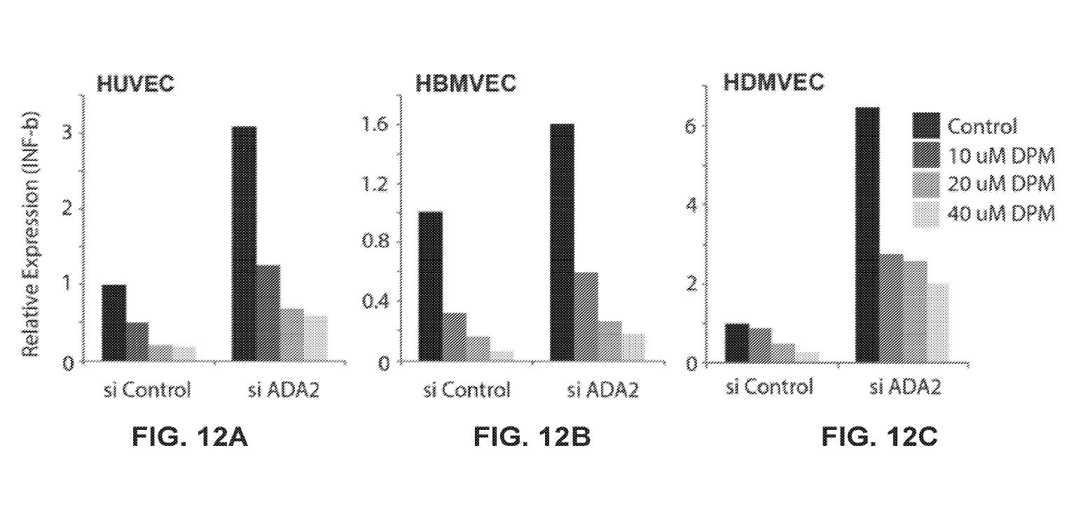

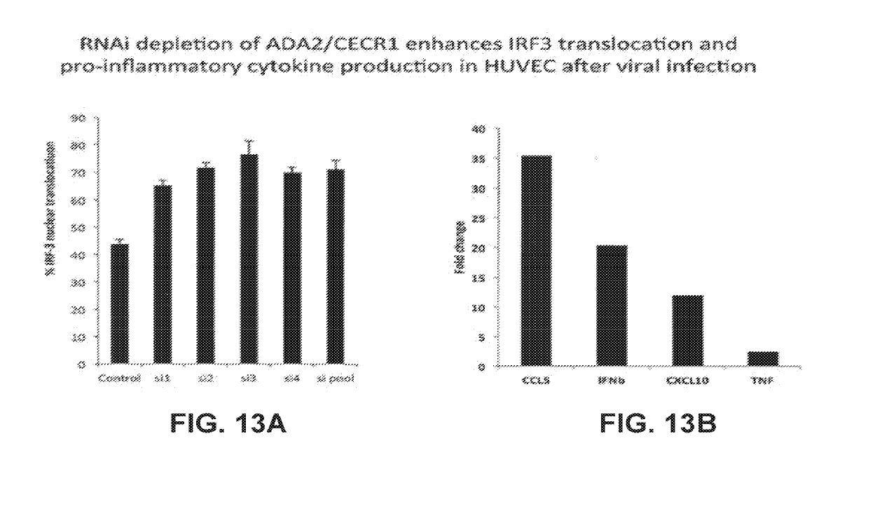

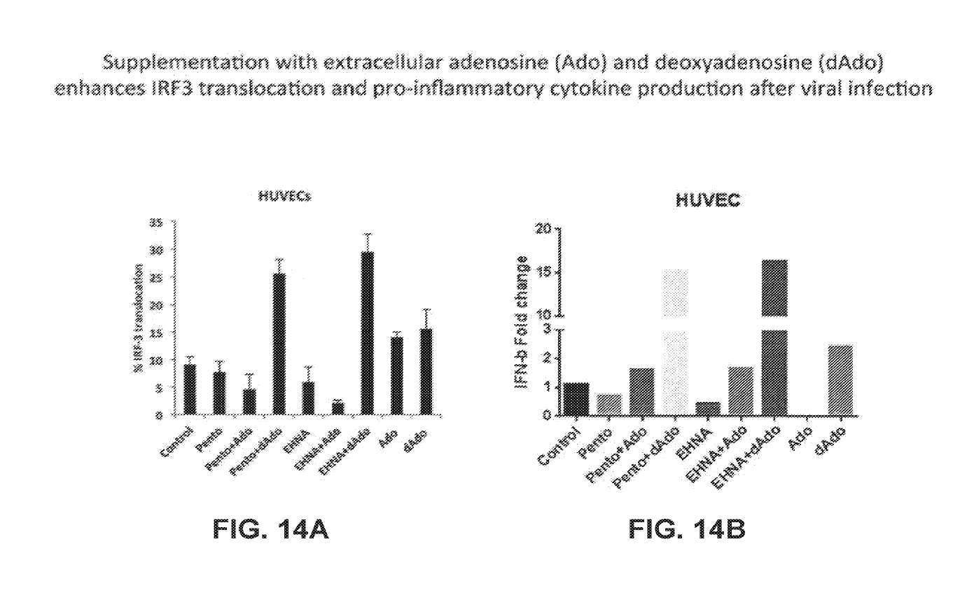

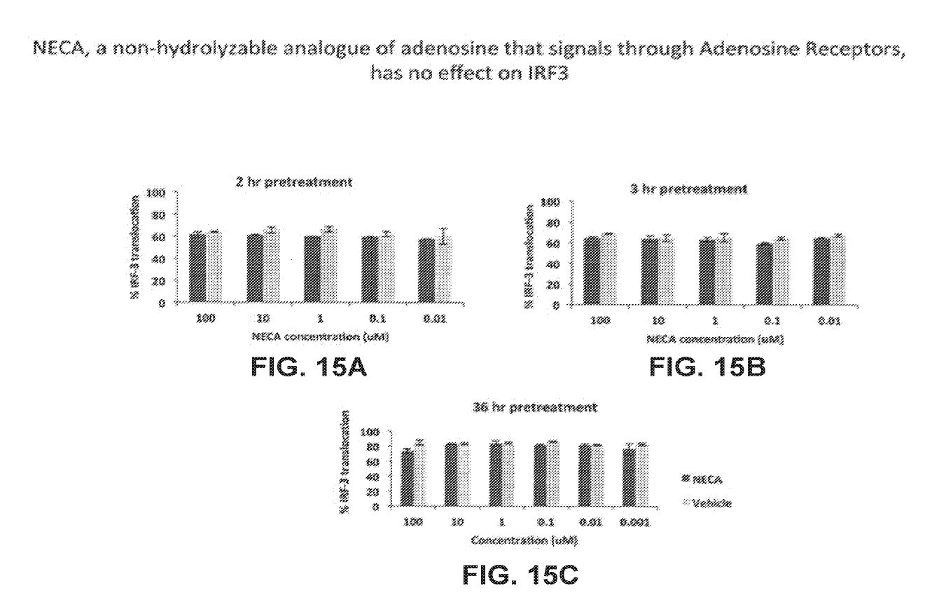

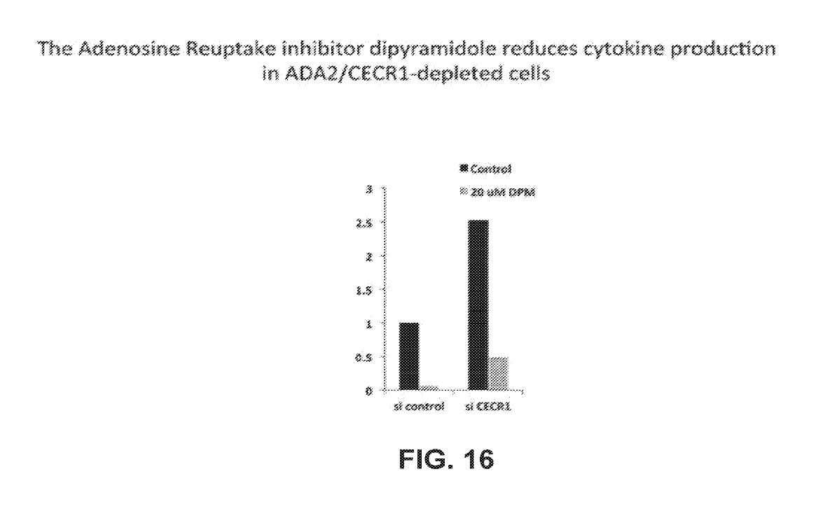

Endothelial STING-IFN-I signaling was examined in the pathobiology of vasculitis. Based on data provided herein, STING expression in endothelial cells is essential for initiation, progression and severity of cytokine production, vascular inflammation and immune cell infiltration in affected tissues, to a similar or even greater degree as myeloid STING. Pharmacological inhibition of AdoR signaling or AdoRI uptake significantly alters development of vasculitis in vivo. Importantly, pre-treatment of endothelial cells with dipyridamole significantly reduced IFNβ cytokine induction by virus infection in both control and ADA2-depleted cells (FIG. 12), confirming that ADA2/Ado/dAdo signaling/uptake/metabolism is an important regulator of cell-intrinsic innate responses in endothelial cells. Dipyridamole, and other drugs that modulate Ado/dAdo signaling, alleviates the pathogenesis of IFN-I-induced vascular inflammation observed in TREX1−/− mice.

### Example 4

Through functional screening in primary human cells, the inventors identified Adenosine Deaminase 2 (ADA2) as an inhibitor of the innate immune response to virus infection via the STimulator of Interferon Genes (STING) pathway. Deficiency of ADA2 (DADA2) is a multi-organ inflammatory disease of recurrent fevers, skin rashes and lacunar strokes^{1,2}. Here the inventors have shown that purine nucleosides prime expression of innate immune response genes. Low ADA2 activity or accumulation of extracellular nucleosides drives cellular uptake and catabolism of Ado/dAdo, yielding intracellular immuno-metabolites that drive over-expression of STING, type I Interferon (IFNβ) and many pro-inflammatory genes via inhibition of S-adenosyl-methionine (SAM) metabolism and SAM-dependent DNA methylation. The inventors discovered a metabolic-epigenetic rheostat for highly inducible innate immune genes, implicating the ADA2/SAM axis in a wide variety of inflammatory conditions where innate immunity, nucleoside accumulation and DNA hypomethylation play an important role in inflammation, including infection, auto-immunity, and cancer.

### Example 5

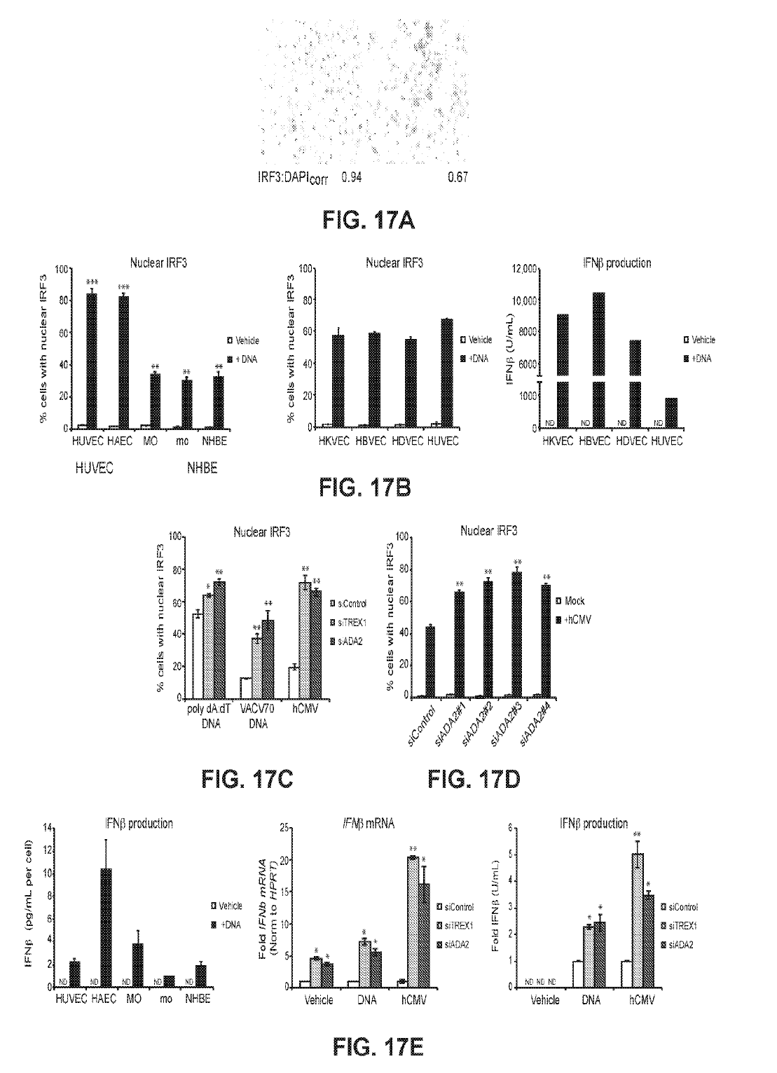

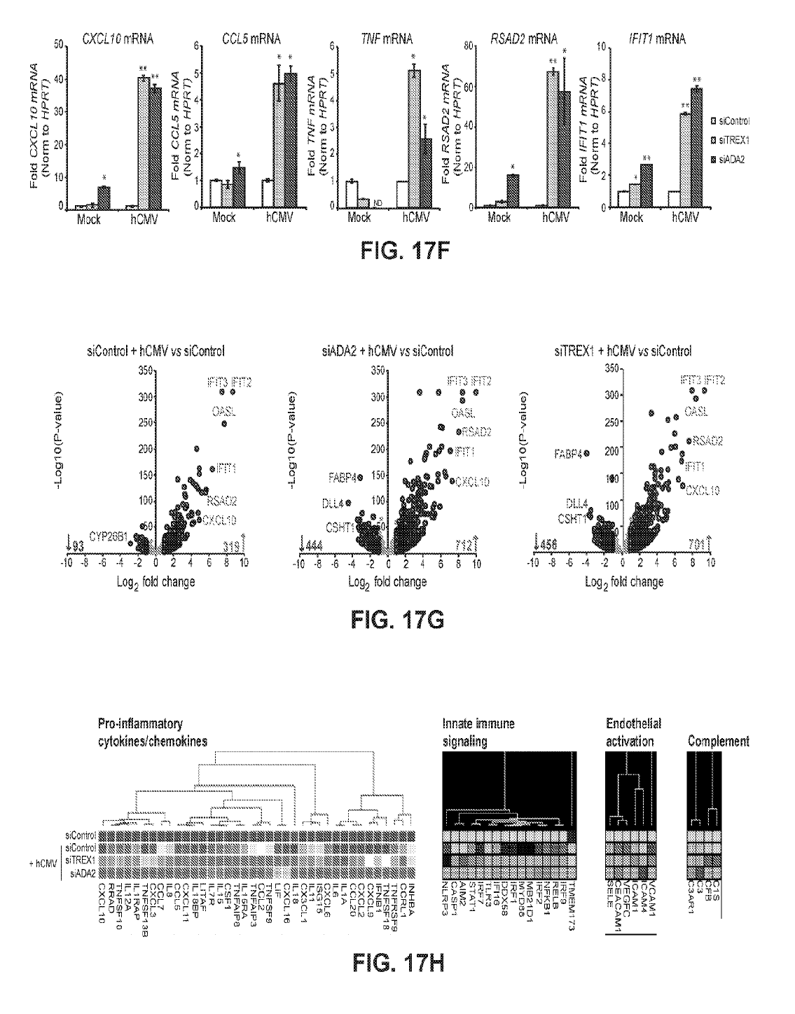

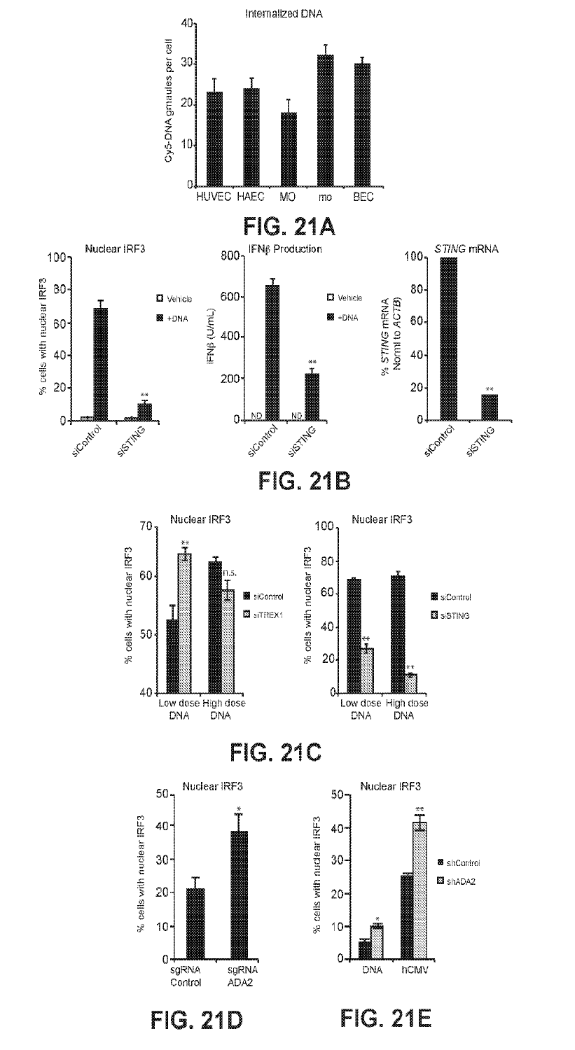

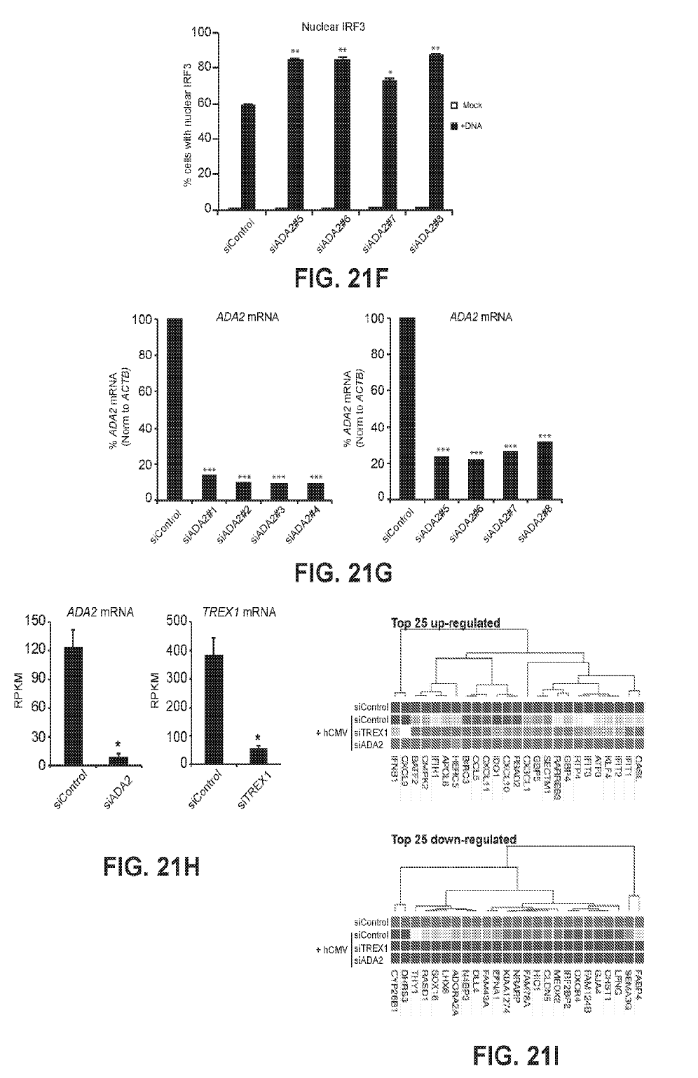

Cell-intrinsic innate immunity constitutes the early, essential and ubiquitous response of pathogen-susceptible cells to viruses, bacteria, fungi and tumors⁴. An integral part of this universal defensive response involves induction of the immune-modulatory cytokine IFNβ by infected or exposed cells^{5,6}. To monitor IFNβ signaling the inventors used an automated in situ immunofluorescence to quantify inducible nuclear translocation of the transcription factor Interferon Regulatory factor 3 (IRF3)^{7,8}, a crucial component of the IFNβ transcriptional enhanceosome⁹. Upon delivery of immuno-stimulatory dsDNA to the cytosol via transfection, which induces multi-site phosphorylation of IRF3 by STING and the anti-viral kinase Tank Binding Protein 1 (TBK1), the percentage of cells harboring nuclear IRF3 was significantly higher in endothelial-lineage cells (HUVEC, HAEC) compared to macrophages (MO), monocytes (mo) and normal human bronchial epithelial cells (NHBE) (FIG. 17a), although dsDNA internalization was equally efficient across cell types (FIG. 21a). In HUVEC, the degree of IRF3 co-localization with nuclear DAPI was uniformly higher at the single-cell level (FIG. 17a). Robust IRF3 and IFNβ responses to dsDNA were observed in all endothelial-lineage cells tested (FIG. 17b), and abolished by depletion of STING (FIG. 21b).

### Example 6

Over-production of IFNβ drives auto-inflammatory disease^{10,11}. Gain-of-function mutations in STING drive STING-Associated Vasculopathy with onset in Infancy (SAVI)^{12,13}′ while loss-of-function mutations in TREX1, a negative regulator of STING, cause systemic inflammation in Aicardi-Goutières Syndrome (AGS)¹⁴⁻¹⁶. The inventors searched the Online Mendelian Inheritance in Man (OMIM) database for other diseases and disease-linked genes associated with clinical features of inflammatory vasculitis (Table 1). To identify new regulators of the STING-IFN-I axis, the inventors disrupted disease-linked genes in HUVEC using RNAi and CRISPR/Cas9 techniques, and demonstrated that depletion of ADA2 significantly enhanced nuclear IRF3 accumulation upon transfection of immuno-stimulatory dsDNAs or infection with human cytomegalovirus (hCMV) (FIG. 17c-d and FIG. 21c-f), a dsDNA-encoding viral pathogen that potently activates STING-IFNβ signaling. In DADA2 disease, activation of the vascular endothelium is observed in skin, brain, kidney and many other organs^{1,2}. Infection with hCMV and other dsDNA-encoding viral pathogens is a well-established trigger of disease^{17,18}, which suggests that anti-viral immune responses drive patho-inflammation. IFN—β stimulated gene signatures (ISG) are detectable in the peripheral blood mononuclear cells of DADA2 patients¹⁹. ADA2 depletion in endothelial cells resulted in enhanced IFNβ production in response to dsDNA or hCMV infection (FIG. 17e), concomitant with over-expression of many pro-inflammatory and anti-viral ISG (FIG. 17f). Whole-transcriptome RNAseq confirmed higher induction of ISG in ADA2-depleted cells, and showed that more genes were significantly altered (FIG. 17g-h and FIG. 21g-h, 412 genes in siControl, 1156 genes in siADA2, 1157 in siTREX1).

### Example 7

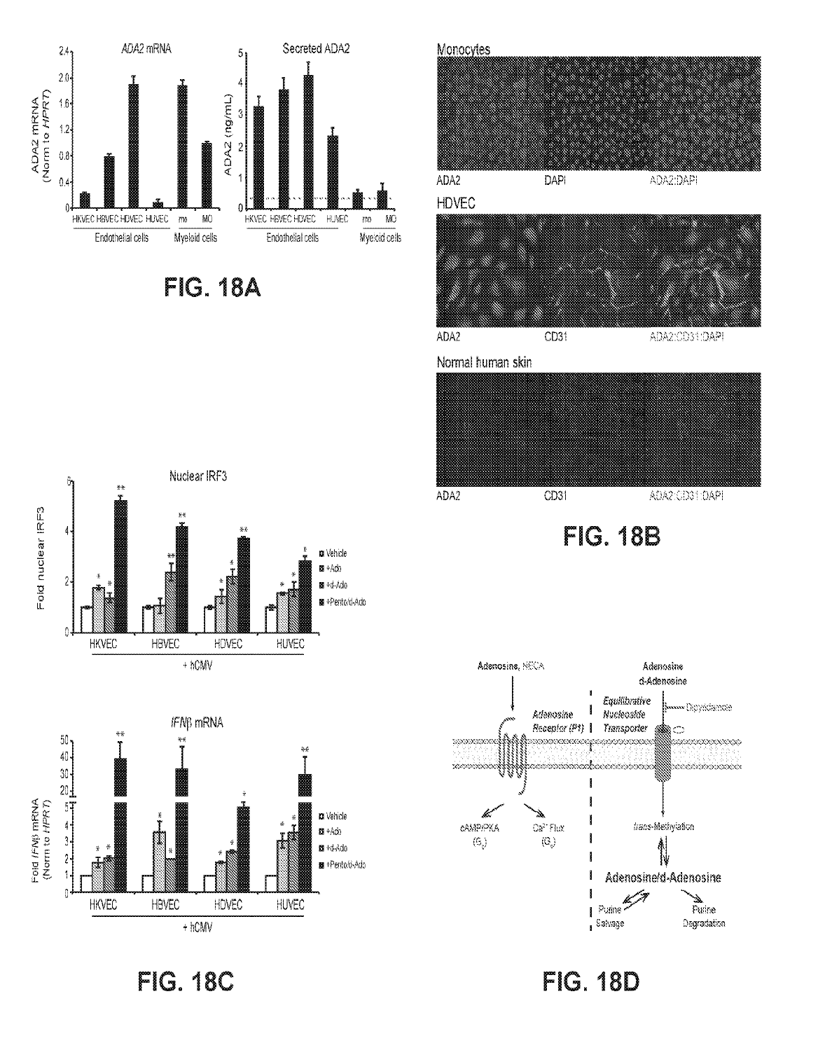

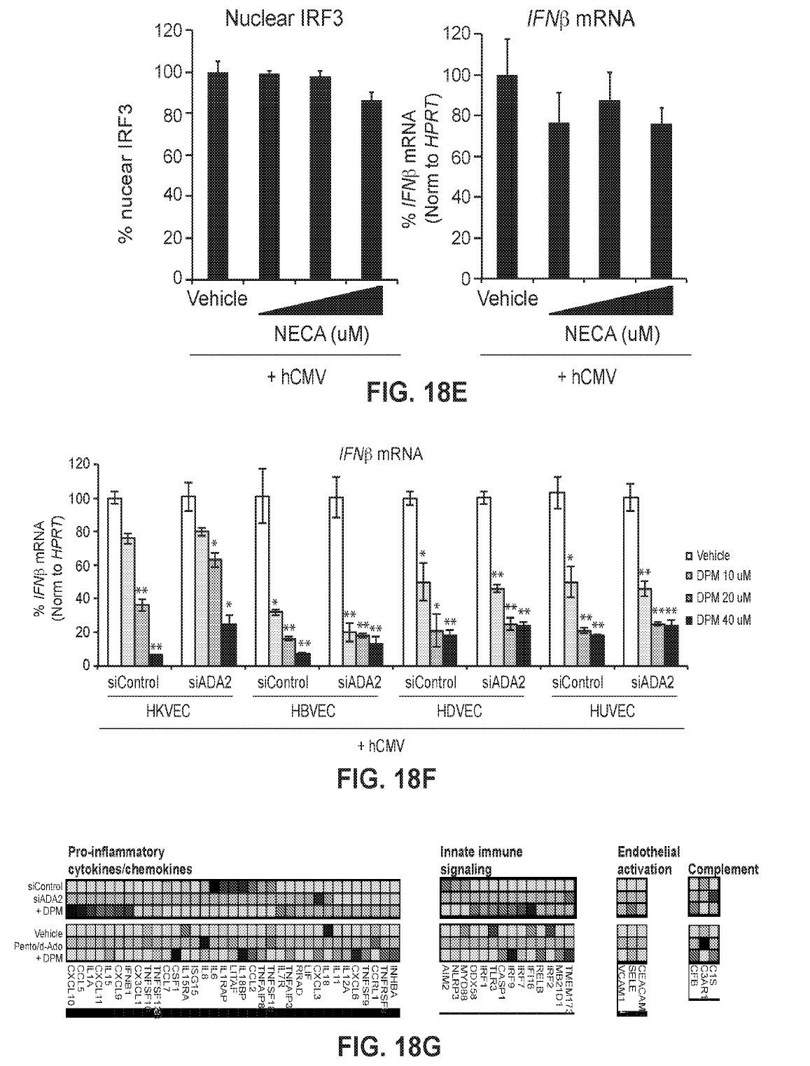

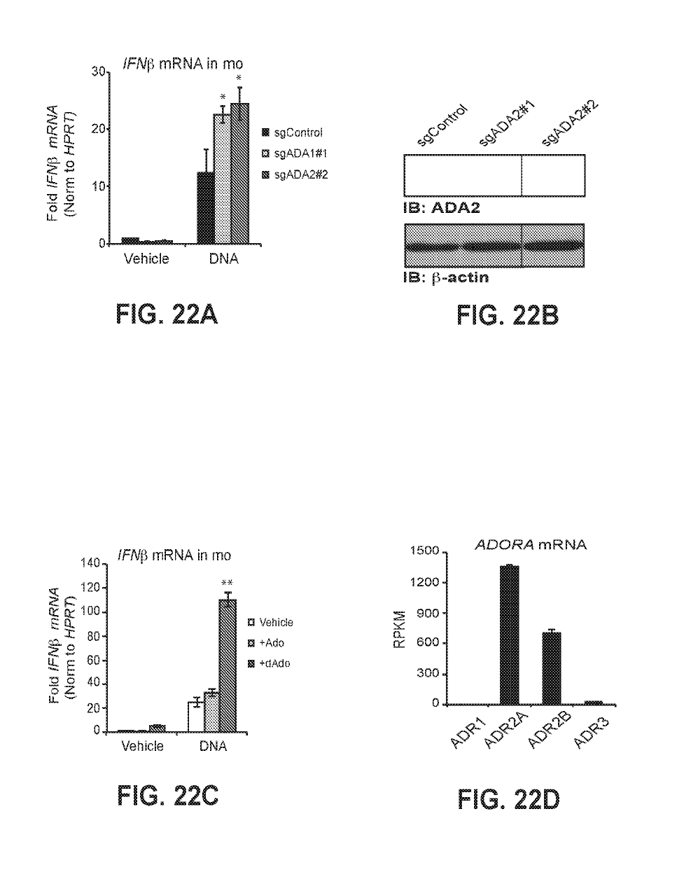

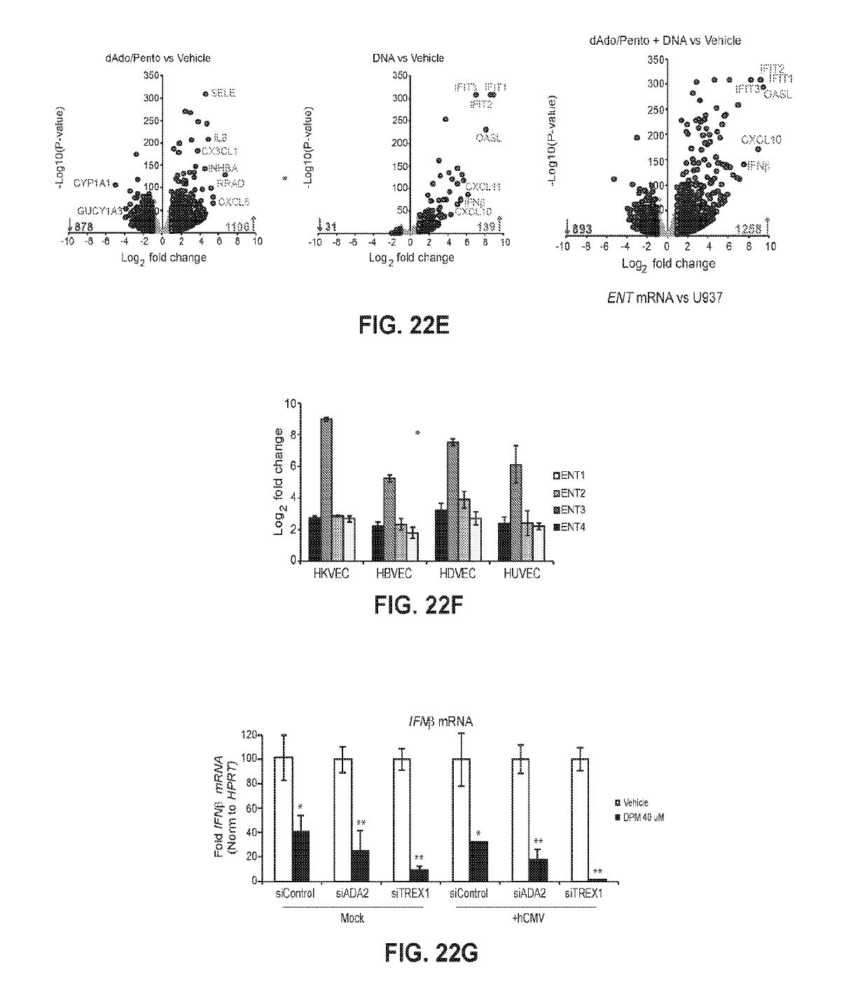

Irreversible deamination of Ado and dAdo inside and outside cells produces inosine (Ino) and deoxyinosine (dIno)³, which participate in downstream metabolic reactions. ADA1 is an intracellular enzyme that is ubiquitously expressed, while secreted ADA2 displays tissue-restricted expression^{1,2}. ADA2 is highly expressed in immune cells (FIG. 25a). The inventors detected ADA2 mRNA in monocytes and endothelial cells, and confirmed protein expression in cultured cells, supernatants and vascularized skin tissue (FIG. 18a-b). In endothelial cells, supplementation of extracellular Ado and dAdo augmented IRF3 and IFNβ responses (FIG. 18c), which was enhanced in the presence of pentostatin to inhibit ADA2. In monocytes, disruption of ADA2 and dAdo supplementation enhanced IFNβ induction (FIG. 22a-b). RNAseq of dAdo/Pento-supplemented HUVEC confirmed profound changes in many immuno-modulatory genes and ISG (FIG. 22c).

### Example 8

Purine nucleosides are biologically active molecules that exert immuno-modulatory effects during infection, injury, ischemia and tumor growth^{20,21}. Ado and dAdo are released by pathologically inflamed cells and tissues, or produced into circulation through catabolism of secreted nucleotides. In the extracellular milieu, Ado signals as a second messenger via G-protein coupled adenosine receptors (AdoR) (FIG. 18d), producing a complex array of physiological outcomes, many of which are immuno-suppressive²¹. To examine AdoR signaling, cells were supplemented with 5′-N-ethylcarboxamidoadenosine (NECA), a non-hydrolyzable analogue of Ado, to stimulate AdoR expressed by HUVEC (FIG. 22d). NECA did not enhance IRF3 or IFNβ responses (FIG. 18e), suggesting a requirement for nucleoside metabolism. In addition to extracellular catabolism by ADA2, Ado and dAdo can be taken up and metabolized inside cells via purine salvage, purine degradation and trans-methylation pathways²⁰ (FIG. 18d). Indeed, rapid uptake of circulating Ado/d-Ado by the vascular endothelium occurs within a few seconds²²⁻²⁴, consistent with the relatively high levels of Equilibrative Nucleoside Transporters (ENT) expressed by these cells (FIG. 22e). The adenosine reuptake inhibitor Dipyridamole (DPM) reduced IFNβ induction in control and ADA2-depleted cells in a dose dependent manner (FIG. 18f); DPM broadly inhibited the expression of pro-inflammatory and anti-viral genes in both ADA2-depleted and dAdo-supplemented cells (FIG. 18g), and also dampened IFNβ levels in TREX1-depleted cells (FIG. 22d), suggesting that cellular uptake and metabolism of purine nucleosides is a universal mechanism for priming innate immune response genes.

### Example 9

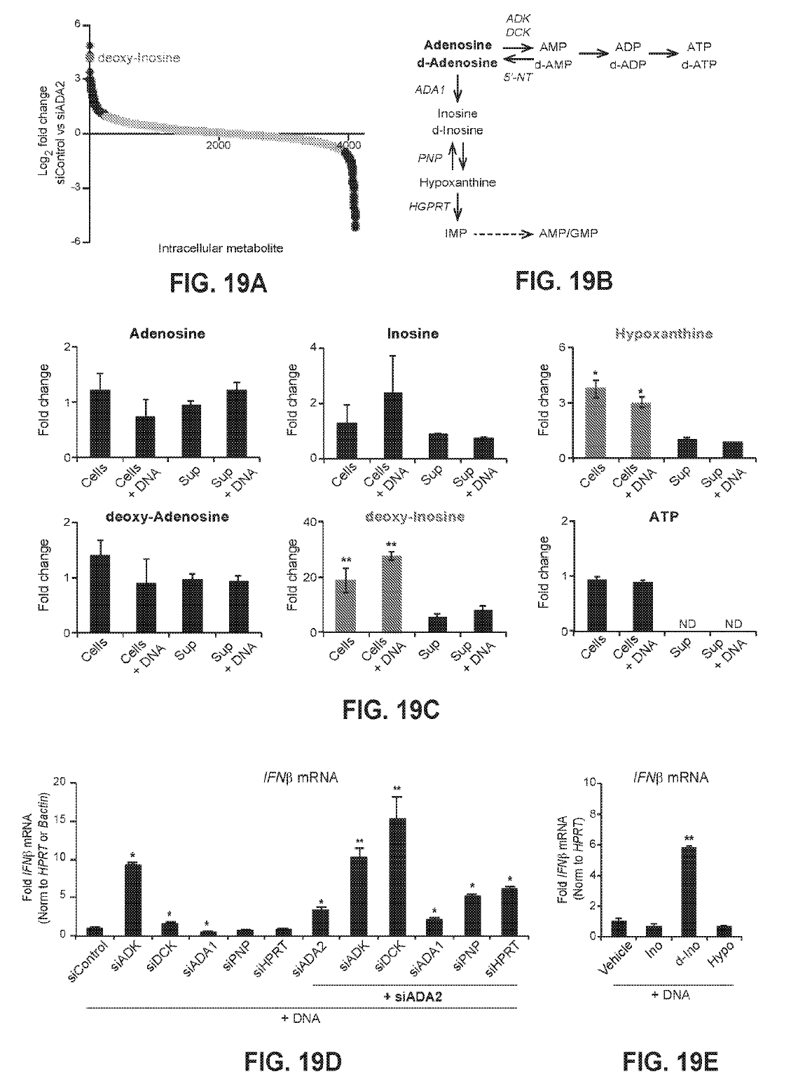

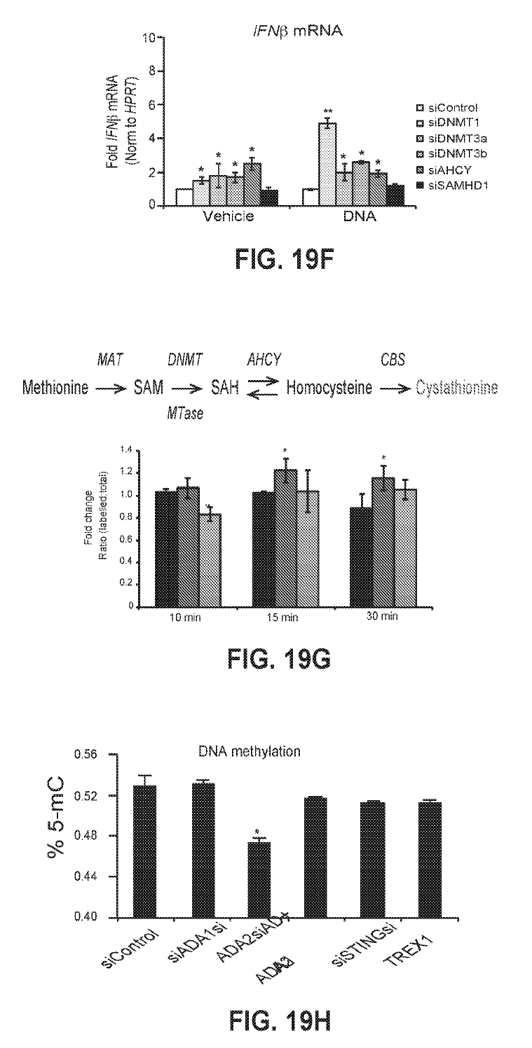

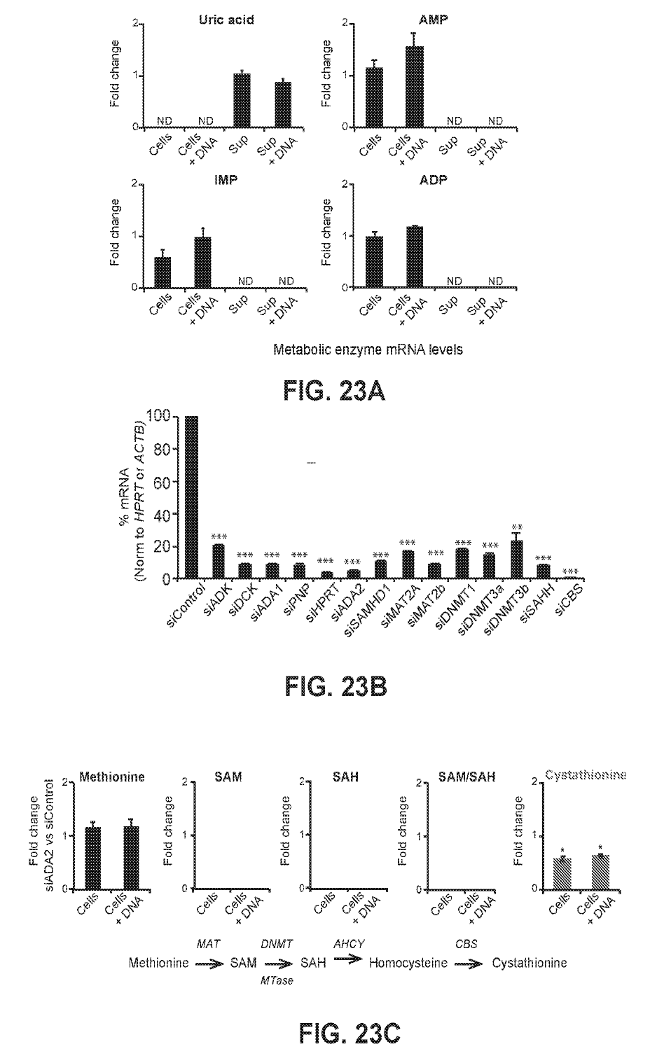

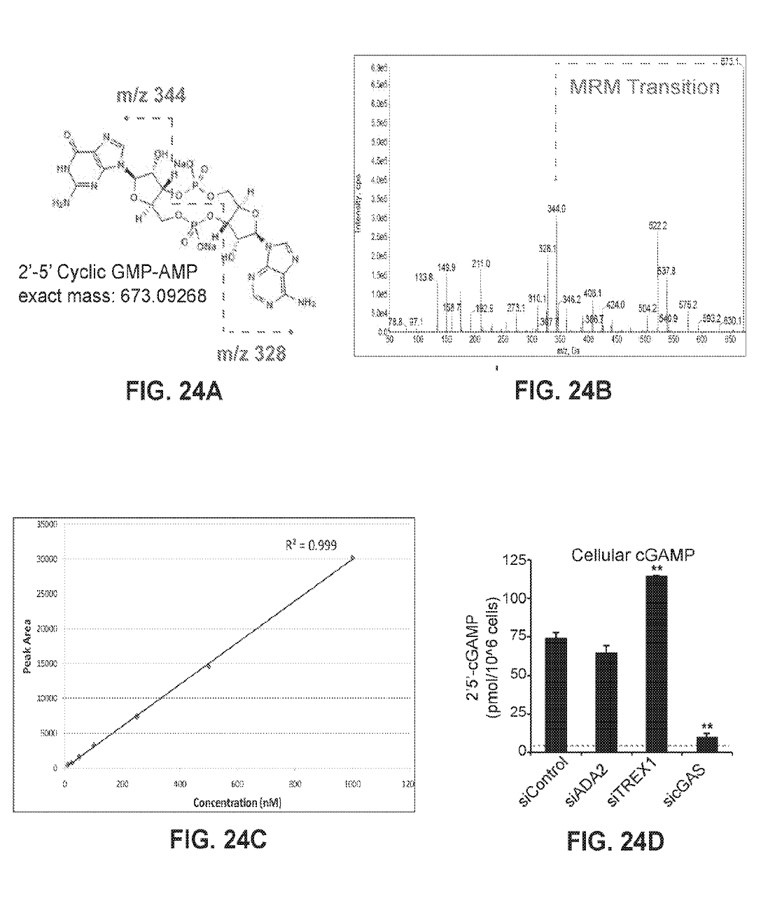

To assess metabolism in ADA2-deficient cells, the inventors simultaneously profiled thousands of small polar molecules using a large-scale, untargeted mass spectrometry-based method²⁵. Ranked evaluation of intracellular metabolites showed 19.2-fold accumulation of dIno in ADA2-depleted cells (FIG. 19a), confirming that loss of ADA2 promotes dysregulation of intracellular purine nucleoside metabolism (FIG. 19b). Hypoxanthine, the next downstream intermediate in the purine degradation pathway²⁶, was also increased by 3.7-fold (FIG. 19a). IMP, Uric acid and purine salvage metabolites ATP, ADP and AMP were not altered (FIG. 19a and FIG. 23a). Next, the inventors disrupted the enzymes controlling intracellular purine metabolism (FIG. 19b). Depletion of ADA1 did not increase IFNβ levels in both control and ADA2-depleted cells (FIG. 19d). Interestingly, ADA1 mutations account for nearly 15% of human primary immune deficiency²⁷. In contrast, disruption of ADK, DCK, PNP and HPRT not only failed to reduce IFNβ induction but significantly enhanced it in ADA2-depleted cells, emphasizing that flux through ADA1 primes IRF3/IFNβ responses. Accordingly, prolonged supplementation of cells with dIno specifically augmented IFNβ in a DPM-sensitive manner (FIG. 19e).

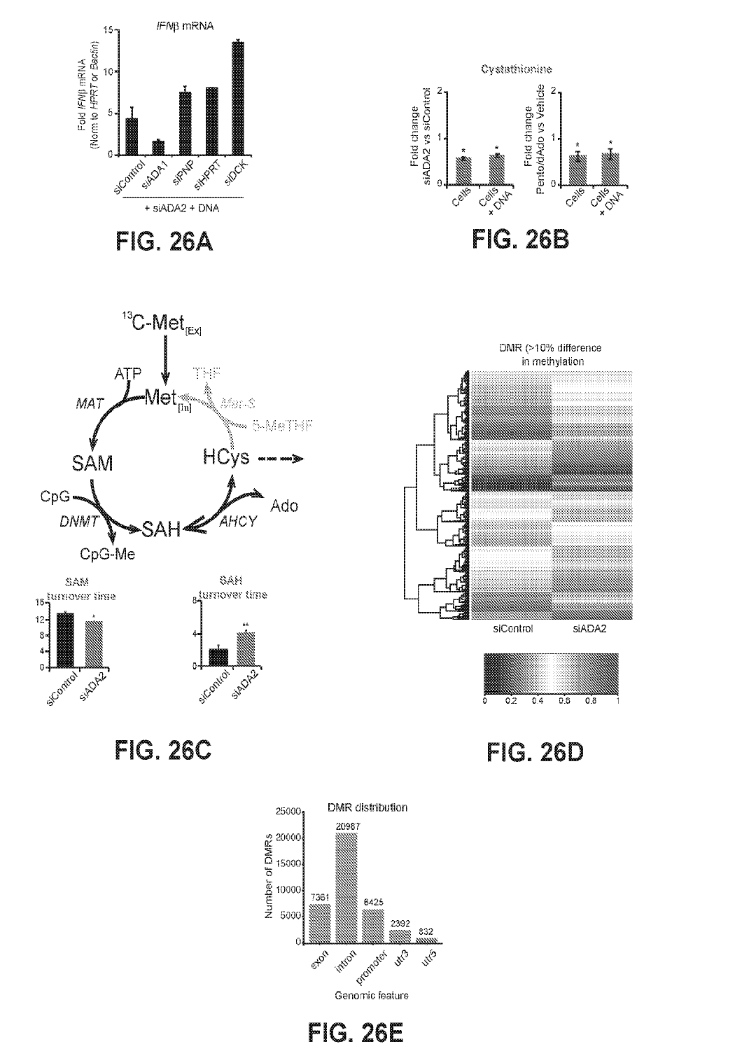

Loss of ADA1 suppressed IFNβ induction in ADA2-depleted cells, as shown in FIG. 26a. In ADA2-depleted and dAdo/pento-supplemented cells steady-state levels of L-cystathionine, a downstream metabolite of cellular trans-methylation through the S-adenosyl-methionine (SAM) cycle, were consistently reduced (FIG. 26b), suggesting potential alterations in SAM metabolism. The inventors performed kinetic metabolic flux analysis of the SAM cycle using extracellular ¹³C-labelled methionine²⁷. Labeled methionine rapidly equilibrated within intracellular metabolite pools within minutes and rapidly distributed into SAM cycle metabolites. There was little evidence of re-methylation from homocysteine back to methionine, suggesting a truncated cycle in primary cells. Kinetic labeling uncovered accelerated turnover from methionine to SAM in ADA2-depleted cells, with significantly reduced turnover from SAM to S-adenosylhomocysteine (SAH), suggesting marked impairment of the methyl transferase reaction (FIG. 26c). To examine changes in the DNA methylome the inventors performed whole genome sequencing after bisulfite treatment (WGBS), uncovering a total of 26,935 ADA2-associated Differentially Methylated Regions (DMR) (FIG. 26d) primarily distributed within gene introns, exons and promoter regions (FIG. 26e) and consisting of regions of hypomethylation (11,920) and hypermethylation (15,015). Consistent with the metabolic and WGBS observations, functional studies confirmed that perturbing cellular DNA methylation by depleting the DNA methyltransferases DNMT1, DNMT3a and DNAMT3b or the SAH hydrolase (AHCY) augmented IFNβ induction (FIG. 26f and FIG. 23b).

### Example 10

Intracellular accumulation of deoxynucleosides influences several fundamental processes, including intracellular balance of dNTPs or de novo synthesis of S-adenosyl-methionine (SAM), the donor for cellular trans-methylation reactions. Loss-of-function mutations in SAMHD1, the triphosphohydrolase that controls cellular dNTP pools, cause a clinical sub-type of AGS with features of DADA2¹⁹. However, disruption of SAMHD1 expression did not affect IFNβ levels (FIG. 19f and FIG. 23b). In contrast, depletion of the SAM pathway enzymes AHCY, DNMT1, DNMT3a and DNMT3b (FIG. 19h) significantly augmented IFNβ induction (FIG. 19f and FIG. 23c-d), suggesting that SAM metabolism and/or DNA methylation regulate innate immune expression. ISG expression was recently shown to be highly up-regulated in tumors treated with DNMT inhibitors²⁸. Regarding SAM metabolism, ADA2-depleted cells consistently showed lower levels of L-cystathionine, the by-product of a key metabolic junction that drives homocysteine into the trans-sulfuration pathway in favor of methionine/SAM re-synthesis (FIG. 19g). To examine real-time flux through the SAM pathway, cells were supplemented with labeled methionine. (FIG. 19h). Global methylation of genomic DNA was reduced in ADA2-depleted cells, and restored by additional disruption of ADA1 (FIG. 19i).

### Example 11

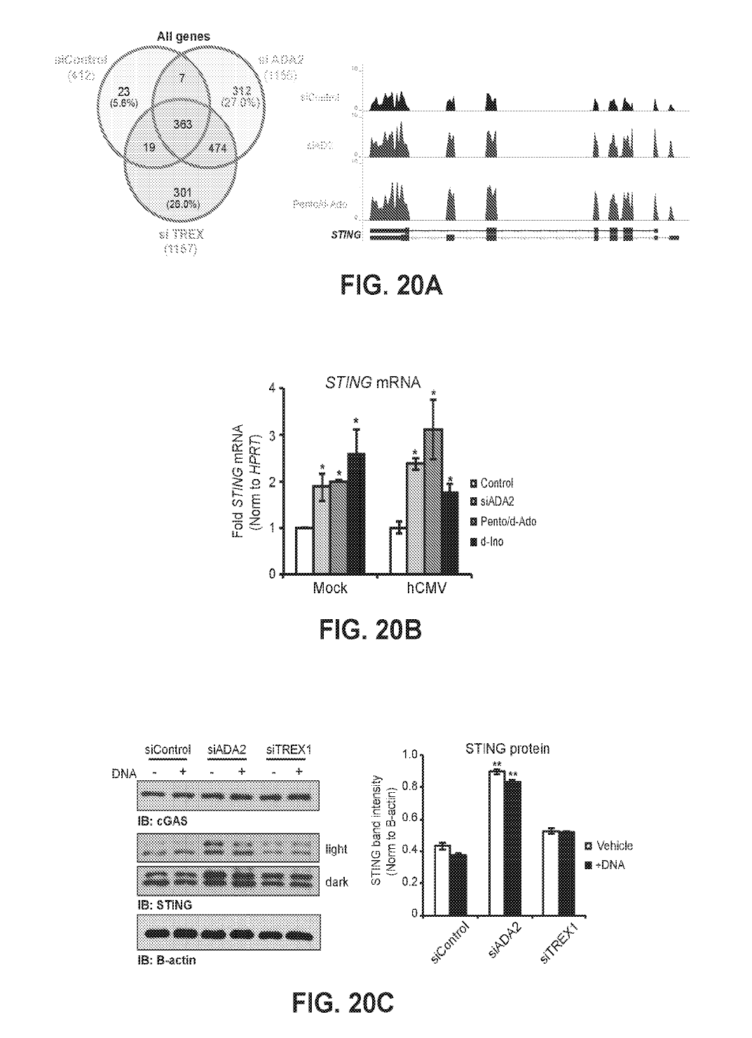

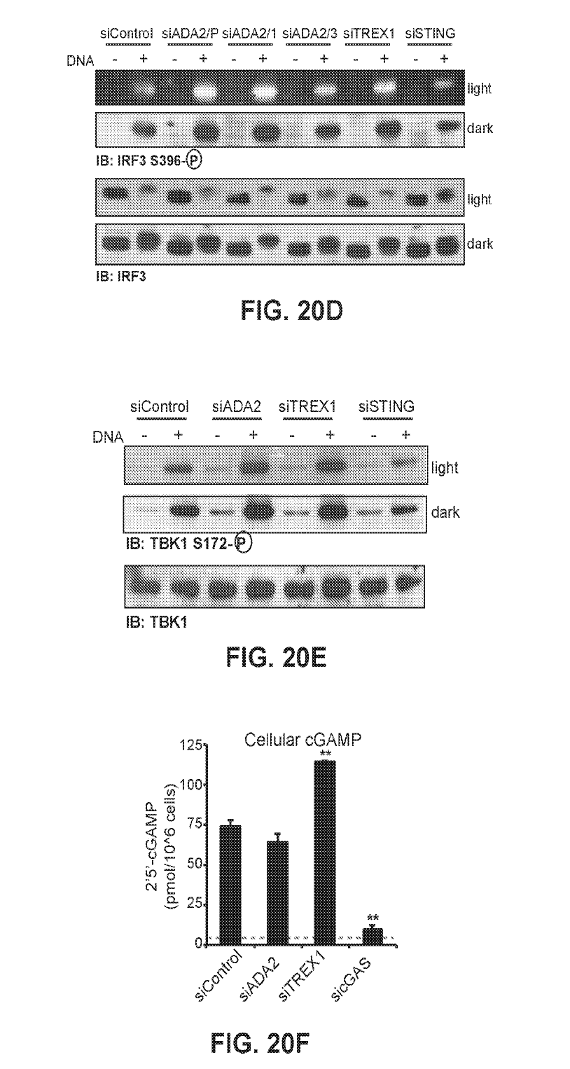

To examine specific profiles of DNA methylation in ADA2-depleted cells, the inventors performed whole genome sequencing after bisulfite treatment ˜73% of gene alterations observed in ADA2-depleted cells (FIG. 17h) were also accounted for in TREX1-depleted cells (FIG. 20a). Accordingly, STING mRNA levels were specifically up-regulated in cells depleted of siADA2 or supplemented with deoxynucleosides (FIG. 20a-b).

Several key signaling effectors of the IRF3 signaling pathway were over-expressed in ADA2-depleted cells, including MyD88, DDX58 (RIG-I), TMEM173 (STING) and MB21D1 (cGAS), and showed differential methylation within the promoter region or gene body (FIG. 20c). STING over-expression was unique to ADA2-depleted cells (FIG. 20b), and was confirmed to be up-regulated following dAdo and dIno supplementation in both resting and stimulated cells (FIG. 20d), suggesting that STING over-expression may drive enhanced IRF3 activation in ADA2-deficient cells. At the protein level, a ˜2-fold increase in STING was observed in ADA2-depleted cells, and correlated with increased TBK1 activation and IRF3 phosphorylation (FIGS. 20d-e). In contrast to cells lacking TREX1, which passively accumulate endogenous retroviral dsDNA elements in the cytoplasm resulting in activation of the cytoplasmic dsDNA sensor/enzyme cGAS, levels of 2′5′-cGAMP, the STING agonist produced by cGAS, were unchanged in siADA2-treated cells (FIG. 20f), emphasizing the distinct mechanism of action of ADA2 in regulating the cell-intrinsic innate immune response independently of spontaneous cytoplasmic dsDNA accumulation.

### Example 12

### Methods, Materials and Definitions

Antibodies and reagents: ADA2 antibody (immunocytochemistry, immunohistochemistry) was purchased from Cloud Clone Corp; ADA2 antibody (western blotting) from Sigma; β-actin, phospho-IRF3S396, phospho-TBK1S172, IRF3, TBK1, and STING antibodies were purchased from Santa Cruz. 2′ deoxyadenosine, adenosine and dipyridamole were purchased from Sigma; NECA from Tocris; Pentostatin from Cayman Chemicals. ELISA kit for ADA2 quantification was purchased from Cloud Clone Corp, 5-mC from ZyMed. Poly dA:dT and VACV70 dsDNA were synthesized by Integrated DNA Technologies (IDT).

Cells: Primary human cells were obtained from Lonza as single donor aliquots. Wherever possible, both male and female donors were analyzed. Human umbilical vascular endothelial cells (HUVEC) and Human aortic endothelial cells (HAEC) were maintained in EGM-2 Bulletkit growth media (Lonza) supplemented with 2% HI-FBS. Human dermal microvascular endothelial cells (HDMVEC), human brain microvascular endothelial cells (HBMVEC), and human kidney microvascular endothelial cells (HKMVEC) were maintained in EBM-2MV Bulletkit growth media (Lonza) supplemented with 5% HI-FBS. Normal human bronchial endothelial cells (NHBE) were maintained in BEGM Bulletkit growth media (Lonza). U937 were maintained in RPMI supplemented with 5% HI-FBS. For activation of cell-intrinsic innate immune responses through the STING-IFN-I pathway, all cells were stimulated for 3 h with poly dA:dT or VACV70 dsDNA (200-1000 ng/mL) using LyoVec as a transfection reagent (Invivogen), or infected for 3 h with hCMV MOLD at MOI=1.

Virus: The hCMV MOLD clinical strain was used. MOLD stocks were prepared as previously described⁷.

siRNA transfection: siRNA transfections were performed as previously described⁹. All siGenome and siOTP siRNAs were purchased from Dharmacon. Media was changed after 24 h and cells were harvested or analyzed after 72 h.

IRF3 immunostaining: Cells were fixed with 4% PFA for 20 min, washed 3 times with 1×PBS, permeabilized with 0.2% Triton X-100 for 10 min, and blocked with 10% FBS for 30 min Cells were incubated with IRF-3 primary antibody (clone FL-425, Santa Cruz) at 1:200 dilution for 30 min and washed 3 times with 1×PBS. Cells were then incubated with goat anti-rabbit Alexa-Fluor 647 secondary antibody (Jackson Immunoresearch) at 1:2000 dilution for 30 min, and washed 3 times with 1×PBS. DAPI was used to counter stain nuclei. Images were acquired at 10× magnification on ImageXpress Micro (Molecular Devices), and images were analyzed using the enhanced translocation module of MetaXpress (Molecular Devices). Individual cells were scored as positive for nuclear translocation if >70% of IRF3 fluorescence was spatially correlated with DAPI fluorescence. Each experimental data point is representative of >200 cells.

Real-time PCR: Total RNA was extracted using the Quick-RNA MiniPrep Plus kit (Zymo Research). Total RNA (200-1000 ng) was used to synthesize complementary DNA using the qScript cDNA synthesis kit (Quanta). Samples were diluted 10-fold and gene expression was analyzed by real-time quantitative PCR on a CFX96 Touch Detection System (Bio-Rad) using FastStart SYBR Green Master Mix (Roche) or TaqMan Universal PCR Master Mix (ThermoFisher Scientific).

Detection of IFNβ: For MSD assay, 25 ul of diluent and 25 ul of sample was dispensed in each well of MSD plate and incubated for 2 h with shaking at room temperature. The plate was then washed three times with 1× wash buffer. After wash, 25 ul of 1× detection antibody was added to each well and incubated for 2 h with shaking at room temperature. The plate was washed three times with 1× wash buffer. 150 ul of 2× read buffer t was added to each well and the plate was read on a MSD instrument. For IFN bioassay, HEK-blue IFN αHβ reporter cells (Invivogen) were cultured in DMEM media supplemented with 30 ug/ml blasticidin and 100 ug/ml of zeocine. 20 ul of sample was added to each well of a 96-well plate with 50,000 HEK-blue cells suspended in 180 ul of media and incubated for 24 h at 37° C. 20 ul of the induced HEK-blue IFN a/b cell supernatant was then incubated with 180 ul of Quanti blue and incubated for 1-3 h at 37° C. The plate was read in a spectrophotometer at 620-655 nm.

Lentivirus production for shRNA and CRISPR: For shRNA experiments, sequences were selected from the Sigma mission 2.0 library. For CRISPR experiments, sgRNA sequences selected using the CHOPCHOP algorithm and were cloned into the lentiCRISPrv2 vector (ref). Plasmids were transformed and amplified in Dh5α competent E. Coli, and purified using the EndoFree Plasmid Maxi kit (QIAGEN). For lentivirus preparation, 293T cells seeded in 6-well plates and transfected with pLKO.1 or lentiCRIPRv2 plasmids and MISSION Lentiviral Packaging Mix (Sigma), using JetPrime transfection reagent (Polyplus). After 24 h, media was changed to full growth media supplemented with 30% FBS. After 48 h, lentiviral supernatants were collected and HUVEC or U937 cells were spin-infected by centrifugation for 1 h at 37 C at 2000 rpm. Cells were selected in 3 ug/mL puromycin media for 2 weeks.

Flow cytometry: Cells were trypsinized and washed with FACS buffer (2% FBS in PBS) and stained with surface marker antibodies for 20 min on ice. Cells were washed twice with FACS buffer and data acquired on FACSCalibur (BD Biosciences). Analysis was done using FloJo software (Treestar).

RNAseq: Total RNA was extracted from 10\hat{ }7 cultured cells using the Quick-RNA MiniPrep Plus kit (Zymo Research). RNA quality was assessed using an Agilent Tapestation, such that all samples should produce RIN^{e} scores above 9.0. For each sample, 500-1000 ng of total RNA was then prepared into an mRNA library using the Truseq Stranded mRNA Library Prep Kit (Illumina). Resulting libraries were then pooled at equimolar concentrations using Quant-iT PicoGreen dsDNA Assay Kit (Life Technologies) and were deep sequenced on an Illumina 2500 in Rapid Run Mode, producing between 10M and 90M single-end reads with lengths of 50 nucleotides per sample.

The single-end reads that passed Illumina filters were filtered for reads aligning to tRNA, rRNA, adapter sequences, and spike-in controls. The reads were then aligned to UCSC hg19 reference genome using TopHat (v 1.4.1) [1]. DUST scores were calculated with PRINSEQ Lite (v 0.20.3) [2] and low-complexity reads (DUST>4) were removed from the BAM files. The alignment results were parsed via the SAMtools [3] to generate SAM files. Read counts to each genomic feature were obtained with the htseq-count program (v 0.6.0) [4] using the “union” option. After removing absent features (zero counts in all samples), the raw counts were then imported to R/Bioconductor package DESeq2 [5] to identify differentially expressed genes among samples. DESeq2 normalizes counts by dividing each column of the count table (samples) by the size factor of this column. The size factor is calculated by dividing the samples by geometric means of the genes. This brings the count values to a common scale suitable for comparison. P-values for differential expression are calculated using binomial test for differences between the base means of two conditions. These p-values are then adjusted for multiple test correction using Benjamin Hochberg algorithm [6] to control the false discovery rate. The inventors considered genes differentially expressed between two groups of samples when the DESeq2 analysis resulted in an adjusted P-value of <0.05 and the fold-change in gene expression was 2-fold. Cluster analyses including principal component analysis (PCA) and hierarchical clustering were performed using standard algorithms and metrics. Hierarchical clustering was performed using complete linkage with Euclidean metric.

Metabolite extraction and LC-MS/MS. For cell sample preparation, cells were lysed by aspirating in cold methanol, followed by three freeze-thaw cycles where samples were alternated between 40 C and −80 C baths in 30s intervals. Lysates are then centrifuged at 4 C at 14,000 rpm for 10 m to remove cellular debris with resulting supernatants dried using a Speed Vacuum system for two hours at 30 C before resuspension in an 80:20 ratio of methanol to water. For spent media samples, media was centrifuged at 14,000 rpm for one minute to remove cell debris before adding cold methanol at a ratio of 80:20 methanol to water. Cell and media samples were placed at −20 C for 30 m to precipitate protein before centrifugation at 14,000 RPM for 10 m at 4 C. Each injection for LC-MS/MS analysis was equivalent to 80,000 cells or 0.5 μL of media.

Liquid chromatography was performed with a Vanquish UHPLC system (Thermo Scientific), using a ZIC-pHILIC polymeric column (EMD Millipore) (150 mm×2.1 mm, 5 μm). Mobile phases used were (A) 20 mM ammonium bicarbonate in water, pH 9.6, and (B) acetonitrile. Mobile phase gradients were as follows: 90% B for first two 2 minutes followed by a linear gradient to 55% B at 16 minutes, sustained for an additional 3 minutes before final re-equilibration for 11 minutes at 90% B. Column temperature was 25 C, and mobile phase flow rate was 0.3 mL per minute.

Detection was performed using a QExactive Orbitrap mass spectrometer with heated electrospray ionization source (Thermo Scientific). Sheath and auxiliary gas was high purity nitrogen at 40 au (arbitrary units) and 20 au, respectively. Tandem mass spectra were collected in both positive and negative ionization polarities. Ion transfer tube was set at 275 C, and vaporizer temperature was set to 350 C. CID MS2 was collected using stepped normalized collision energies of 15, 30 and 45 arbitrary units and isolation widths of +/−0.5 mz. Peak heights from chromatograms corresponding to metabolites of interest were used for quantification. Indicated metabolites were detected as protonated/deprotonated ions of their monoisotopic masses and identities were confirmed by comparison to the MS1 and MS2 patterning of known standards under this LC-MS method.

### Methionine Labeling and SAM Flux:

Immunohistochemical staining of ADA2: Normal human formalin fixed paraffin embedded (FFPE) tissue sections were procured from USBiomax. During ADA2-CD31 double staining, modified method proposed by Kajimura et al (2016) was followed to reduce the problems associated with autofluoroscence present in 1-′1-PE tissue sections (1). At first, the FFPE tissues were deparaffinized by immersing them in Propar Clearant, 10 min×2 followed by sequential rehydration in 100%, 90%, 70% EtOH and PBST buffer for 5 m×2 each. Antigen retrieval was performed by boiling the tissues in TE Buffer (pH 9.0) for 30 m at 95° C. and gradually allowed to cool down in the same buffer for 45 minutes. Then to reduce autofluorescence the tissues were immersed in 0.25% NH₃/EtOH for 1 h, in 50% EtOH for 10 m and in rinsed with PBST 5 m×2 respectively. Then the slides were blocked with 10% goat serum for 1 hour at room temperature. Then again to reduce autofluorescence, slides were immersed in 3% H₂O₂ and washed with PBST for 5 min×2. Then anti CD31—anti ADA2 mixture (1:200) were added and kept in humidified chamber overnight at 4° C. Then after washing the tissue sections with PBST for 5 min×3, the tissues were treated with mixture of anti-mouse AF 488 (1:1000) and anti-rabbit AF 647 (1:1000) for 1 hour at room temperature. Then again, the tissues were washed with PBST thrice for 5 minutes. Further the tissues were immersed in 0.3% Sudan Black B for 1 hour followed by washing, 2 minute DAPI staining. Finally, the slides were mounted using Prolong Gold. Isotype, autofluoroscence, single stain and secondary only controls were also stained in the same procedure accordingly. Image acquisition for the stained tissues were performed using a multiphoton/confocal microscope (SP8 Leica®) at 40×/1.25 Oil objective. Maximum intensity projection images from Z stacks taken from CD31 and ADA2 double stained and controls were shown here. Some nonspecific binding of the secondary antibody to the tissues were seen but also specific signal for the ADA2 was also found above the background.

### Whole Genome Bisulfite Sequencing:

Statistics: Experiments were independently replicated at least three times. Data are reported as mean±standard deviation. Group or single measurement differences were determined by ANOVA (more than 3 groups) or Student's t-test (2 groups). p values equal or less than 0.05 were considered significant.

### Definitions

### AHCY—Adenosylhomocysteinase

### DNMT1—DNA Methyltransferase 1

### DNMT3a—DNA Methyltransferase 3 Alpha

### DNMT3b—DNA Methyltransferase 3 Beta

KF2345—an adenosine reuptake inhibitor—(3-[1-(6,7-diethoxy-2-morpholinoquinazolin-4-yl)piperidin-4-yl]-1,6-dimethyl-2,4(1H, 3H)-quinazolinedione hydrochloride) r75231—a nucleoside transport inhibitor (2-(aminocarbonyl)-N—(4-amino-2,6-dichlorophenyl (−4-[5,5-bis-(4-fluorophenyl)pentyl]-1-piperazineacetamide) STING—Stimulator of Interferon Genes protein, aka transmembrane protein 173

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation, and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”,“at least 97%”,“at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the technology herein has been specifically disclosed by representative embodiments and optional features, modification and variation of the concepts herein disclosed can be resorted to by those skilled in the art, and such modifications and variations are considered within the scope of this technology.

### REFERENCES

1 Navon Elkan, P. et al. Mutant adenosine deaminase 2 in a polyarteritis nodosa vasculopathy. The New England journal of medicine 370, 921-931, doi:10.1056/NEJMoa1307362 (2014).
2 Zhou, Q. et al. Early-onset stroke and vasculopathy associated with mutations in ADA2. The New England journal of medicine 370, 911-920, doi:10.1056/NEJMoa1307361 (2014).
3 Zavialov, A. V. & Engstrom, A. Human ADA2 belongs to a new family of growth factors with adenosine deaminase activity. The Biochemical journal 391, 51-57, doi:10.1042/BJ20050683 (2005).
4 Chen, Q., Sun, L. & Chen, Z. J. Regulation and function of the cGAS-STING pathway of cytosolic DNA sensing. Nature immunology 17, 1142-1149, doi:10.1038/ni.3558 (2016).
5 Stark, G. R. How cells respond to interferons revisited: from early history to current complexity. Cytokine & growth factor reviews 18, 419-423, doi:10.1016/j.cytogfr.2007.06.013 (2007).
6 Stark, G. R., Kerr, I. M., Williams, B. R., Silverman, R. H. & Schreiber, R. D. How cells respond to interferons. Annual review of biochemistry 67, 227-264, doi:10.1146/annurev.biochem.67.1.227 (1998).
7 Lio, C. W. et al. cGAS-STING Signaling Regulates Initial Innate Control of Cytomegalovirus Infection. J Virol 90, 7789-7797, doi:10.1128/JVI.01040-16 (2016).
8 Sharma, S. et al. An siRNA screen for NFAT activation identifies septins as coordinators of store-operated Ca2+ entry. Nature 499, 238-242, doi:10.1038/nature12229 (2013).
9 Thanos, D. & Maniatis, T. Virus induction of human IFN beta gene expression requires the assembly of an enhanceosome. Cell 83, 1091-1100 (1995).
10 Crow, Y. J. & Manel, N. Aicardi-Goutières syndrome and the type I interferonopathies. Nature reviews. Immunology 15, 429-440, doi:10.1038/nri3850 (2015).
11 Stoffels, M. & Kastner, D. L. Old Dogs, New Tricks: Monogenic Autoinflammatory Disease Unleashed. Annual review of genomics and human genetics 17, 245-272, doi:10.1146/annurev-genom-090413-025334 (2016).
12 Crow, Y. J. & Casanova, J. L. STING-associated vasculopathy with onset in infancy—a new interferonopathy. The New England journal of medicine 371, 568-571, doi:10.1056/NEJMe1407246 (2014).
13 Liu, Y. et al. Activated STING in a vascular and pulmonary syndrome. The New England journal of medicine 371, 507-518, doi:10.1056/NEJMoa1312625 (2014).
14 Crow, Y. J. et al. Mutations in the gene encoding the 3′-5′ DNA exonuclease TREX1 cause Aicardi-Goutières syndrome at the AGS1 locus. Nature genetics 38, 917-920, doi:10.1038/ng1845 (2006).
15 Lehtinen, D. A., Harvey, S., Mulcahy, M. J., Hollis, T. & Perrino, F. W. The TREX1 double-stranded DNA degradation activity is defective in dominant mutations associated with autoimmune disease. The Journal of biological chemistry 283, 31649-31656, doi:10.1074/jbc.M806155200 (2008).
16 Stetson, D. B., Ko, J. S., Heidmann, T & Medzhitov, R. Trex1 prevents cell-intrinsic initiation of autoimmunity. Cell 134, 587-598, doi:10.1016/j.cell.2008.06.032 (2008).
17 Pagnoux, C., Cohen, P. & Guillevin, L. Vasculitides secondary to infections. Clinical and experimental rheumatology 24, S71-81 (2006).
18 Kouchi, M. et al. A case of polyarteritis nodosa associated with cytomegalovirus infection. Case reports in rheumatology 2014, 604874, doi:10.1155/2014/604874 (2014).
19 Belot, A. et al. Mutations in CECR1 associated with a neutrophil signature in peripheral blood. Pediatric rheumatology online journal 12, 44, doi:10.1186/1546-0096-12-44 (2014).
20 Ramakers, B. P. et al. How systemic inflammation modulates adenosine metabolism and adenosine receptor expression in humans in vivo. Critical care medicine 40, 2609-2616, doi:10.1097/CCM.0b013e318259205b (2012).
21 Linden, J. Regulation of leukocyte function by adenosine receptors. Advances in pharmacology 61, 95-114, doi:10.1016/B978-0-12-385526-8.00004-7 (2011).
22 Smolenski, R. T., Kochan, Z., McDouall, R., Seymour, A. M. & Yacoub, M. H. Adenosine uptake and metabolism in human endothelial cells. Advances in experimental medicine and biology 370, 435-438 (1994).
23 Pearson, J. D., Carleton, J. S., Hutchings, A. & Gordon, J. L. Uptake and metabolism of adenosine by pig aortic endothelial and smooth-muscle cells in culture. The Biochemical journal 170, 265-271 (1978).
24 Kolassa, N., Pfleger, K. & Tram, M. Species differences in action and elimination of adenosine after dipyridamole and hexobendine. European journal of pharmacology 13, 320-325 (1971).
25 Jain, M. et al. Metabolite profiling identifies a key role for glycine in rapid cancer cell proliferation. Science 336, 1040-1044, doi:10.1126/science.1218595 (2012).
26 Szabados, E., Duggleby, R. G. & Christopherson, R. I. Metabolism of adenosine and deoxyadenosine by human erythrocytes and CCRF-CEM leukemia cells. The international journal of biochemistry & cell biology 28, 1405-1415 (1996).
27 Adrian, G. S. & Hutton, J. J. Adenosine deaminase messenger RNAs in lymphoblast cell lines derived from leukemic patients and patients with hereditary adenosine deaminase deficiency. The Journal of clinical investigation 71, 1649-1660 (1983).
28 Chiappinelli, K. B. et al. Inhibiting DNA Methylation Causes an Interferon Response in Cancer via dsRNA Including Endogenous Retroviruses. Cell 164, 1073, doi:10.1016/j.cell.2015.10.020 (2016).

What is claimed is:

1. A method of modulating an immune response, the method comprising inhibiting adenosine reuptake, adenosine salvage or adenosine degradation pathways of a cell.

2. The method of claim 1, wherein the cell is an endothelial cell.

3. The method of claim 1 or claim 2, wherein the immune response is an innate immune response.

4. The method of any one of claims 1 to 3, wherein the method comprises modulating Adenosine Deaminase 2 expression or activity or Adenosine Deaminase 1 expression or activity.

5. The method of claim 4, wherein the method comprises inhibiting Adenosine Deaminase 1 expression or activity.

6. The method of claim 5, wherein the method comprises contacting the cell with an agent that inhibits Adenosine Deaminase 1 expression or activity.

7. The method of any one of claims 1 to 3, wherein the method comprises stimulating expression or activity of one or more of AHCY, DNMT1, DNMT3a and DNMT3b.

8. The method of claim 7, wherein the method comprises contacting the cell with an agent that stimulates expression or activity of one or more of AHCY, DNMT1, DNMT3a and DNMT3b.

9. The method of any one of claims 1 to 3, wherein the method comprises contacting the cell with an adenosine reuptake inhibitor.

10. The method of claim 9, wherein the adenosine reuptake inhibitor is an acadesine, acetate, barbiturates, benzodiazepines, calcium channel blockers, carbamazepine, carisoprodol, cilostazol, cyclobenzaprine, dilazep, dipyridamole, estradiol, ethanol, flumazenil, hexobendine, hydroxyzine, indomethacin, inosine, KF24345, meprobamate, nitrobenzylthioguanosine, nitrobenzylthioinosine, papaverine, pentoxifylline, phenothiazines, phenytoin, progesterone, propentofylline, propofol, puromycin, r75231, soluflazine, toyocamycin, tracazolate, or tricyclic antidepressant.

11. The method of claim 9, wherein the adenosine reuptake inhibitor is dipyridamole.

12. The method of any one of claims 1 to 11, wherein the method comprises inhibiting, blocking or decreasing an immune response.

13. The method of any one of claims 1 to 12, wherein the method comprises modulating an immune response in a subject having an autoimmune disorder or proinflammatory condition.

14. The method of claim 13, wherein the autoimmune disorder or pro-inflammatory condition is selected from the group consisting of polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-esophageal reflux disease, erythroderma, Behcet's disease, amyotrophic lateral sclerosis, transplantation, rheumatoid arthritis, juvenile rheumatoid arthritis, malignant rheumatoid arthritis, Drug-Resistant Rheumatoid Arthritis, Neuromyelitis optica, Kawasaki disease, polyarticular or systemic juvenile idiopathic arthritis, psoriasis, chronic obstructive pulmonary disease (COPD), Castleman's disease, asthma, allergic asthma, allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenic purpura, pernicious anemia, sensorineural hearing loss, idiopathic bilateral progressive sensorineural hearing loss, autoimmune polyglandular syndrome type I or type II, immune infertility and immune-mediated infertility.

15. The method of claim 13, wherein the autoimmune disorder or pro-inflammatory condition is a vasculitis syndrome.

16. The method of claim 15, wherein the vasculitis syndrome is polyarteritis nodosa (PAN).

17. The method of claim 15, wherein the vasculitis syndrome comprises an adenosine deaminase 2 deficiency (ADA2).

18. The method of claim 15, wherein the vasculitis syndrome comprises Aicardi-Goutières Syndrome (AGS), TREX1 (AGS1 vasculitis), or STING (SAVI vasculitis).

19. The method of claim 13, wherein the autoimmune disorder or pro-inflammatory condition is a symptom or side-effect of cancer.

20. The method of any one of claims 13 to 18, wherein the pro-inflammatory condition comprises an adenosine deaminase 2 deficiency.

21. The method of any one of claims 13 to 20, wherein the subject has, or is suspected of having a viral, bacterial or fungal infection.

22. The method of claim 21, wherein the viral infection comprises an infection of Hepatitis B virus (HBV), Hepatitis C virus (HCV), Epstein-Barr virus (EBV) or cytomegalovirus (hCMV).

23. The method of any one of claims 13 to 22, wherein the autoimmune disorder or pro-inflammatory condition is not asthma.

24. The method of any one of claims 6, 8 and 9, wherein the inhibitor is a protein, peptide, small molecules, antibody, bispecific antibody, antibody derivative, ligand mimetic, nucleic acid or pharmaceutical composition.

25. A method of treating a subject having or suspected of having an autoimmune disorder, or pro-inflammatory condition comprising:
a) providing a subject having, or suspected of having, an autoimmune disorder, or pro-inflammatory condition; and
b) administering a therapeutically effective amount of an adenosine reuptake inhibitor, adenosine salvage pathway inhibitor or adenosine degradation pathway inhibitor to the subject.

26. The method of claim 25, wherein the adenosine reuptake inhibitor is selected from the group consisting of acadesine, acetate, barbiturates, benzodiazepines, calcium channel blockers, carbamazepine, carisoprodol, cilostazol, cyclobenzaprine, dilazep, dipyridamole, estradiol, ethanol, flumazenil, hexobendine, hydroxyzine, indomethacin, inosine, KF24345, meprobamate, nitrobenzylthioguanosine, nitrobenzylthioinosine, papaverine, pentoxifylline, phenothiazines, phenytoin, progesterone, propentofylline, propofol, puromycin, r75231, soluflazine, toyocamycin, tracazolate, and tricyclic antidepressants.

27. The method of claim 25, wherein the adenosine reuptake inhibitor is dipyridamole.

28. The method of claim 25, wherein the method comprises administering an inhibitor of Adenosine Deaminase 1.

29. The method of claim 25, wherein the method comprises administering a stimulator of AHCY, DNMT1, DNMT3a and DNMT3b.

30. The method of claim 25, wherein the adenosine reuptake inhibitor, the adenosine, the deoxyadenosine, or the variant or derivative thereof, is administered by intravenous (i.v.) administration to the subject.

31. The method of any one of claim 25, wherein the adenosine reuptake inhibitor, the adenosine, the deoxyadenosine, or the variant or derivative thereof, is administered at a dose of 0.01 mg to 50 mg, 0.01 mg to 12 mg, 0.01 to 6 mg, 0.01 mg to 5 mg or 0.01 mg to 1 mg.

32. The method of any one of claims 25 to 31, wherein the autoimmune disorder or pro-inflammatory condition is selected from the group consisting of polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-esophageal reflux disease, erythroderma, Behcet's disease, amyotrophic lateral sclerosis, transplantation, Neuromyelitis Optica, rheumatoid arthritis, juvenile rheumatoid arthritis, malignant rheumatoid arthritis, Drug-Resistant Rheumatoid Arthritis, Kawasaki disease, polyarticular or systemic juvenile idiopathic arthritis, psoriasis, chronic obstructive pulmonary disease (COPD), Castleman's disease, asthma, allergic asthma, allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenic purpura, pernicious anemia, sensorineural hearing loss, idiopathic bilateral progressive sensorineural hearing loss, autoimmune polyglandular syndrome type I or type II, immune infertility and immune-mediated infertility.

33. The method of any one of claims 25 to 31, wherein the autoimmune disorder or pro-inflammatory condition is a vasculitis syndrome.

34. The method of claim 33, wherein the vasculitis syndrome is polyarteritis nodosa (PAN).

35. The method of claim 33, wherein the vasculitis syndrome comprises an adenosine deaminase 2 deficiency (ADA2).

36. The method of claim 33, wherein the vasculitis syndrome comprises Aicardi-Goutières Syndrome (AGS), TREX1 (AGS1 vasculitis), or STING (SAVI vasculitis).

37. The method of any one of claims 25 to 36, wherein the pro-inflammatory condition comprises an adenosine deaminase 2 deficiency.

38. The method of any one of claims 25 to 31, wherein the autoimmune disorder or pro-inflammatory condition is a symptom or side-effect of cancer.

39. The method of any one of claims 25 to 38, wherein the subject has, or is suspected of having a viral, bacterial or fungal infection.

40. The method of claim 39, wherein the viral infection comprises an infection of Hepatitis B virus (HBV), Hepatitis C virus (HCV), Epstein-Barr virus (EBV) or cytomegalovirus (hCMV).

41. The method of any one of claims 25 to 40, wherein the autoimmune disorder or pro-inflammatory condition is not asthma.

42. A method of modulating an immune response, the method comprising stimulating the adenosine reuptake, adenosine salvage or adenosine degradation pathways of a cell.

43. The method of claim 42, wherein the cell is an endothelial cell.

44. The method of claim 42 or claim 43, wherein the immune response is an innate immune response.

45. The method of any one of claims 42 to 44, wherein the method comprises modulating Adenosine Deaminase 2 expression or activity or Adenosine Deaminase 1 expression or activity.

46. The method of claim 45, wherein the method comprises stimulating Adenosine Deaminase 1 expression or activity.

47. The method of claim 46, wherein the method comprises contacting the cell with an agent that stimulates Adenosine Deaminase 1 expression or activity.

48. The method of any one of claims 42 to 44, wherein the method comprises inhibiting expression or activity of one or more of AHCY, DNMT1, DNMT3a and DNMT3b.

49. The method of claim 48, wherein the method comprises contacting the cell with an agent that inhibits expression or activity of one or more of AHCY, DNMT1, DNMT3a and DNMT3b.

50. The method of any one of claims 43 to 44, wherein the method comprises contacting the cell with an adenosine reuptake stimulator.

51. The method of any one of claims 42 to 50, wherein the method comprises increasing, stimulating, eliciting or promoting an immune response.

52. The method of any one of claims 42 to 51, wherein the method comprises modulating an immune response in a subject having a neoplasia, neoplastic disorder or cancer

53. The method of claim 52, wherein the neoplasia, neoplastic disorder or cancer comprises a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma.

54. The method of claim 52 or 53, wherein the neoplasia, neoplastic disorder or cancer comprises hematopoietic cells.

55. The method of claim 53, wherein the sarcoma comprises a lymphosarcoma, liposarcoma, osteosarcoma, chondrosarcoma, leiomyosarcoma, rhabdomyosarcoma or fibrosarcoma.

56. The method of any one of claims 52 to 55, wherein the neoplasia, neoplastic disorder or cancer comprises a myeloma, lymphoma or leukemia.

57. The method of any one of claims 52 to 56, wherein the neoplasia, neoplastic disorder or cancer comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer.

58. The method of claim 57, wherein the lung neoplasia, neoplastic disorder or cancer comprises small cell lung or non-small cell lung cancer.

59. The method of any one of claims 52 to 58, wherein the neoplasia, neoplastic disorder or cancer comprises a stem cell neoplasia, neoplastic disorder or cancer.

60. The method of any one of claims 52 to 59, wherein the method inhibits, or reduces relapse or progression of the neoplasia, neoplastic disorder or cancer.

61. A method of treating a subject having a neoplasia, neoplastic disorder or cancer comprising:
a) providing a subject having, or suspected of having, a neoplastic disorder; and
b) administering a therapeutically effective amount of a compound or composition that stimulates adenosine uptake or the adenosine salvage or degradation pathways.

62. A method of reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy, comprising administering to the subject an amount of a compound or composition that stimulates adenosine uptake or the adenosine salvage or degradation pathway sufficient to reduce or inhibit metastasis of the neoplasia, tumor, cancer or malignancy to other sites, or formation or establishment of metastatic neoplasia, tumor, cancer or malignancy at other sites distal from the primary neoplasia, tumor, cancer or malignancy.

63. The method of claim 61 or 62, wherein the neoplasia, neoplastic disorder, tumor, cancer or malignancy comprises a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma.

64. The method of any one of claims 61 to 63, wherein the neoplasia, neoplastic disorder, tumor, cancer or malignancy comprises hematopoietic cells.

65. The method of claim 63, wherein the sarcoma comprises a lymphosarcoma, liposarcoma, osteosarcoma, chondrosarcoma, leiomyosarcoma, rhabdomyosarcoma or fibrosarcoma.

66. The method of any one of claims 61 to 65, wherein the neoplasia, neoplastic disorder, tumor, cancer or malignancy comprises a myeloma, lymphoma or leukemia.

67. The method of any one of claims 61 to 66, wherein the neoplasia, neoplastic disorder, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer.

68. The method of claim 67, wherein the lung neoplasia, neoplastic disorder, tumor, cancer or malignancy comprises small cell lung or non-small cell lung cancer.

69. The method of any one of claims 61 to 68, wherein the neoplasia, neoplastic disorder, tumor, cancer or malignancy comprises a stem cell neoplasia, tumor, cancer or malignancy.

70. The method of any one of claims 61 to 69, wherein the method inhibits, or reduces relapse or progression of the neoplasia, neoplastic disorder, tumor, cancer or malignancy.

71. The method of any one of claims 61 to 70, further comprising administering an anti-cell proliferative, anti-neoplastic, anti-tumor, anti-cancer or immune-enhancing treatment or therapy.

72. The method of any one of claims 61 to 71, wherein the treatment results in partial or complete destruction of the neoplastic, tumor, cancer or malignant cell mass; a reduction in volume, size or numbers of cells of the neoplastic, tumor, cancer or malignant cell mass; stimulating, inducing or increasing neoplastic, tumor, cancer or malignant cell necrosis, lysis or apoptosis; reducing neoplasia, tumor, cancer or malignancy cell mass; inhibiting or preventing progression or an increase in neoplasia, tumor, cancer or malignancy volume, mass, size or cell numbers; or prolonging lifespan.

73. The method of any one of claims 61 to 72, wherein the treatment results in reducing or decreasing severity, duration or frequency of an adverse symptom or complication associated with or caused by the neoplasia, tumor, cancer or malignancy.

74. The method of any one of claims 61 to 73, wherein the treatment results in reducing or decreasing pain, discomfort, nausea, weakness or lethargy.

75. The method of any one of claims 61 to 74, wherein the treatment results in increased energy, appetite, improved mobility or psychological well-being.

76. The method of any one of claims 61 to 75, the method comprising modulating Adenosine Deaminase 2 expression or activity or Adenosine Deaminase 1 expression or activity.

77. The method of claim 76, wherein the method comprises stimulating Adenosine Deaminase 1 expression or activity.

78. The method of claim 77, wherein the method comprises contacting the cell with an agent that stimulates Adenosine Deaminase 1 expression or activity.

79. The method of any one of claims 61 to 78, wherein the method comprises inhibiting expression or activity of one or more of AHCY, DNMT1, DNMT3a and DNMT3b.

80. The method of claim 79, wherein the method comprises contacting the cell with an agent that inhibits expression or activity of one or more of AHCY, DNMT1, DNMT3a and DNMT3b.

81. The method of any one of claims 61 to 80, wherein the method comprises contacting the cell with an adenosine reuptake stimulator.

82. A method of treating a subject having or suspected of having an autoimmune disorder, or pro-inflammatory condition comprising:
a) providing a subject having, or suspected of having, an autoimmune disorder, or pro-inflammatory condition; and
b) stimulating expression or activity of one or more of AHCY, DNMT1, DNMT3a and DNMT3b in the subject.

83. The method of claim 82, wherein the method comprises administering to the subject a therapeutically effective amount of an agent that stimulates expression or activity of one or more of AHCY, DNMT1, DNMT3a and DNMT3b.

84. A method of treating a subject having a neoplasia, neoplastic disorder or cancer comprising:
a) providing a subject having, or suspected of having, a neoplastic disorder; and
b) administering a therapeutically effective amount of an inhibitor of expression or activity of one or more of AHCY, DNMT1, DNMT3a and DNMT3b.

85. The method of claim 84, wherein the method comprises administering to the subject a therapeutically effective amount of an agent that inhibits expression or activity of one or more of AHCY, DNMT1, DNMT3a and DNMT3b.

86. A method of treating a subject having or suspected of having an autoimmune disorder, or pro-inflammatory condition comprising:
a) providing a subject having, or suspected of having, an autoimmune disorder, or pro-inflammatory condition; and
b) administering a therapeutically effective amount of an analogue of adenosine, deoxyadenosine, a variant or derivative thereof, to the subject.

87. The method of claim 86, wherein the analogue inhibits the activity of adenosine, or deoxyadenosine.

88. A method of treating a subject having a neoplasia, neoplastic disorder or cancer comprising:
a) providing a subject having, or suspected of having a neoplasia, neoplastic disorder or cancer; and
b) administering a therapeutically effective amount of adenosine, deoxyadenosine, or a variant or derivative thereof, or an analogue of adenosine or deoxyadenosine to the subject.

89. The method of claim 88, wherein the analogue of adenosine or deoxy adenosine mimics or agonizes the biological effects of adenosine, or deoxyadenosine.
